(19) United States
(12) Patent Application Publication
(10) Pub. No.: US 2019/0046552 A1
(43) Pub. Date: Feb. 14, 2019

(54) COMPOSITIONS AND METHODS FOR THE TREATMENT OF HCV INFECTION

(71) Applicant: Spring Bank Pharmaceuticals, Inc., Milford, MA (US)

(72) Inventor: Radhakrishnan P. Iyer, Shrewsbury, MA (US)

(21) Appl. No.: 15/565,078

(22) PCT Filed: Apr. 7, 2016
(86) PCT No.: PCT/US16/26504

§ 371 Date: Oct. 6, 2017

Related U.S. Application Data
(60) Provisional application No. 62/215,543, filed on Sep. 8, 2015.
(60) Provisional application No. 62/215,618, filed on Sep. 8, 2015.
(60) Provisional application No. 62/169,931, filed on Jun. 2, 2015.
(60) Provisional application No. 62/144,299, filed on Apr. 7, 2015.

(51) Int. Cl. A61K 31/7076; A61K 31/7072; A61K 45/06; A61P 31/14

(52) U.S. Cl. CPC A61K 31/7076; A61K 31/7072; A61K 2300/00; A61P 31/14; A61K 45/06

(57) ABSTRACT

In one aspect, the present invention features a method of treating a subject infected with the Hepatitis C virus, the method comprising administering to the subject a pharmaceutical composition comprising a compound of Formula (I) at a dosage of about 10 mg to about 1500 mg, wherein the compound is selected from Formula Ia, Formula Ib, and Formula Ic, or a prodrug or pharmaceutically acceptable salt thereof to thereby treat the subject. In another aspect, the present invention features a kit comprising a pharmaceutical composition comprising the compound of Formula (I). In some embodiments, the kit further comprises an additional agent or treatment.

### RELATED APPLICATIONS

This applications claims priority to U.S. Provisional Application No. 62/215,618, filed on Sep. 8, 2015; U.S. Provisional Application No. 62/215,543, filed on Sep. 8, 2015; U.S. Provisional Application No. 62/169,931, filed on Jun. 2, 2015; and U.S. Provisional Application No. 62/144,299, filed on Apr. 7, 2015. The entire disclosures of each of the foregoing applications are incorporated herein by reference.

### FIELD OF INVENTION

This invention relates to methods useful in the treatment of HCV infection.

### BACKGROUND OF INVENTION

Hepatitis C virus (HCV) is a major cause of liver disease worldwide, with nearly 170 million people infected and about four million new infections each year (Shephard, C. W. et al, Lancet Infect Dis (2005) 5:558-567). About 80% of acutely infected HCV patients progress to chronic infection, 20% of whom develop cirrhosis within 25 years and have increased likelihood of liver failure and hepatocellular carcinoma (Kohli, A. et al, J Am Med Assoc (2014) 312:631-640). HCV is the leading cause of liver transplantation in the United States.

HCV is a single-stranded enveloped RNA virus most commonly transmitted by blood-to-blood contact (e.g., unsafe injection practices, inadequate sterilization of medical equipment, and exposure to unscreened blood and blood products). Current antiviral therapies result in severe toxicity and are effective in only a subset of patients, although treatment outcomes have recently been improved through combination therapy with drugs such as boceprevir and telaprevir, which target the HCV NS3/4A protease. Other antiviral drugs target the HCV polymerase, the HCV protease, and the HCV NS5A protein. Despite extensive efforts to treatments with increased potency and lowered toxicity, the long term efficacy of most HCV antiviral drugs is hampered by the rapid emergence of resistant mutants due to the high rate of error in the HCV replication cycle (Romano, K. P. et al PLOS Pathog (2012) 8: e1002832).

Further, a major obstacle for treatment of HCV infection relates to the emergence of drug resistant variants that occurs upon extended use of currently available nucleoside and nucleotide analogs. In addition, current treatments may require persistent and long-term use, which often results in unwarranted side effects and the risk of relapse upon treatment discontinuation. Accordingly, there is a critical need for a new generation of therapies to combat HCV infection.

### SUMMARY OF INVENTION

In one aspect, the present invention features a method of treating a subject infected with the Hepatitis C virus, the method comprising administering to the subject a pharmaceutical composition comprising a compound of Formula (I) at a dosage of about 10 mg to about 1500 mg, wherein the compound is selected from:

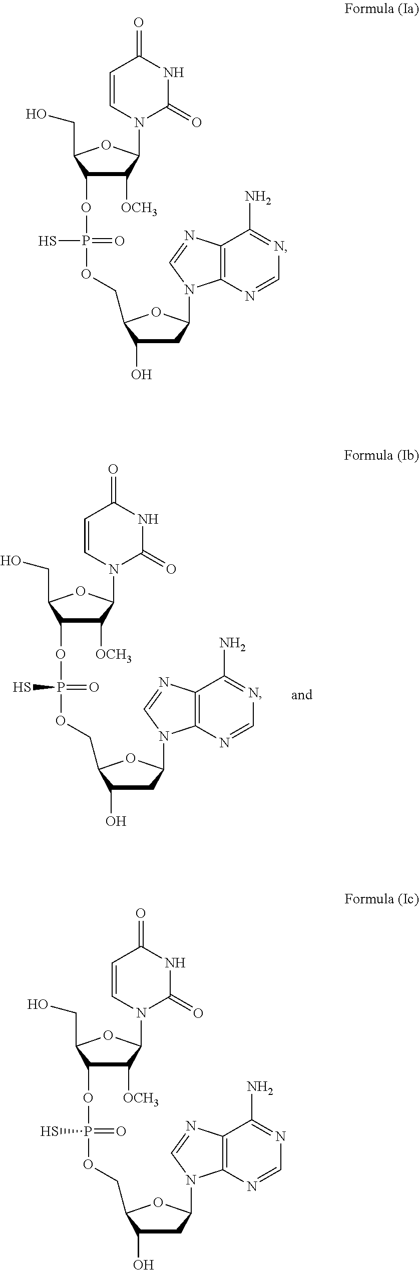

or a prodrug or pharmaceutically acceptable salt thereof to thereby treat the subject. In some embodiments, the prodrug of Formula (I) is a compound of Formula (II), wherein the compound is selected from:

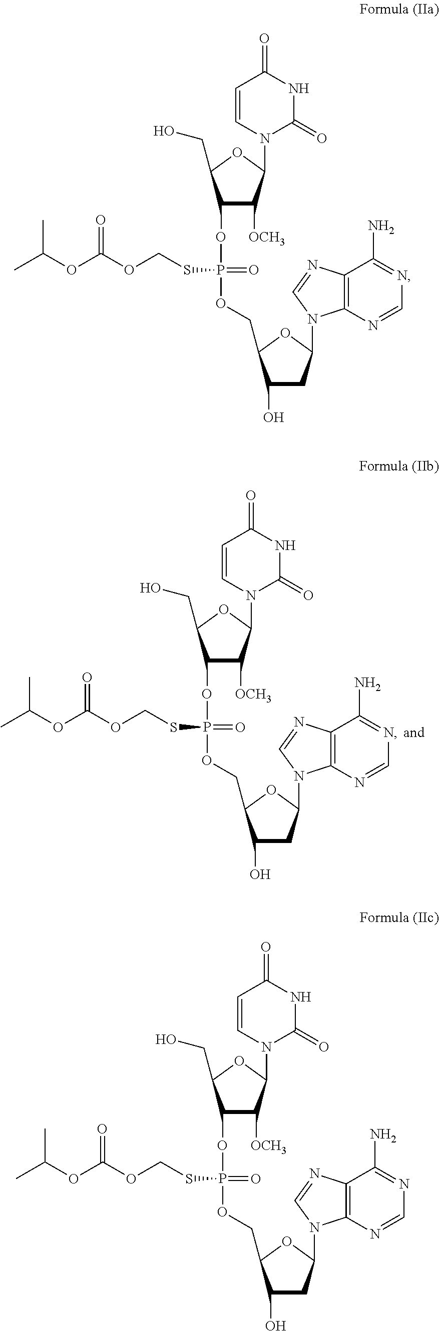

or a pharmaceutically acceptable salt thereof.

In some embodiments, the composition comprises a mixture of compounds of Formula (I). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the composition comprises a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the composition is administered orally. In some embodiments, the compound of Formula (I) or Formula (II) is administered orally. In some embodiments, the compound of Formula (II) is administered orally. In some embodiments, the composition is a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the composition is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of the composition is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of the composition is between about 50 mg and about 1000 mg. In some embodiments, the dosage of the composition is between about 200 mg and about 1000 mg.

In some embodiments, the composition is administered daily. In some embodiments, the composition is administered once daily. In some embodiments, the composition is administered more than once a day, e.g., twice a day, three times a day, four times a day. In some embodiments, the composition is administered every other day, every 2 days, every 3 days, every 4 days, or more.

In some embodiments, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, or HCV genotype 3.

In some embodiments, the subject is treatment naïve. In some embodiments, the subject has previously been treated for HCV infection.

In some embodiments, the subject is treated immediately after eating a meal. In some embodiments, the subject is treated about 5 minutes after eating a meal, about 10 minutes after, about 15 minutes after, about 30 minutes after, about 45 minutes after, about 1 hour after, about 1.5 hours after, about 2 hours after, about 3 hours after, about 4 hours after, about 6 hours after, about 8 hours after, about 12 hours after, about 16 hours after, about 1 day after eating a meal. In some embodiments, the subject is treated in the fed state. In some embodiments, the subject is treated after abstaining from food for about 30 minutes, about 45 minutes, about 1 hour, about 1.5 hours, 2 hours, about 3 hours, about 4 hours, about 6 hours, about 8 hours, about 12 hours, about 16 hours, about 1 day prior to treatment. In some embodiments, the subject is treated in the fasted state.

In another aspect, the present invention features a method of evaluating a subject, the method comprising administering to the subject a pharmaceutical composition comprising a compound of Formula (II), wherein the compound is selected from:

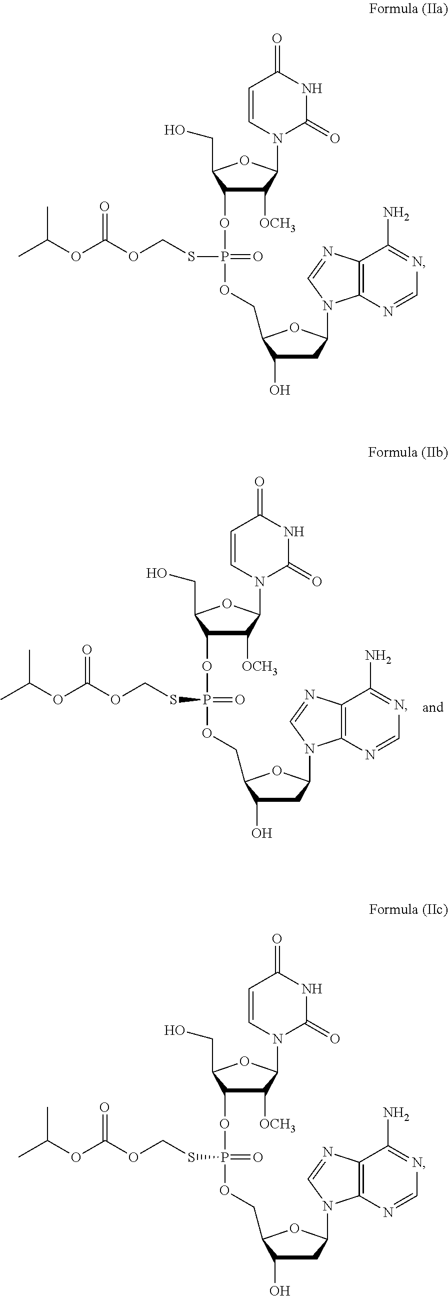

or a pharmaceutically acceptable salt thereof and acquiring the level of one or more of: a) a compound of Formula (II) or a salt thereof; b) a metabolite of a compound of Formula (II) (e.g., a compound of Formula (I)) or a salt thereof; and c) HCV RNA in the subject, to thereby evaluate the subject.

In some embodiments, the metabolite of a compound of Formula (II) is a compound of Formula (I), wherein the compound is selected from:

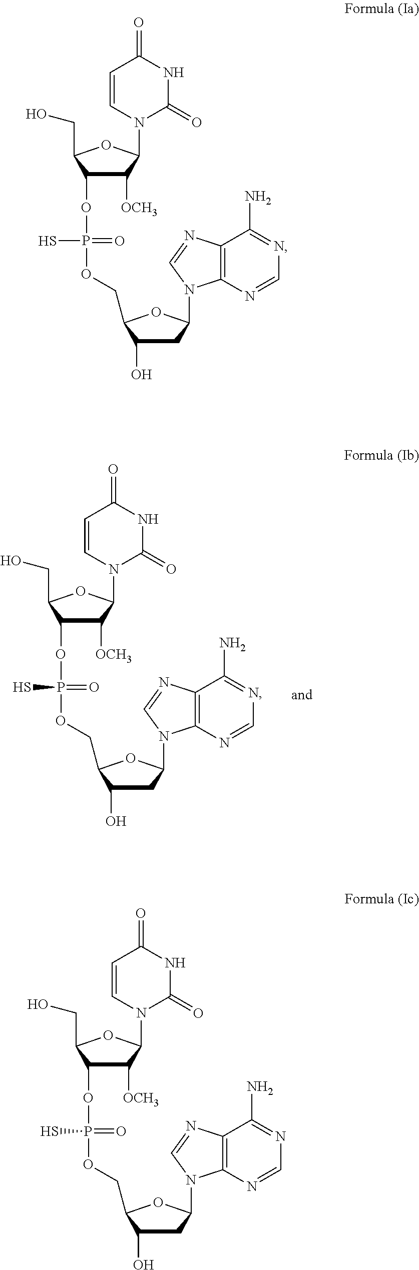

or a pharmaceutically acceptable salt thereof.

In some embodiments, the value of one or more of: a) a compound of Formula (II) or a salt thereof; b) a metabolite of a compound of Formula (II) (e.g., a compound of Formula (I)) or a salt thereof; and c) HCV RNA in the subject is acquired by analyzing a blood sample taken from a subject. In some embodiments, the value of one or more of: a) a compound of Formula (II) or a salt thereof; b) a metabolite of a compound of Formula (II) (e.g., a compound of Formula (I)) or a salt thereof; and c) HCV RNA in the subject is acquired by analyzing the plasma concentration of each in a blood sample taken from a subject. In some embodiments, the analysis is performed by sample analysis of a bodily fluid, such as blood, by mass spectrometry (e.g., LC-MS) or PCR (e.g., RT-PCR).

In some embodiments, one or more of: a) a compound of Formula (II) or a salt thereof; and b) a metabolite of a compound of Formula (II) (e.g., a compound of Formula (I)) or a salt thereof is detectable in the plasma of the subject within about 30 minutes to about 8 hours after administration of the composition. In some embodiments, one or more of a) a compound of Formula (II) or a salt thereof; and b) a metabolite of a compound of Formula (II) (e.g., a compound of Formula (I)) or a salt thereof is detectable in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition.

In some embodiments, one or more of: a) a compound of Formula (II) or a salt thereof; and b) a metabolite of a compound of Formula (II) (e.g., a compound of Formula (I)) or a salt thereof is detectable at a peak level in the plasma of the subject within about 30 minutes to about 8 hours after administration of the composition. In some embodiments, one or more of: a) a compound of Formula (II) or a salt thereof; and b) a metabolite of a compound of Formula (II) (e.g., a compound of Formula (I)) or a salt thereof is detectable at a peak level in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition. In some embodiments, the peak level of said compounds is detectable between 1 and 6 hours after administration of the composition.

In some embodiments, the composition comprises a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the composition is administered orally. In some embodiments, the compound Formula (II) is administered orally. In some embodiments, the composition is a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the composition is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of the composition is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of the composition is between about 50 mg and about 1000 mg. In some embodiments, the dosage of the composition is between about 200 mg and about 1000 mg.

In some embodiments, the composition is administered daily. In some embodiments, the composition is administered once daily. In some embodiments, the composition is administered more than once a day, e.g., twice a day, three times a day, four times a day. In some embodiments, the composition is administered every other day, every 2 days, every 3 days, every 4 days, or more.

In some embodiments, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, or HCV genotype 3.

In some embodiments, the subject is treatment naïve. In some embodiments, the subject has previously been treated for HCV infection.

In some embodiments, the subject is treated immediately after eating a meal. In some embodiments, the subject is treated about 5 minutes after eating a meal, about 10 minutes after, about 15 minutes after, about 30 minutes after, about 45 minutes after, about 1 hour after, about 1.5 hours after, about 2 hours after, about 3 hours after, about 4 hours after, about 6 hours after, about 8 hours after, about 12 hours after, about 16 hours after, about 1 day after eating a meal. In some embodiments, the subject is treated in the fed state. In some embodiments, the subject is treated after abstaining from food for about 30 minutes, about 45 minutes, about 1 hour, about 1.5 hours, 2 hours, about 3 hours, about 4 hours, about 6 hours, about 8 hours, about 12 hours, about 16 hours, about 1 day prior to treatment. In some embodiments, the subject is treated in the fasted state.

In another aspect, the present invention features a method of evaluating a subject, the method comprising acquiring (e.g., directly acquiring) the value for the level of one or more of: a) a compound of Formula (II) or salt thereof; b) a metabolite of a compound of Formula (II) (e.g., a compound of Formula (I)) or a salt thereof; and c) HCV RNA in a subject that has been administered a composition comprising a compound of Formula (II), wherein the compound is selected from:

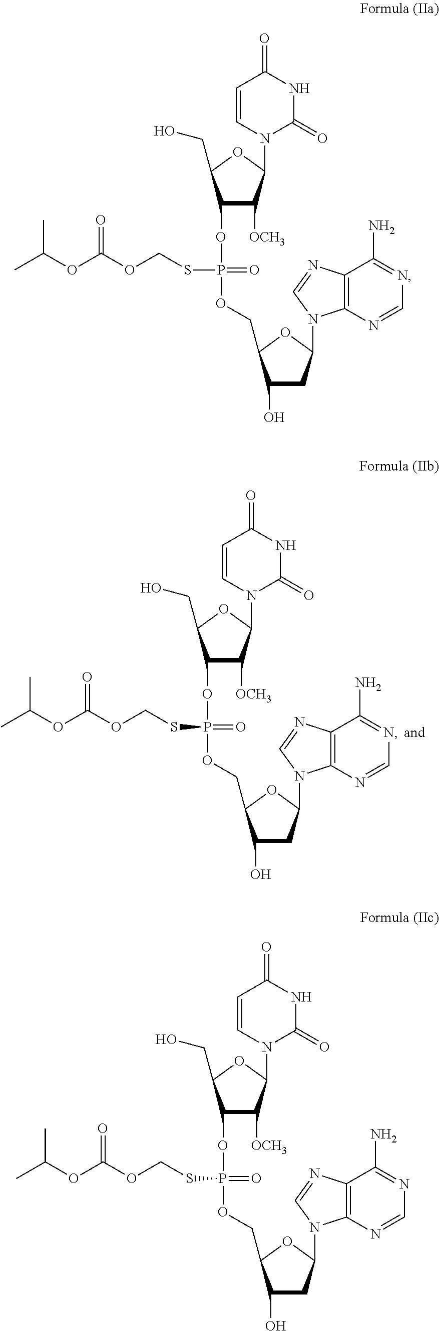

or a pharmaceutically acceptable salt thereof.

In some embodiments, the acquiring comprises receiving a sample directly from a subject. In some embodiments, the acquiring comprises transmitting the value to another party, e.g., the party that administered the composition.

In some embodiments, the metabolite of a compound of Formula (II) is a compound of Formula (I), wherein the compound is selected from:

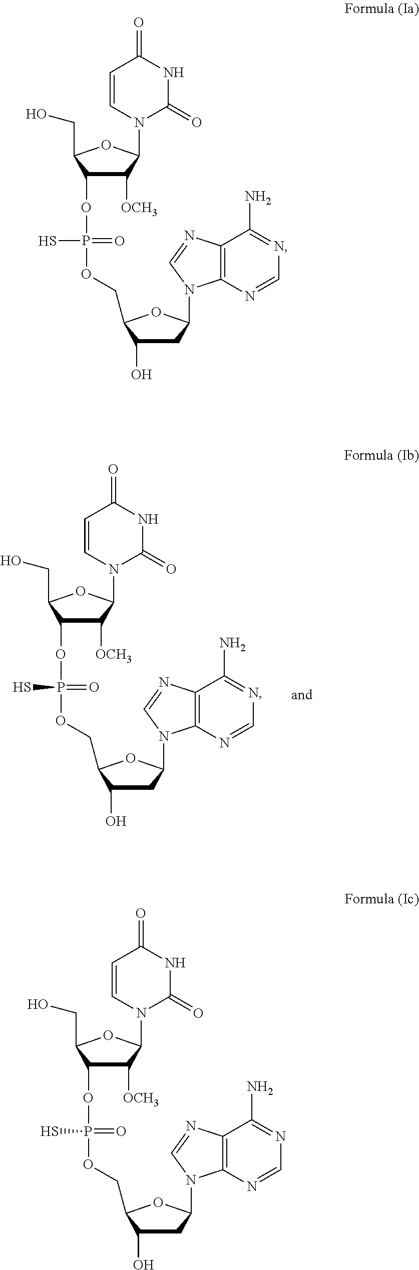

or a salt thereof.

In some embodiments, the value of one or more of: a) a compound of Formula (II) or a salt thereof; b) a metabolite of a compound of Formula (II) (e.g., a compound of Formula (I)) or a salt thereof; and c) HCV RNA in the subject is acquired by analyzing a blood sample taken from a subject. In some embodiments, the value of one or more of: a) a compound of Formula (II) or a salt thereof; b) a metabolite of a compound of Formula (II) (e.g., a compound of Formula (I)) or a salt thereof; and c) HCV RNA in the subject is acquired by analyzing the plasma concentration of each in a blood sample taken from a subject. In some embodiments, the analysis is performed by sample analysis of a bodily fluid, such as blood, by mass spectrometry (e.g., LC-MS) or PCR (e.g., RT-PCR).

In some embodiments, one or more of: a) a compound of Formula (II) or a salt thereof; and b) a metabolite of a compound of Formula (II) (e.g., a compound of Formula (I)) or a salt thereof is detectable in the plasma of the subject within about 30 minutes to about 8 hours after administration of the composition. In some embodiments, one or more of: a) a compound of Formula (II) or salt thereof; and b) a metabolite of a compound of Formula (II) (e.g., a compound of Formula (I)) or a salt thereof is detectable in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition.

In some embodiments, one or more of: a) a compound of Formula (II) or a salt thereof; and b) a metabolite of a compound of Formula (II) (e.g., a compound of Formula (I)) or a salt thereof is detectable at a peak level in the plasma of the subject within about 30 minutes to about 8 hours after administration of the composition. In some embodiments, one or more of: a) a compound of Formula (II) or a salt thereof; and b) a metabolite of a compound of Formula (II) (e.g., a compound of Formula (I)) or a salt thereof is detectable at a peak level in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition. In some embodiments, the peak level of said compounds is detectable between 1 and 6 hours after administration of the composition.

In some embodiments, the composition comprises a mixture of compounds of Formula (II). In some embodiments, the composition comprises a mixture of Formula (IIb) and Formula (IIc). In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (IIb) and comprises less than about 5% of Formula (IIc), e.g., less than about 4%, less than about 3%, less than about 2%, less than about 1%, less than about 0.5%, or less than about 0.1% of Formula (IIc), or is substantially free of Formula (IIc). In some embodiments, the composition comprises Formula (IIc) and comprises less than about 5% of Formula (IIb), e.g., less than about 4%, less than about 3%, less than about 2%, less than about 1%, less than about 0.5%, or less than about 0.1% of Formula (IIb), or is substantially free of Formula (IIb).

In some embodiments, the composition is administered orally. In some embodiments, the compound of Formula (II) is administered orally. In some embodiments, the composition is a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the composition is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of the composition is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of the composition is between about 50 mg and about 1000 mg. In some embodiments, the dosage of the composition is between about 200 mg and about 1000 mg.

In some embodiments, the composition is administered daily. In some embodiments, the composition is administered once daily. In some embodiments, the composition is administered more than once a day, e.g., twice a day, three times a day, four times a day. In some embodiments, the composition is administered every other day, every 2 days, every 3 days, every 4 days, or more.

In some embodiments, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, or HCV genotype 3.

In some embodiments, the subject is treatment naïve. In some embodiments, the subject has previously been treated for HCV infection.

In some embodiments, the subject is treated immediately after eating a meal. In some embodiments, the subject is treated about 5 minutes after eating a meal, about 10 minutes after, about 15 minutes after, about 30 minutes after, about 45 minutes after, about 1 hour after, about 1.5 hours after, about 2 hours after, about 3 hours after, about 4 hours after, about 6 hours after, about 8 hours after, about 12 hours after, about 16 hours after, about 1 day after eating a meal. In some embodiments, the subject is treated in the fed state. In some embodiments, the subject is treated after abstaining from food for about 30 minutes, about 45 minutes, about 1 hour, about 1.5 hours, 2 hours, about 3 hours, about 4 hours, about 6 hours, about 8 hours, about 12 hours, about 16 hours, about 1 day prior to treatment. In some embodiments, the subject is treated in the fasted state.

In another aspect, the present invention features a method of evaluating a subject, the method comprising administering to the subject a pharmaceutical composition comprising a compound of Formula (I), wherein the compound is selected from:

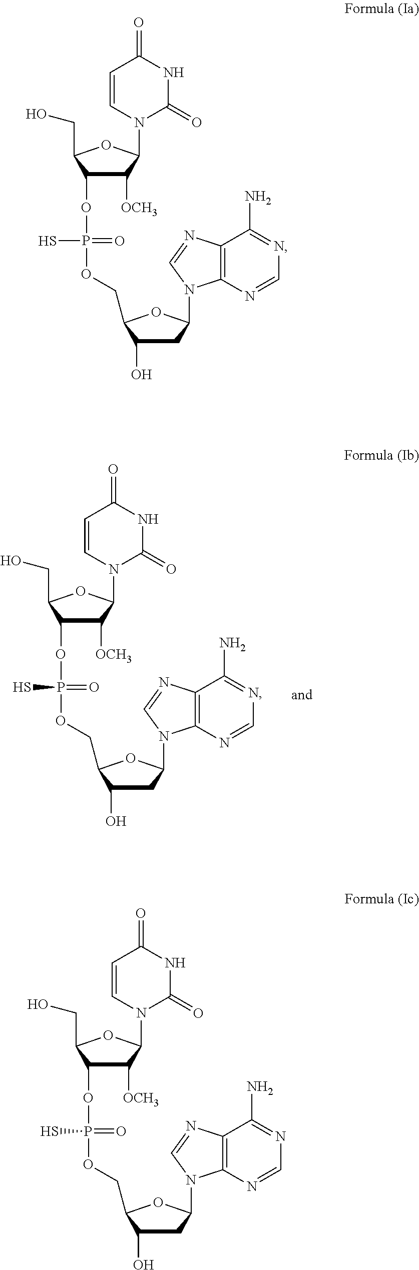

or prodrug or pharmaceutically acceptable salt thereof and acquiring the level of one or more of: a) a compound of Formula (I) or prodrug thereof; b) a metabolite of a compound of Formula (I) or a prodrug thereof; and c) HCV RNA in the subject, to thereby evaluate the subject.

In some embodiments, the value of one or more of: a) a compound of Formula (I) or a salt thereof; b) a metabolite of a compound of Formula (I) or a salt thereof; and c) HCV RNA in the subject is acquired by analyzing a blood sample taken from a subject. In some embodiments, the value of one or more of: a) a compound of Formula (I) or a salt thereof; b) a metabolite of a compound of Formula (I) or a salt thereof; and c) HCV RNA in the subject is acquired by analyzing the plasma concentration of each in a blood sample taken from a subject. In some embodiments, the analysis is performed by sample analysis of a bodily fluid, such as blood, by mass spectrometry (e.g., LC-MS) or PCR (e.g., RT-PCR).

In some embodiments, one or more of: a) a compound of Formula (I) or a salt thereof; and b) a metabolite of a compound of Formula (I) or a salt thereof is detectable in the plasma of the subject within about 30 minutes to about 8 hours after administration of the composition. In some embodiments, one or more of a) a compound of Formula (I) or a salt thereof; and b) a metabolite of a compound of Formula (I) or a salt thereof is detectable in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition.

In some embodiments, one or more of: a) a compound of Formula (I) or a salt thereof; and b) a metabolite of a compound of Formula (I) or a salt thereof is detectable at a peak level in the plasma of the subject within about 30 minutes to about 8 hours after administration of the composition. In some embodiments, one or more of: a) a compound of Formula (I) or a salt thereof; and b) a metabolite of a compound of Formula (I) or a salt thereof is detectable at a peak level in the plasma of the subject within about 30 minutes, within about 1 hour, within about 1.5 hours, within about 2 hours, within about 3 hours, within about 4 hours, within about 5 hours, within about 6 hours, within about 7 hours, or within about 8 hours after administration of the composition. In some embodiments, the peak level of said compounds is detectable between 1 and 6 hours after administration of the composition.

In another aspect, the present invention features a method of evaluating a subject, the method comprising acquiring (e.g., directly acquiring) the value for the level of one or more of: a) a compound of Formula (I) or prodrug thereof; b) a metabolite of a compound of Formula (I) or a prodrug thereof; and c) HCV RNA in a subject that has been administered a composition comprising a compound of Formula (I), wherein the compound is selected from:

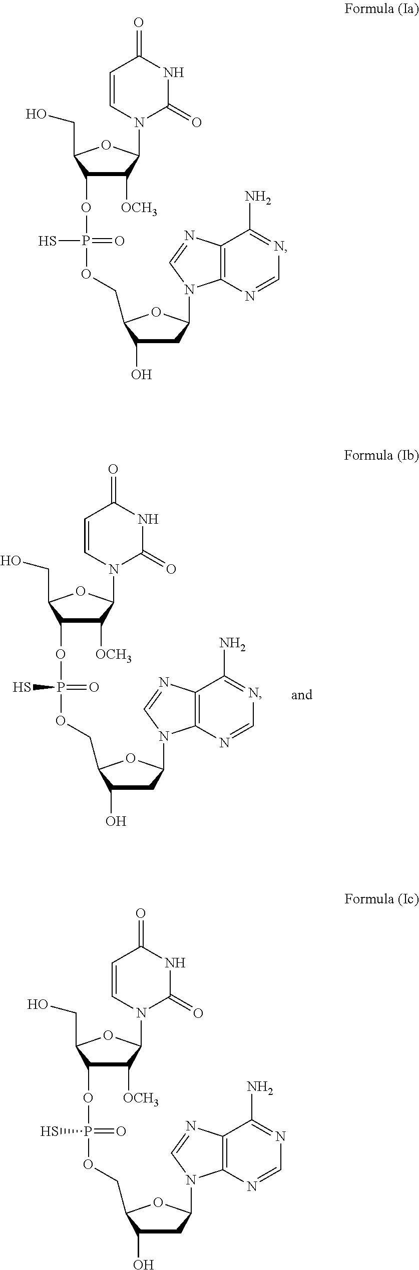

or a prodrug or pharmaceutically acceptable salt thereof.

In some embodiments, the acquiring comprises receiving a sample directly from a subject. In some embodiments, the acquiring comprises transmitting the value to another party, e.g., the party that administered the composition.

In some embodiments, the composition comprises a mixture of compounds of Formula (I). In some embodiments, the composition comprises a mixture of Formula (Ib) and Formula (Ic). In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the composition comprises Formula (Ib) and comprises less than about 5% of Formula (Ic), e.g., less than about 4%, less than about 3%, less than about 2%, less than about 1%, less than about 0.5%, or less than about 0.1% of Formula (Ic), or is substantially free of Formula (Ic). In some embodiments, the composition comprises Formula (Ic) and comprises less than about 5% of Formula (Ib), e.g., less than about 4%, less than about 3%, less than about 2%, less than about 1%, less than about 0.5%, or less than about 0.1% of Formula (Ib), or is substantially free of Formula (Ib).

In some embodiments, the composition is administered orally. In some embodiments, the compound of Formula (I) is administered orally. In some embodiments, the composition is a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, dragée, or powder.

In some embodiments, the dosage of the composition is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of the composition is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of the composition is between about 50 mg and about 1000 mg. In some embodiments, the dosage of the composition is between about 200 mg and about 1000 mg.

In some embodiments, the composition is administered daily. In some embodiments, the composition is administered once daily. In some embodiments, the composition is administered more than once a day, e.g., twice a day, three times a day, four times a day. In some embodiments, the composition is administered every other day, every 2 days, every 3 days, every 4 days, or more.

In some embodiments, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, or HCV genotype 3.

In some embodiments, the subject is treatment naïve. In some embodiments, the subject has previously been treated for HCV infection.

In some embodiments, the subject is treated immediately after eating a meal. In some embodiments, the subject is treated about 5 minutes after eating a meal, about 10 minutes after, about 15 minutes after, about 30 minutes after, about 45 minutes after, about 1 hour after, about 1.5 hours after, about 2 hours after, about 3 hours after, about 4 hours after, about 6 hours after, about 8 hours after, about 12 hours after, about 16 hours after, about 1 day after eating a meal. In some embodiments, the subject is treated in the fed state. In some embodiments, the subject is treated after abstaining from food for about 30 minutes, about 45 minutes, about 1 hour, about 1.5 hours, 2 hours, about 3 hours, about 4 hours, about 6 hours, about 8 hours, about 12 hours, about 16 hours, about 1 day prior to treatment. In some embodiments, the subject is treated in the fasted state.

In another aspect, the present invention features a method of evaluating a subject, the method comprising treating a subject infected with the Hepatitis C virus with a composition comprising a compound of Formula (I) or a prodrug or pharmaceutically acceptable salt thereof and acquiring information regarding the occurrence of an adverse event to thereby evaluate the subject.

In an embodiment, a method described herein is performed without the occurrence of a serious adverse event.

In another aspect, the present invention features a method of treating a subject infected with the Hepatitis C virus, the method comprising administering to the subject a dosage of a pharmaceutical composition sufficient to provide a blood concentration of HCV RNA reduced by at least about 5-fold relative to a reference standard, e.g., by at least about 6-fold, by at least about 7-fold, by at least about 8-fold, by at least about 9-fold, by at least about 10-fold, by at least about 15-fold, by at least about 20-fold, by at least about 30-fold, by at least about 50-fold, by at least about 75-fold, by at least about 100-fold, by at least about 500-fold, by at least about 1000-fold, by at least about 5000-fold or more.

In another aspect, the present invention features a method of treating a subject infected with the Hepatitis C virus, the method comprising administering to the subject a compound of Formula (I), wherein the compound is selected from:

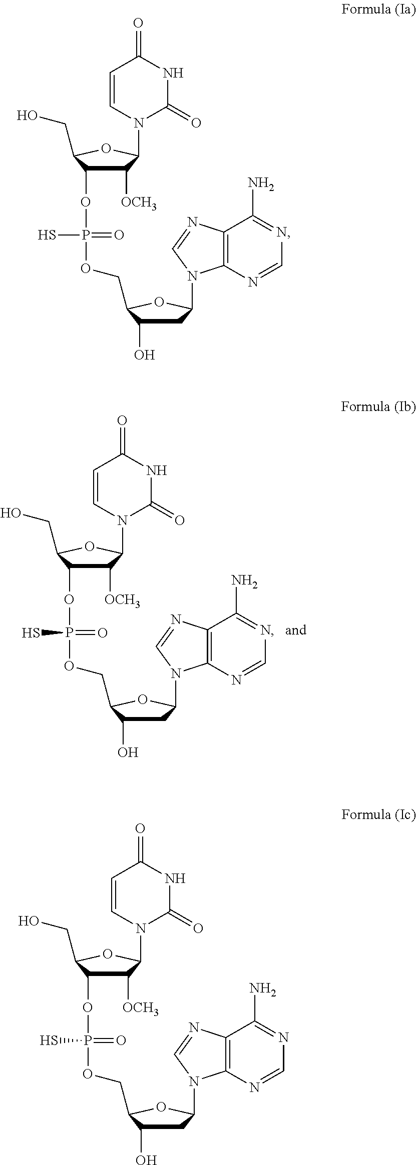

or a prodrug or pharmaceutically acceptable salt thereof in combination with sofosbuvir to thereby treat the subject. In some embodiments, the prodrug of Formula (I) is a compound of Formula (II), wherein the compound is selected from:

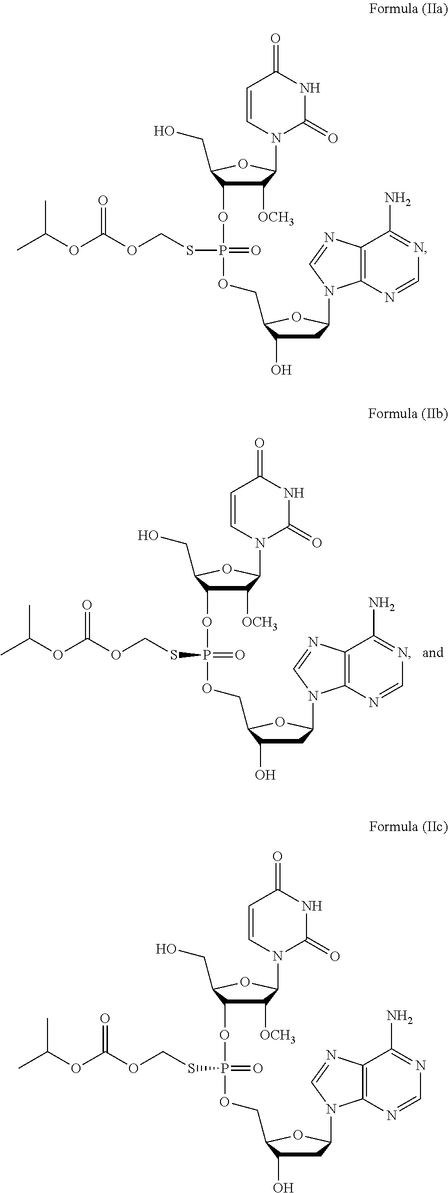

or a pharmaceutically acceptable salt thereof. In some embodiments, the method comprises administering to the subject a compound of Formula (I) or a pharmaceutically acceptable salt thereof in combination with sofosbuvir. In some embodiments, the method comprises administering to the subject a compound of Formula (II) or a pharmaceutically acceptable salt thereof in combination with sofosbuvir.

In some embodiments, the method described herein comprises administering to the subject a mixture of compounds of Formula (I) or pharmaceutically acceptable salts thereof in combination with sofosbuvir. In some embodiments, the method described herein comprises administering to the subject a mixture of compounds of Formula (Ib) and Formula (Ic) or pharmaceutically acceptable salts thereof in combination with sofosbuvir. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic), in combination with sofosbuvir. In some embodiments, the method described herein comprises administering to the subject a compound of Formula (I) comprising Formula (Ib) or a pharmaceutically acceptable salt thereof that is substantially free of Formula (Ic), in combination with sofosbuvir. In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib), in combination with sofosbuvir. In some embodiments, the method described herein comprises administering to the subject a compound of Formula (I) comprising Formula (Ic) or a pharmaceutically acceptable salt thereof that is substantially free of Formula (Ib), in combination with sofosbuvir.

In some embodiments, the method described herein comprises administering to the subject a mixture of compounds of Formula (II) or pharmaceutically acceptable salts thereof in combination with sofosbuvir. In some embodiments, the method described herein comprises administering to the subject a mixture of Formula (IIb) and Formula (IIc) or pharmaceutically acceptable salts thereof in combination with sofosbuvir. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc), in combination with sofosbuvir. In some embodiments, the method described herein comprises administering to the subject a compound of Formula (II) comprising Formula (IIb) or a pharmaceutically acceptable salt thereof that is substantially free of Formula (IIc), in combination with sofosbuvir. In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb), in combination with sofosbuvir. In some embodiments, the method described herein comprises administering to the subject a compound of Formula (II) comprising Formula (IIc) or a pharmaceutically acceptable salt thereof that is substantially free of Formula (IIb), in combination with sofosbuvir.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, sofosbuvir comprises the structure of Formula (III):

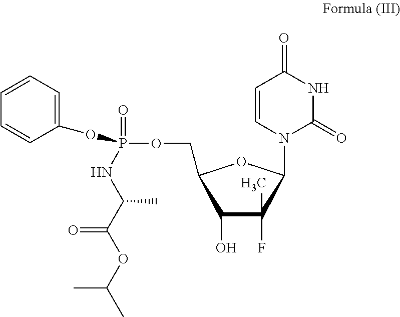

or a pharmaceutically acceptable salt thereof.

In some embodiments, the dosage of sofosbuvir is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of sofosbuvir is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of sofosbuvir is between about 50 mg and about 1000 mg. In some embodiments, the dosage of sofosbuvir is between about 100 mg and about 1000 mg. In some embodiments, the dosage of sofosbuvir is between about 100 mg and about 600 mg. In some embodiments, the dosage of sofosbuvir is between about 250 mg and about 500 mg. In some embodiments, the dosage of sofosbuvir is about 400 mg.

In some embodiments, in a method described herein, a compound of Formula (I) or Formula (II) is administered simultaneously with sofosbuvir. In some embodiments, a compound of Formula (I) or Formula (II) and sofosbuvir are administered within about 1 minute to about 48 hours of one another. In some embodiments, a compound of Formula (I) or Formula (II) and sofosbuvir are administered within about 1 minute, about 2 minutes, about 5 minutes, about 10 minutes, about 15 minutes, about 20 minutes, about 30 minutes, about 45 minutes, about 1 hour, about 1.5 hours, about 2 hours, about 3 hours, about 4 hours, about 5 hours, about 6 hours, about 8 hours, about 10 hours, about 12 hours, about 16 hours, about 20 hours, about 24 hours, about 36 hours, or about 48 hours of one another. In some embodiments, a compound of Formula (I) or Formula (II) and sofosbuvir are administered within about 5 minutes to about 12 hours of one another. In some embodiments, a compound of Formula (I) or Formula (II) and sofosbuvir are administered within about 5 minutes to about 6 hours of one another.

In some embodiments, in a method described herein, the combination of a compound of Formula (I) or Formula (II) and sofosbuvir is administered with food. In some embodiments, the administration of a compound of Formula (I) or Formula (II) and sofosbuvir occurs between about 30 minutes and about 6 hours after consumption of food by a subject. In some embodiments, the administration of a compound of Formula (I) or Formula (II) and sofosbuvir occurs between about 30 minutes and 60 minutes, about 90 minutes, about 2 hours, about 3 hours, about 4 hours, about 5 hours, or about 6 hours after consumption of food by a subject. In some embodiments, the combination of a compound of Formula (I) or Formula (II) and sofosbuvir is administered to a subject without food. In some embodiments, the combination of a compound of Formula (I) or Formula (II) and sofosbuvir is administered to a subject in the fasted state.

In some embodiments, in a method described herein, the combination of a compound of Formula (I) or Formula (II) and sofosbuvir has a synergistic or additive effect. In some embodiments, the combination of a compound of Formula (I) or Formula (II) and sofosbuvir has a synergistic effect. In some embodiments, the synergistic effect is dependent on the ratio of a compound of Formula (I) or Formula (II) to sofosbuvir administered to the subject. In some embodiments, the ratio of a compound of Formula (I) or Formula (II) to sofosbuvir is e.g., about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.25, about 1:2.5, about 1:3, about 1:4, about 1:5, about 1:6, about 1:7, about 1:8, about 1:9, about 1:10, about 1:12, about 1:15, or about 1:20, resulting in a synergistic effect. In some embodiments, the ratio of sofosbuvir to a compound of Formula (I) or Formula (II) is e.g., about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.25, about 1:2.5, about 1:3, about 1:4, about 1:5, about 1:6, about 1:7, about 1:8, about 1:9, about 1:10, about 1:12, about 1:15, or about 1:20, resulting in a synergistic effect. In some embodiments, the ratio of sofosbuvir to a compound of Formula (I) or Formula (II) is between about 2.5:1 to about 1:2.5, e.g., between about 1:1 to about 1:2.5, resulting in a synergistic effect.

In some embodiments, in a method described herein, the combination of a compound of Formula (II) and sofosbuvir has a synergistic or additive effect. In some embodiments, the combination of a compound of Formula (II) and sofosbuvir has a synergistic effect. In some embodiments, the synergistic effect is dependent on the ratio of a compound of Formula (II) to sofosbuvir administered to the subject. In some embodiments, the ratio of a compound of Formula (II) to sofosbuvir is e.g., about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.25, about 1:2.5, about 1:3, about 1:4, about 1:5, about 1:6, about 1:7, about 1:8, about 1:9, about 1:10, about 1:12, about 1:15, or about 1:20, resulting in a synergistic effect. In some embodiments, the ratio of sofosbuvir to a compound Formula (II) is e.g., about 1:1, about 1:1.1, about 1:1.2, about 1:1.3, about 1:1.4, about 1:1.5, about 1:1.6, about 1:1.7, about 1:1.8, about 1:1.9, about 1:2, about 1:2.25, about 1:2.5, about 1:3, about 1:4, about 1:5, about 1:6, about 1:7, about 1:8, about 1:9, about 1:10, about 1:12, about 1:15, or about 1:20, resulting in a synergistic effect. In some embodiments, the ratio of sofosbuvir to a compound of Formula (II) is between about 2.5:1 to about 1:2.5, e.g., between about 1:1 to about 1:2.5, resulting in a synergistic effect.

In some embodiments, in a method described herein, the combination of a compound of Formula (I) or Formula (II) and sofosbuvir has a synergistic effect, wherein the anti-HCV activity of one or both agents is greater than the sum of the anti-HCV activity observed with either agent alone. In some embodiments, the synergistic effect of a combination of a compound of Formula (I) or Formula (II) and sofosbuvir has an anti-HCV activity that is at least about 1.1, about 1.25, about 1.5, about 1.75, about 2, about 2.5, about 3, about 4, about 5, about 10, about 12.5, about 15, about 20, about 25, or about 50 times greater than the sum of the anti-HCV activity observed with either agent alone.

In some embodiments, in a method described herein, the combination of a compound of Formula (II) and sofosbuvir has a synergistic effect, wherein the anti-HCV activity of one or both agents is greater than the sum of the anti-HCV activity observed with either agent alone. In some embodiments, the synergistic effect of a combination of a compound of Formula (II) and sofosbuvir has an anti-HCV activity that is at least about 1.1, about 1.25, about 1.5, about 1.75, about 2, about 2.5, about 3, about 4, about 5, about 10, about 12.5, about 15, about 20, about 25, or about 50 times greater than the sum of the anti-HCV activity observed with either agent alone.

In some embodiments, the combination of a compound of Formula (I) or Formula (II) and sofosbuvir has an additive effect.

In some embodiments, the IC₅₀ value of sofosbuvir is reduced by an amount greater than or equal to about 1.5 fold when administered in combination with a compound of Formula (I) or Formula (II). In some embodiments, the IC₅₀ value of sofosbuvir is reduced by an amount greater than or equal to about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.75 fold, about 3.0 fold, about 3.5 fold, about 4.0 fold, about 4.5 fold, about 5.0 fold, or more when administered in combination with a compound of Formula (I) or Formula (II).

In some embodiments, the IC₅₀ value of sofosbuvir is reduced by an amount greater than or equal to about 1.5 fold when administered in combination with a compound of Formula (II). In some embodiments, the IC₅₀ value of sofosbuvir is reduced by an amount greater than or equal to about 1.6 fold, about 1.7 fold, about 1.8 fold, about 1.9 fold, about 2.0 fold, about 2.1 fold, about 2.2 fold, about 2.3 fold, about 2.4 fold, about 2.5 fold, about 2.75 fold, about 3.0 fold, about 3.5 fold, about 4.0 fold, about 4.5 fold, about 5.0 fold, or more when administered in combination with a compound of Formula (II).

In some embodiments, in a method described herein, the combination of a compound of Formula (I) or Formula (II) and sofosbuvir is administered orally. In some embodiments, the compound of Formula (I) or Formula (II) is administered orally. In some embodiments, sofosbuvir is administered orally. In some embodiments, the combination of a compound of Formula (I) or Formula (II) and sofosbuvir is formulated a liquid or solid dosage form. In some embodiments, the compound of Formula (I) or Formula (II) is formulated as a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, in a method described herein, the dosage of a combination of a compound of Formula (I) or Formula (II) and sofosbuvir is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of a combination of a compound of Formula (I) or Formula (II) and sofosbuvir is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of a combination of a compound of Formula (I) or Formula (II) and sofosbuvir is between about 50 mg and about 1000 mg. In some embodiments, the dosage of a combination of a compound of Formula (I) or Formula (II) and sofosbuvir is between about 200 mg and about 1000 mg.

In some embodiments, in a method described herein, a combination of a compound of Formula (I) or Formula (II) and sofosbuvir is administered daily. In some embodiments, a combination of a compound of Formula (I) or Formula (II) and sofosbuvir is administered once daily. In some embodiments, a combination of a compound of Formula (I) or Formula (II) and sofosbuvir is administered more than once a day, e.g., twice a day, three times a day, four times a day. In some embodiments, a combination of a compound of Formula (I) or Formula (II) and sofosbuvir is administered every other day, every 2 days, every 3 days, every 4 days, or more. In some embodiments, a combination of a compound of Formula (I) or Formula (II) and sofosbuvir is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, in a method described herein, a combination of a compound of Formula (I) or Formula (II) and sofosbuvir is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b). In some embodiments, the subject is infected with HCV genotype 2. In some embodiments, the subject is infected with HCV genotype 3.

In some embodiments, in a method described herein, the subject is infected with a resistant strain of HCV.

In some embodiments, in a method described herein, the subject is treatment naïve. In some embodiments, the subject has previously been treated for HCV infection.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir.

In another aspect, the present invention features a kit comprising a pharmaceutical composition comprising a compound of Formula (I), wherein the compound is selected from:

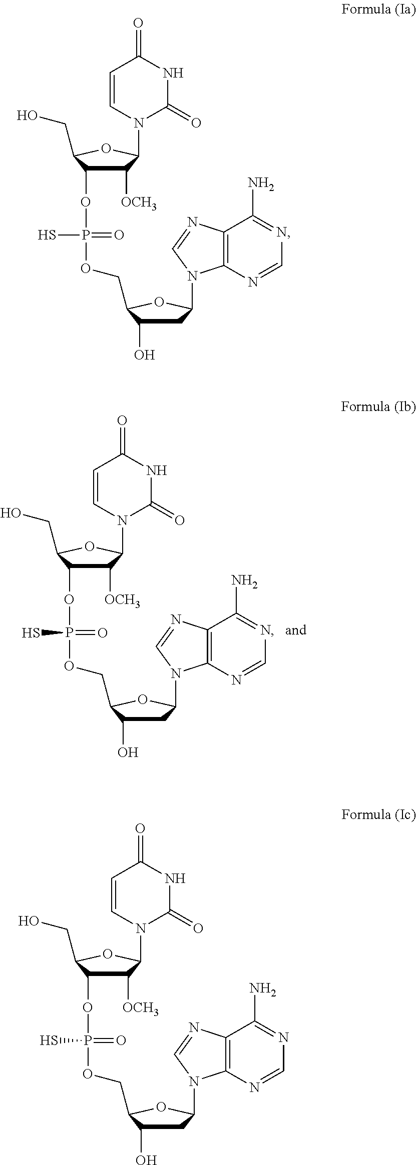

or a prodrug or pharmaceutically acceptable salt thereof in combination with sofosbuvir to thereby treat the subject. In some embodiments, the prodrug of Formula (I) is a compound of Formula (II), wherein the compound is selected from:

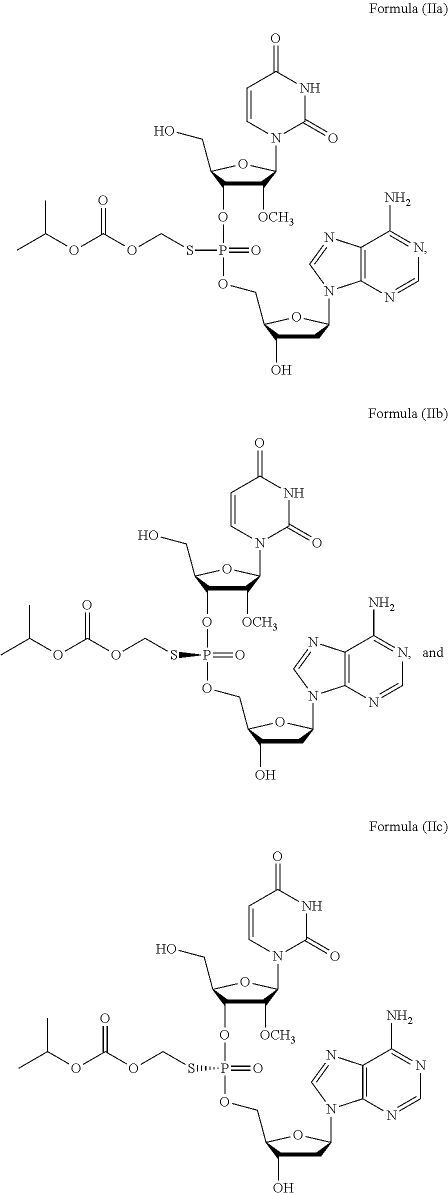

or a pharmaceutically acceptable salt thereof. In some embodiments, the composition comprises a compound of Formula (I) in combination with sofosbuvir. In some embodiments, the composition comprises a compound of Formula (II) in combination with sofosbuvir.

In some embodiments, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b). In some embodiments, the subject is infected with HCV genotype 2. In some embodiments, the subject is infected with HCV genotype 3.

In some embodiments, the subject is infected with a resistant strain of HCV.

In some embodiments, the subject is treatment naïve. In some embodiments, the subject has previously been treated for HCV infection.

In some embodiments, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, the kit further comprises an additional agent or treatment. In some embodiments, the additional agent or treatment is formulated in a composition with a compound of Formula (I) or Formula (II) and sofosbuvir. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir.

In another aspect, the present invention features a method of treating a subject infected with a drug-resistant strain of the Hepatitis C virus (HCV), the method comprising administering to the subject a compound of Formula (I), wherein the compound is selected from:

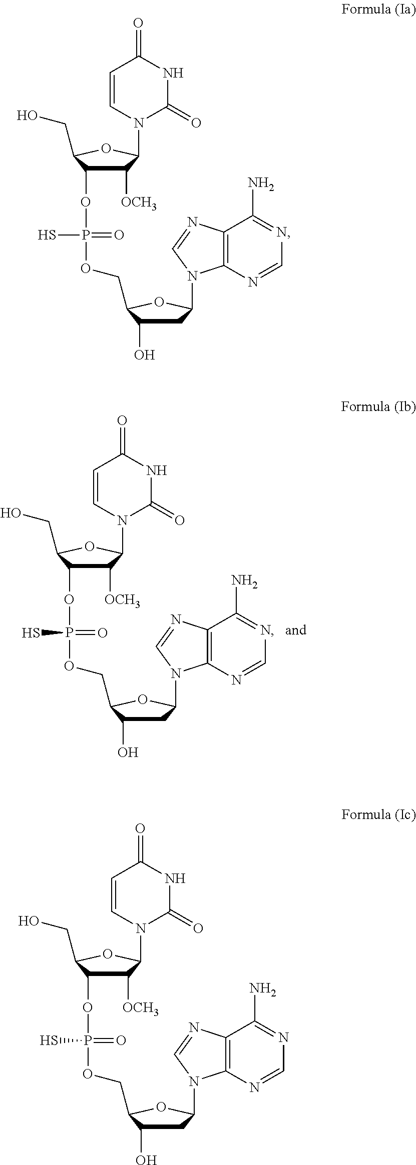

or a prodrug or pharmaceutically acceptable salt thereof to thereby treat the subject. In some embodiments, the prodrug of Formula (I) is a compound of Formula (II), wherein the compound is selected from:

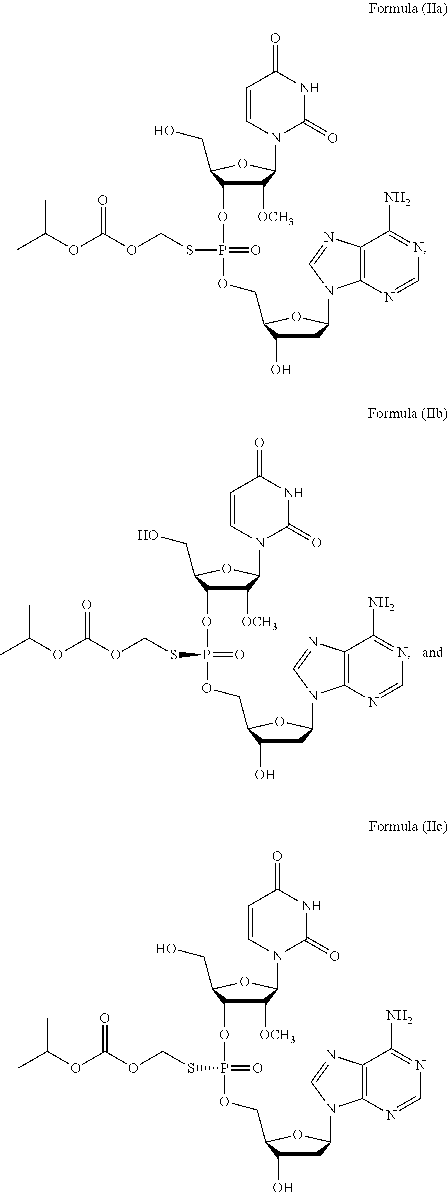

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof.

In some embodiments, the viral load of the drug-resistant strain of HCV is not substantially reduced by exposure to an anti-HCV agent other than a compound of Formula (I) or Formula (II) or a pharmaceutically acceptable salt thereof. In some embodiments, the viral load of the drug-resistant strain of HCV is reduced by less than about 50%, about 40%, about 30%, about 20%, about 15%, about 10%, about 5%, about 2.5%, about 1%, about 0.5%, about 0.1%, or less upon exposure to an anti-HCV agent other than a compound of Formula (I) or Formula (II) or a pharmaceutically acceptable salt thereof. In some embodiments, the viral load of the drug-resistant strain of HCV is reduced by less than about 2 log units, about 1.5 log units, about 1 log unit, about 0.5 log units, about 0.1 log units, or less upon administration of an anti-HCV agent other than a compound of Formula (I) or Formula (II) or a pharmaceutically acceptable salt thereof.

In some embodiments, the viral load of the drug-resistant strain of HCV is substantially reduced by a compound of Formula (I) or Formula (II) or a pharmaceutically acceptable salt thereof. In some embodiments, the viral load of the drug-resistant strain of HCV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HCV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof, and the anti-HCV agent is an interferon, a nucleoside analog, a non-nucleoside antiviral, a non-interferon immune enhancer, or a direct-acting antiviral agent. In some embodiments, the anti-HCV agent is sofosbuvir, interferon (e.g., peg-interferon), ribavirin, telaprevir, ledipasvir, danoprevir, ombitasvir, daclatsavir, dasabuvir, boceprevir, ciluprevir, paritaprevir, asunaprevir, tegobuvir, simeprevir, GS-9256, or a combination thereof.

In some embodiments, the drug-resistant HCV strain is an HCV variant strain or HCV mutant strain. In some embodiments, the drug-resistant HCV strain comprises a variant or mutant form of the E1, E2, NS1, NS2, NS3, NS4A, NS4B, NS5A, or NS5B proteins. In some embodiments, the drug-resistant HCV variant comprises an amino acid mutation (e.g., an amino acid substitution, addition, or deletion) in the sequence of the E1, E2, NS1, NS2, NS3, NS4A, NS4B, NS5A, or NS5B proteins, e.g., as compared with a reference sequence.

In some embodiments, the amino acid mutation (e.g., an amino acid substitution, addition, or deletion) in the N3 protein sequence comprises a mutation at amino acid positions 9, 16, 18, 23, 36, 39, 40, 41, 43, 54, 55, 65, 67, 70, 71, 80, 89, 109, 138, 155, 156, 162, 168, 170, 174, 176, 179, 260, or 489, e.g., as compared with a reference sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence. In some embodiments, the amino acid mutation (e.g., an amino acid substitution, addition, or deletion) in the NS5B protein sequence comprises a mutation at amino acid positions 15, 95, 96, 142, 152, 156, 222, 223, 244, 282, 309, 310, 320, 321, 326, 329, 333, 365, 411, 414, 415, 423, 445, 448, 451, 452, 495, 554, 558, or 559, e.g., as compared with a reference sequence.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, the compound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b), HCV genotype 2, HCV genotype 3, HCV genotype 4, HCV genotype 5, or HCV genotype 6. In some embodiments, the subject is infected with HCV genotype 1 (e.g., HCV-1a, HCV-1b). In some embodiments, the subject is infected with HCV genotype 2. In some embodiments, the subject is infected with HCV genotype 3 (e.g., HCV-3a, HCV-3b). In some embodiments, a compound of Formula (I) or Formula (II) has pan-genotypic activity.

In some embodiments, in a method described herein, the subject is treatment naïve. In some embodiments, the subject has previously been treated for HCV infection. In some embodiments, the previous treatment for HCV infection has failed. In some embodiments, the subject has relapsed.

In some embodiments, the subject has been previously been treated with an anti-HCV agent other than a compound of Formula (I) or Formula (II) or a pharmaceutically acceptable salt thereof (e.g., an interferon, ribavirin) and is suffering from a relapsed HCV infection.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

In another aspect, the present invention features a method of treating a subject infected with the Hepatitis C virus (HCV) that has previously been administered an anti-HCV agent, the method comprising administering to the subject a compound of Formula (I), wherein the compound is selected from:

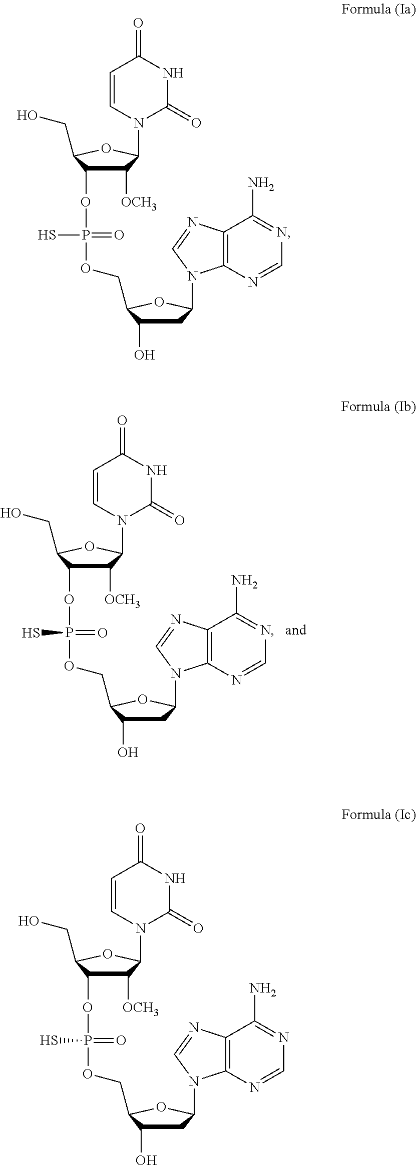

or a prodrug or pharmaceutically acceptable salt thereof to thereby treat the subject. In some embodiments, the prodrug of Formula (I) is a compound of Formula (II), wherein the compound is selected from:

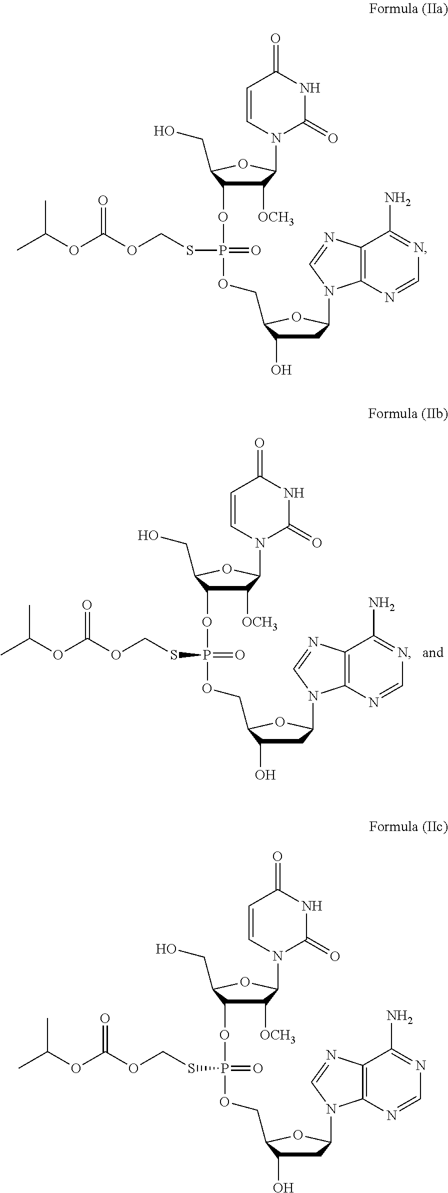

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the HCV strain is a drug-resistant HCV strain. In some embodiments, the drug-resistant strain HCV strain is resistant to an anti-HCV agent other than a compound of Formula (I) or Formula (II) or a pharmaceutically acceptable salt thereof.

In some embodiments, the viral load of the drug-resistant strain of HCV is not substantially reduced by exposure to an anti-HCV agent other than a compound of Formula (I) or Formula (II) or a pharmaceutically acceptable salt thereof. In some embodiments, the viral load of the drug-resistant strain of HCV is reduced by less than about 50%, about 40%, about 30%, about 20%, about 15%, about 10%, about 5%, about 2.5%, about 1%, about 0.5%, about 0.1%, or less upon exposure to an anti-HCV agent other than a compound of Formula (I) or Formula (II) or a pharmaceutically acceptable salt thereof. In some embodiments, the viral load of the drug-resistant strain of HCV is reduced by less than about 2 log units, about 1.5 log units, about 1 log unit, about 0.5 log units, about 0.1 log units, or less upon administration of an anti-HCV agent other than a compound of Formula (I) or Formula (II) or a pharmaceutically acceptable salt thereof.

In some embodiments, the viral load of the drug-resistant strain of HCV is substantially reduced by a compound of Formula (I) or Formula (II) or a pharmaceutically acceptable salt thereof. In some embodiments, the viral load of the drug-resistant strain of HCV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HCV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof, and the anti-HCV agent is an interferon, a nucleoside analog, a non-nucleoside antiviral, a non-interferon immune enhancer, or a direct-acting antiviral agent. In some embodiments, the anti-HCV agent is sofosbuvir, interferon (e.g., peg-interferon), ribavirin, telaprevir, ledipasvir, danoprevir, ombitasvir, daclatsavir, dasabuvir, boceprevir, ciluprevir, paritaprevir, asunaprevir, tegobuvir, simeprevir, GS-9256, or a combination thereof.

In some embodiments, the drug-resistant HCV strain is an HCV variant strain or HCV mutant strain. In some embodiments, the drug-resistant HCV strain comprises a variant or mutant form of the E1, E2, NS1, NS2, NS3, NS4A, NS4B, NS5A, or NS5B proteins. In some embodiments, the drug-resistant HCV variant comprises an amino acid mutation (e.g., an amino acid substitution, addition, or deletion) in the sequence of the E1, E2, NS1, NS2, NS3, NS4A, NS4B, NS5A, or NS5B proteins, e.g., as compared with a reference sequence.

In some embodiments, the amino acid mutation (e.g., an amino acid substitution, addition, or deletion) in the N3 protein sequence comprises a mutation at amino acid positions 9, 16, 18, 23, 36, 39, 40, 41, 43, 54, 55, 65, 67, 70, 71, 80, 89, 109, 138, 155, 156, 162, 168, 170, 174, 176, 179, 260, or 489, e.g., as compared with a reference sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence. In some embodiments, the amino acid mutation (e.g., an amino acid substitution, addition, or deletion) in the NS5B protein sequence comprises a mutation at amino acid positions 15, 95, 96, 142, 152, 156, 222, 223, 244, 282, 309, 310, 320, 321, 326, 329, 333, 365, 411, 414, 415, 423, 445, 448, 451, 452, 495, 554, 558, or 559, e.g., as compared with a reference sequence.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, the compound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b), HCV genotype 2, HCV genotype 3, HCV genotype 4, HCV genotype 5, or HCV genotype 6. In some embodiments, the subject is infected with HCV genotype 1 (e.g., HCV-1a, HCV-1b). In some embodiments, the subject is infected with HCV genotype 2. In some embodiments, the subject is infected with HCV genotype 3 (e.g., HCV-3a, HCV-3b). In some embodiments, a compound of Formula (I) or Formula (II) has pan-genotypic activity.

In some embodiments, in a method described herein, the subject is treatment naïve. In some embodiments, the subject has previously been treated for HCV infection. In some embodiments, the previous treatment for HCV infection has failed. In some embodiments, the subject has relapsed.

In some embodiments, the subject has been previously been treated with an anti-HCV agent other than a compound of Formula (I) or Formula (II) or a pharmaceutically acceptable salt thereof (e.g., an interferon, ribavirin) and is suffering from a relapsed HCV infection.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

In another aspect, the present invention features a method of treating a subject infected with a drug-resistant strain of the Hepatitis C virus (HCV) comprising a variant or mutant form of the NS5A protein, the method comprising administering to the subject a compound of Formula (I), wherein the compound is selected from:

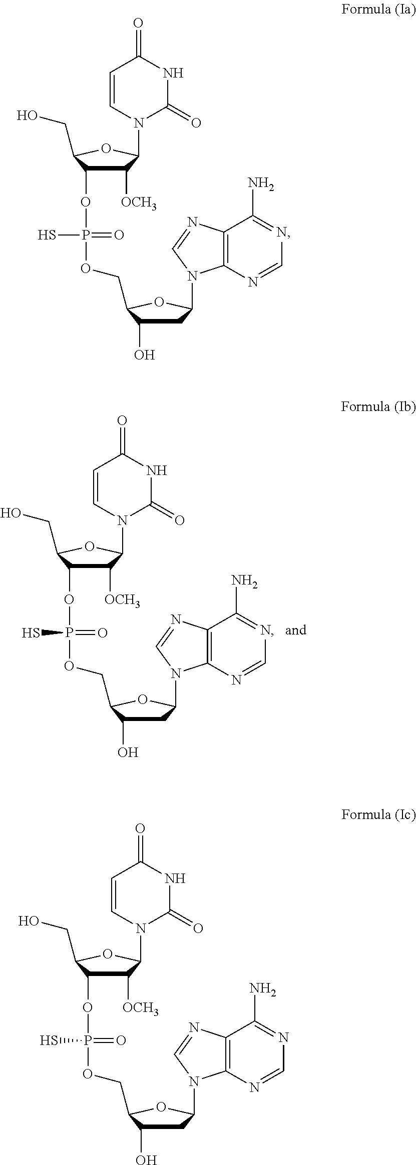

or a prodrug or pharmaceutically acceptable salt thereof to thereby treat the subject. In some embodiments, the prodrug of Formula (I) is a compound of Formula (II), wherein the compound is selected from:

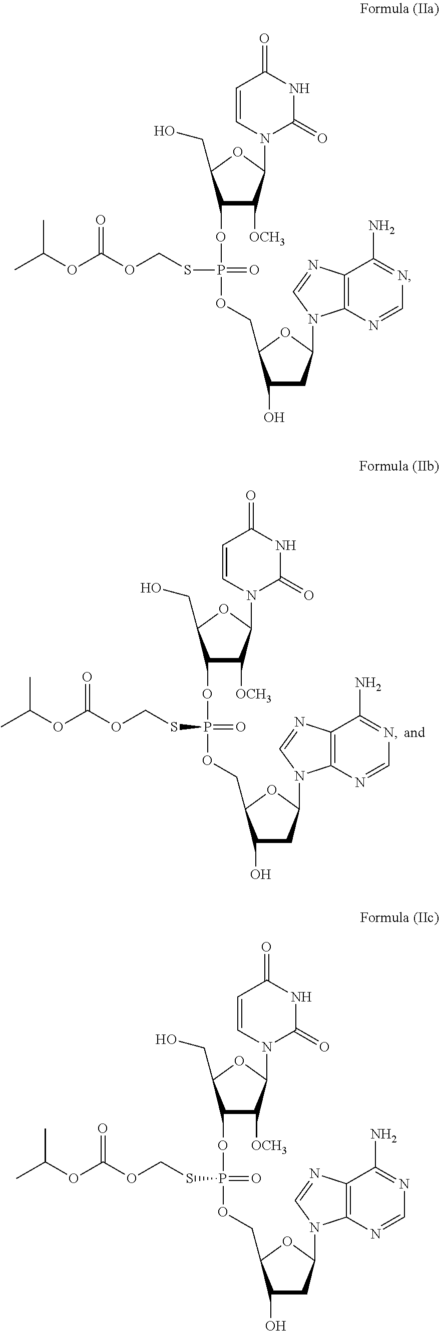

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof.

In some embodiments, the viral load of the drug-resistant strain of HCV is not substantially reduced by exposure to an anti-HCV agent other than a compound of Formula (I) or Formula (II) or a pharmaceutically acceptable salt thereof. In some embodiments, the viral load of the drug-resistant strain of HCV is reduced by less than about 50%, about 40%, about 30%, about 20%, about 15%, about 10%, about 5%, about 2.5%, about 1%, about 0.5%, about 0.1%, or less upon exposure to an anti-HCV agent other than a compound of Formula (I) or Formula (II) or a pharmaceutically acceptable salt thereof. In some embodiments, the viral load of the drug-resistant strain of HCV is reduced by less than about 2 log units, about 1.5 log units, about 1 log unit, about 0.5 log units, about 0.1 log units, or less upon administration of an anti-HCV agent other than a compound of Formula (I) or Formula (II) or a pharmaceutically acceptable salt thereof.

In some embodiments, the viral load of the drug-resistant strain of HCV is substantially reduced by a compound of Formula (I) or Formula (II) or a pharmaceutically acceptable salt thereof. In some embodiments, the viral load of the drug-resistant strain of HCV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HCV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof, and the anti-HCV agent is an interferon, a nucleoside analog, a non-nucleoside antiviral, a non-interferon immune enhancer, or a direct-acting antiviral agent. In some embodiments, the anti-HCV agent is sofosbuvir, interferon (e.g., peg-interferon), ribavirin, telaprevir, ledipasvir, danoprevir, ombitasvir, daclatsavir, dasabuvir, boceprevir, ciluprevir, paritaprevir, asunaprevir, tegobuvir, simeprevir, GS-9256, or a combination thereof.

In some embodiments, the drug-resistant HCV strain is an HCV variant strain or HCV mutant strain. In some embodiments, the drug-resistant HCV variant comprises an amino acid mutation (e.g., an amino acid substitution, addition, or deletion) in the sequence of the NS5A protein, e.g., as compared with a reference sequence.

In some embodiments, the amino acid mutation (e.g., an amino acid substitution, addition, or deletion) in the NS5A protein sequence comprises a mutation between amino acids 1 and 447, e.g., as compared to a reference or consensus sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence. In some embodiments, the amino acid mutation (e.g., an amino acid substitution, addition, or deletion) in the NS5A protein sequence comprises a mutation at amino acid positions 23, 24, 28, 30, 31, 32, 37, 54, 58, 63, 93, 295, 318, 320, 356, 404, or 442, e.g., as compared to a reference or consensus sequence. In some embodiments, the amino acid mutation (e.g., an amino acid substitution, addition, or deletion) in the NS5A protein sequence comprises a mutation at amino acid positions 1, 8, 26, 30, 31, 32, 34, 37, 44, 46, 48, 58, 64, 78, 85, 90, 99, 107, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 326, 346, 347, 348, 349, 367, 368, 370, 388, 390, 392, 393, 395, 397, 399, 400, 401, 403, 404, 405, 410, 413, 439, 441, or 442, e.g., as compared to a reference or consensus sequence.

In some embodiments, the amino acid mutation is an amino acid substitution, e.g., as compared to a reference or consensus sequence. In some embodiments, the amino acid mutation is an amino acid addition, e.g., as compared to a reference or consensus sequence. In some embodiments, the amino acid mutation is an amino acid deletion, e.g., as compared to a reference or consensus sequence.

In some embodiments, the amino acid mutation in the NS5A protein sequence comprises a mutation at amino acid position 31 or 93, e.g., as compared to a reference or consensus sequence. In some embodiments, the amino acid mutation in the NS5A protein sequence comprises a mutation at amino acid position 31, e.g., as compared to a reference or consensus sequence. In some embodiments, the amino acid mutation in the NS5A protein sequence comprises a mutation at amino acid position 93, e.g., as compared to a reference or consensus sequence. In some embodiments, the amino acid mutation in the NS5A protein sequence comprises mutations at amino acid positions 31 and 93, e.g., as compared to a reference or consensus sequence. In some embodiments, the amino acid mutation in the NS5A protein sequence comprises a L31F, L31H, L31I, L31P, L31R, or L31V mutation, e.g., as compared to a reference or consensus sequence. In some embodiments, the amino acid mutation in the NS5A protein sequence comprises a Y93C, Y93D, Y93E, Y93F, Y93G, Y93H, Y93K, Y93L, Y93N, Y93P, Y93Q, Y93R, Y93S, or Y93T mutation, e.g., as compared to a reference or consensus sequence.

In some embodiments, the drug-resistant HCV strain comprises more than one amino acid mutation (e.g., an amino acid substitution, addition, or deletion) to the sequence of the NS5A protein, e.g., as compared to a reference or consensus sequence, e.g., more than 2, more than 3, more than 4, more than 5, more than 6, more than 7, more than 8, more than 9, more than 10, more than 12, more than 15, more than 20, more than 25, more than 30 amino acid mutations.

In some embodiments, the drug-resistant HCV strain further comprises a variant or mutant form of the E1, E2, NS1, NS2, NS3, NS4A, NS4B, or NS5B proteins.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally.

In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, the compound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b), HCV genotype 2, HCV genotype 3, HCV genotype 4, HCV genotype 5, or HCV genotype 6. In some embodiments, the subject is infected with HCV genotype 1 (e.g., HCV-1a, HCV-1b). In some embodiments, the subject is infected with HCV genotype 2. In some embodiments, the subject is infected with HCV genotype 3 (e.g., HCV-3a, HCV-3b). In some embodiments, a compound of Formula (I) or Formula (II) has pan-genotypic activity.

In some embodiments, in a method described herein, the subject is treatment naïve. In some embodiments, the subject has previously been treated for HCV infection. In some embodiments, the previous treatment for HCV infection has failed. In some embodiments, the subject has relapsed.

In some embodiments, the subject has been previously been treated with an anti-HCV agent other than a compound of Formula (I) or Formula (II) or a pharmaceutically acceptable salt thereof (e.g., an interferon, ribavirin) and is suffering from a relapsed HCV infection.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

In another aspect, the present invention features a method of treating a subject infected with the Hepatitis C virus (HCV) comprising a variant or mutant form of the NS5A protein, wherein the subject has previously been administered an anti-HCV agent and the method comprises administering to the subject a compound of Formula (I), wherein the compound is selected from:

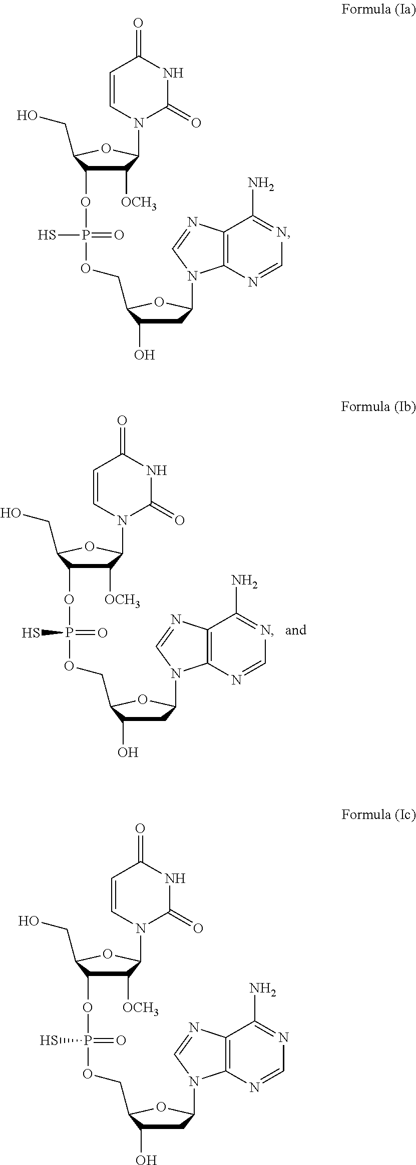

or a prodrug or pharmaceutically acceptable salt thereof to thereby treat the subject. In some embodiments, the prodrug of Formula (I) is a compound of Formula (II), wherein the compound is selected from:

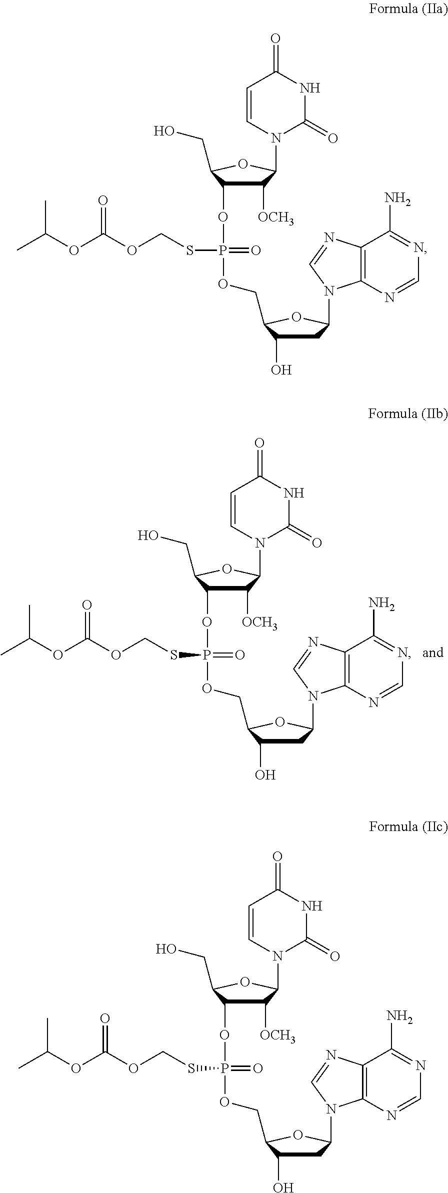

or a pharmaceutically acceptable salt thereof. In some embodiments, the method comprises administering to the subject a compound of Formula (I) or a pharmaceutically acceptable salt thereof. In some embodiments, the method comprises administering to the subject a compound of Formula (II) or a pharmaceutically acceptable salt thereof.

In some embodiments, the HCV strain is a drug-resistant HCV strain. In some embodiments, the drug-resistant strain HCV strain is resistant to an anti-HCV agent other than a compound of Formula (I) or Formula (II) or a pharmaceutically acceptable salt thereof.

In some embodiments, the viral load of the drug-resistant strain of HCV is not substantially reduced by exposure to an anti-HCV agent other than a compound of Formula (I) or Formula (II) or a pharmaceutically acceptable salt thereof. In some embodiments, the viral load of the drug-resistant strain of HCV is reduced by less than about 50%, about 40%, about 30%, about 20%, about 15%, about 10%, about 5%, about 2.5%, about 1%, about 0.5%, about 0.1%, or less upon exposure to an anti-HCV agent other than a compound of Formula (I) or Formula (II) or a pharmaceutically acceptable salt thereof. In some embodiments, the viral load of the drug-resistant strain of HCV is reduced by less than about 2 log units, about 1.5 log units, about 1 log unit, about 0.5 log units, about 0.1 log units, or less upon administration of an anti-HCV agent other than a compound of Formula (I) or Formula (II) or a pharmaceutically acceptable salt thereof.

In some embodiments, the viral load of the drug-resistant strain of HCV is substantially reduced by a compound of Formula (I) or Formula (II) or a pharmaceutically acceptable salt thereof. In some embodiments, the viral load of the drug-resistant strain of HCV is reduced by more than about 10%, about 20%, about 30%, about 40%, about 50%, about 60%, about 70%, about 80%, about 90%, about 95%, about 99%, about 99.9%, or about 99.99% or more upon administration of a compound of Formula (I) or Formula (II) or a pharmaceutically acceptable salt thereof. In some embodiments, the viral load of the drug-resistant strain of HCV is reduced by more than about 1 log unit, about 1.5 log units, about 2 log units, about 2.5 log units, about 3 log units, about 3.5 log units, about 4 log units, about 4.5 log units, about 5 log units, or more upon administration to a compound of Formula (I) or Formula (II) or a pharmaceutically acceptable salt thereof.

In some embodiments, the drug-resistant strain of HCV is resistant to an anti-HCV agent other than a compound of Formula (I) or Formula (II) or a pharmaceutically acceptable salt thereof, and the anti-HCV agent is an interferon, a nucleoside analog, a non-nucleoside antiviral, a non-interferon immune enhancer, or a direct-acting antiviral agent. In some embodiments, the anti-HCV agent is sofosbuvir, interferon (e.g., peg-interferon), ribavirin, telaprevir, ledipasvir, danoprevir, ombitasvir, daclatsavir, dasabuvir, boceprevir, ciluprevir, paritaprevir, asunaprevir, tegobuvir, simeprevir, GS-9256, or a combination thereof.

In some embodiments, the drug-resistant HCV strain is an HCV variant strain or HCV mutant strain. In some embodiments, the drug-resistant HCV variant comprises an amino acid mutation (e.g., an amino acid substitution, addition, or deletion) in the sequence of the NS5A protein, e.g., as compared with a reference sequence.

In some embodiments, the amino acid mutation (e.g., an amino acid substitution, addition, or deletion) in the NS5A protein sequence comprises a mutation between amino acids 1 and 447, e.g., as compared to a reference or consensus sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence. In some embodiments, the amino acid mutation (e.g., an amino acid substitution, addition, or deletion) in the NS5A protein sequence comprises a mutation at amino acid positions 23, 24, 28, 30, 31, 32, 37, 54, 58, 63, 93, 295, 318, 320, 356, 404, or 442, e.g., as compared to a reference or consensus sequence. In some embodiments, the amino acid mutation (e.g., an amino acid substitution, addition, or deletion) in the NS5A protein sequence comprises a mutation at amino acid positions 1, 8, 26, 30, 31, 32, 34, 37, 44, 46, 48, 58, 64, 78, 85, 90, 99, 107, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 326, 346, 347, 348, 349, 367, 368, 370, 388, 390, 392, 393, 395, 397, 399, 400, 401, 403, 404, 405, 410, 413, 439, 441, or 442, e.g., as compared to a reference or consensus sequence.

In some embodiments, the amino acid mutation is an amino acid substitution, e.g., as compared to a reference or consensus sequence. In some embodiments, the amino acid mutation is an amino acid addition, e.g., as compared to a reference or consensus sequence. In some embodiments, the amino acid mutation is an amino acid deletion, e.g., as compared to a reference or consensus sequence.

In some embodiments, the amino acid mutation in the NS5A protein sequence comprises a mutation at amino acid position 31 or 93, e.g., as compared to a reference or consensus sequence. In some embodiments, the amino acid mutation in the NS5A protein sequence comprises a mutation at amino acid position 31, e.g., as compared to a reference or consensus sequence. In some embodiments, the amino acid mutation in the NS5A protein sequence comprises a mutation at amino acid position 93, e.g., as compared to a reference or consensus sequence. In some embodiments, the amino acid mutation in the NS5A protein sequence comprises mutations at amino acid positions 31 and 93, e.g., as compared to a reference or consensus sequence. In some embodiments, the amino acid mutation in the NS5A protein sequence comprises a L31F, L31H, L31I, L31P, L31R, or L31V mutation, e.g., as compared to a reference or consensus sequence. In some embodiments, the amino acid mutation in the NS5A protein sequence comprises a Y93C, Y93D, Y93E, Y93F, Y93G, Y93H, Y93K, Y93L, Y93N, Y93P, Y93Q, Y93R, Y93S, or Y93T mutation, e.g., as compared to a reference or consensus sequence.

In some embodiments, the drug-resistant HCV strain comprises more than one amino acid mutation (e.g., an amino acid substitution, addition, or deletion) to the sequence of the NS5A protein, e.g., as compared to a reference or consensus sequence, e.g., more than 2, more than 3, more than 4, more than 5, more than 6, more than 7, more than 8, more than 9, more than 10, more than 12, more than 15, more than 20, more than 25, more than 30 amino acid mutations.

In some embodiments, the drug-resistant HCV strain further comprises a variant or mutant form of the E1, E2, NS1, NS2, NS3, NS4A, NS4B, or NS5B proteins.

In some embodiments, the method described herein comprises administering to the subject a mixture of compounds of Formula (I) or pharmaceutically acceptable salts thereof. In some embodiments, the method described herein comprises administering to the subject a mixture of compounds of Formula (Ib) and Formula (Ic) or pharmaceutically acceptable salts thereof. In some embodiments, the mixture comprises a ratio of Formula (Ib) to Formula (Ic) of about 1:1 (e.g., a racemic mixture). In some embodiments, the mixture comprises a ratio of Formula (Ib) to Formula (Ic) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (Ic) to Formula (Ib) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (I) comprising Formula (Ib) and less than about 5% of Formula (Ic), e.g., less than about 4%, less than about 3%, less than about 2%, less than about 1%, less than about 0.5%, or less than about 0.1% of Formula (Ic). In some embodiments, the method described herein comprises administering to the subject a compound of Formula (I) comprising Formula (Ib) or a pharmaceutically acceptable salt thereof that is substantially free of Formula (Ic). In some embodiments, the method described herein comprises administering to the subject a mixture of compounds of Formula (I) comprising Formula (Ic) and less than about 5% of Formula (Ib), e.g., less than about 4%, less than about 3%, less than about 2%, less than about 1%, less than about 0.5%, or less than about 0.1% of Formula (Ib). In some embodiments, the method described herein comprises administering to the subject a compound of Formula (I) comprising Formula (Ic) or a pharmaceutically acceptable salt thereof that is substantially free of Formula (Ib).

In some embodiments, the method described herein comprises administering to the subject a mixture of compounds of Formula (II) or pharmaceutically acceptable salts thereof. In some embodiments, the method described herein comprises administering to the subject a mixture of Formula (IIb) and Formula (IIc) or pharmaceutically acceptable salts thereof. In some embodiments, the mixture comprises a ratio of Formula (IIb) to Formula (IIc) of about 1:1 (e.g., a racemic mixture). In some embodiments, the mixture comprises a ratio of Formula (IIb) to Formula (IIc) of about 51:49, about 52:48, about 53:47, about 54:46, about 55:45, about 60:40, about 65:35, about 70:30, about 75:25, about 80:20, about 85:15, about 90:10, about 95:5, or about 99:1. In some embodiments, the mixture comprises a ratio of Formula (IIc) to Formula (IIb) of about 51:49, about 52:48, about 53:47, about 54:46, about 55:45, about 60:40, about 65:35, about 70:30, about 75:25, about 80:20, about 85:15, about 90:10, about 95:5, or about 99:1.

In some embodiments, the method described herein comprises administering to the subject a mixture of compounds of Formula (II) comprising Formula (IIb) and less than about 5% of Formula (IIc), e.g., less than about 4%, less than about 3%, less than about 2%, less than about 1%, less than about 0.5%, or less than about 0.1% of Formula (IIc). In some embodiments, the method described herein comprises administering to the subject a compound of Formula (II) comprising Formula (IIb) or a pharmaceutically acceptable salt thereof that is substantially free of Formula (IIc). In some embodiments, the method described herein comprises administering to the subject a mixture of compounds of Formula (II) comprising Formula (IIc) and less than about 5% of Formula (IIb), e.g., less than about 4%, less than about 3%, less than about 2%, less than about 1%, less than about 0.5%, or less than about 0.1% of Formula (IIb). In some embodiments, the method described herein comprises administering to the subject a compound of Formula (II) comprising Formula (IIc) or a pharmaceutically acceptable salt thereof that is substantially free of Formula (IIb).

In some embodiments, in a method described herein, the IC₅₀ value of a compound of Formula (I) or Formula (II) is less than 10 μM (e.g., a compound of Formula (II) is less than 10 μM). In some embodiments, the IC₅₀ value of a compound of Formula (I) or Formula (II) is less than 1 μM (e.g., a compound of Formula (II) is less than 1 μM). In some embodiments, the IC₅₀ value of a compound of Formula (I) or Formula (II) is less than 0.1 μM (e.g., the IC₅₀ value of a compound of Formula (II) is less than 0.1 μM). In some embodiments, the IC₅₀ value of a compound of Formula (I) or Formula (II) is less than 0.01 μM (e.g., the IC₅₀ value of a compound of Formula (II) is less than 0.1 μM).

In some embodiments, in a method described herein, the compound of Formula (I) or Formula (II) is administered orally. In some embodiments, the compound of Formula (I) is administered orally. In some embodiments, the compound of Formula (II) is administered orally. In some embodiments, the compound of Formula (I) or Formula (II) is administered parenterally. In some embodiments, the compound of Formula (I) is administered parenterally. In some embodiments, the compound of Formula (II) is administered parenterally. In some embodiments, the compound of Formula (I) or Formula (II) is administered intravenously. In some embodiments, the compound of Formula (I) is administered intravenously. In some embodiments, the compound of Formula (II) is administered intravenously.

In some embodiments, the compound of Formula (I) or Formula (II) is formulated a liquid or solid dosage form. In some embodiments, the liquid dosage form comprises a suspension, a solution, a linctus, an emulsion, a drink, an elixir, or a syrup. In some embodiments, the solid dosage form comprises a capsule, tablet, pill, dragée, powder, or microencapsulated dose form.

In some embodiments, the dosage of Formula (I) or Formula (II) is between about 10 mg and about 1500 mg, about 1250 mg, about 1000 mg, about 900 mg, about 800 mg, about 700 mg, about 600 mg, about 500 mg, about 400 mg, about 300 mg, about 250 mg, about 200 mg, about 150 mg, about 100 mg, about 75 mg, about 50 mg, about 25 mg, or less. In some embodiments, the dosage of Formula (I) or Formula (II) is between about 10 mg, about 25 mg, about 50 mg, about 75 mg, about 100 mg, about 150 mg, about 200 mg, about 250 mg, about 300 mg, about 400 mg, about 500 mg, about 600 mg, about 700 mg, about 800 mg, about 900 mg, about 1000 mg, about 1250 mg, and about 1500 mg. In some embodiments, the dosage of Formula (I) or Formula (II) is between about 50 mg and about 1000 mg. In some embodiments, the dosage of Formula (I) or Formula (II) is between about 200 mg and about 1000 mg.

In some embodiments, in a method described herein, the compound of Formula (I) or Formula (II) is administered daily. In some embodiments, the compound of Formula (I) or Formula (II) is administered once daily. In some embodiments, the compound of Formula (I) or Formula (II) is administered more than once a day, e.g., twice a day, three times a day, four times a day. In some embodiments, the compound of Formula (I) or Formula (II) is administered every other day, every 2 days, every 3 days, every 4 days, or more. In some embodiments, the compound of Formula (I) or Formula (II) is administered once a week, twice a week, three times a week, four times a week, five times a week, or six times a week.

In some embodiments, in a method described herein, the duration of the method is one day. In some embodiments, the duration of the method is greater than 1 day, e.g., about 2 days, about 3 days, about 4 days, about 5 days, about 6 days, about 7 days, about 8 days, about 9 days, about 10 days, about 11 days, about 12 days, about 13 days, about 14 days, about 2 weeks, about 3 weeks, about 4 weeks, about 1 month, about 1.5 months, about 2 months, about 3 months, about 4 months, about 5 months, about 6 months. In some embodiments, the duration of the method is between about 1 day and about 2 weeks. In some embodiments, the duration of the method is between 6 days and 14 days. In some embodiments, the duration of the method is for one week. In some embodiments, the duration of the method lasts until the subject is cured of HCV infection (e.g., until the subject presents an undetectable level of HCV RNA).

In some embodiments, in a method described herein, a compound of Formula (I) or Formula (II) is formulated as a pharmaceutical composition. In some embodiments, the pharmaceutical composition further comprises a pharmaceutically acceptable carrier or excipient.

In some embodiments, in a method described herein, the subject is a mammal. In some embodiments, the subject is a human. In some embodiments, the subject has been diagnosed with HCV infection. In some embodiments, the subject is diagnosed with chronic hepatitis C (CHC). In some embodiments, the genotype of the HCV infection is known. In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b), HCV genotype 2, HCV genotype 3, HCV genotype 4, HCV genotype 5, or HCV genotype 6. In some embodiments, the subject is infected with HCV genotype 1 (e.g., HCV-1a, HCV-1b). In some embodiments, the subject is infected with HCV genotype 2. In some embodiments, the subject is infected with HCV genotype 3 (e.g., HCV-3a, HCV-3b). In some embodiments, a compound of Formula (I) or Formula (II) has pan-genotypic activity.

In some embodiments, in a method described herein, the subject is treatment naïve. In some embodiments, the subject has previously been treated for HCV infection. In some embodiments, the previous treatment for HCV infection has failed. In some embodiments, the subject has relapsed.

In some embodiments, the subject has been previously been treated with an anti-HCV agent other than a compound of Formula (I) or Formula (II) or a pharmaceutically acceptable salt thereof (e.g., an interferon, ribavirin) and is suffering from a relapsed HCV infection.

In some embodiments, in a method described herein, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation. In some embodiments, the subject is non-cirrhotic.

In some embodiments, in a method described herein, the subject has been further diagnosed with an HIV infection. In some embodiments, the strain of HIV infection is known. In some embodiments, the subject is infected with HIV-1 or HIV-2 (e.g., strain 1 or strain 2).

In some embodiments, in a method described herein, the subject is further administered an additional agent or treatment or a pharmaceutically acceptable salt thereof. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

### BRIEF DESCRIPTION OF THE DRAWINGS

FIG. 1 depicts a series of graphs summarizing the mean plasma concentrations of Formula (IIa) over time after a single oral administration of Formula (IIa) under fasting and fed conditions for varying dosage amounts (100 mg, 200 mg, 400 mg, and 800 mg).

FIG. 2 depicts a series of graphs summarizing the mean plasma concentrations of the Sp-isomer (e.g., Formula (Ic)) over time after a single oral administration of Formula (IIa) under fasting and fed conditions for varying dosage amounts (100 mg, 200 mg, 400 mg, and 800 mg).

FIG. 3 depicts a series of graphs summarizing the mean plasma concentrations of the Rp-isomer (e.g., Formula (Ib)) over time after a single oral administration of Formula (IIa) under fasting and fed conditions for varying dosage amounts (100 mg, 200 mg, 400 mg, and 800 mg).

FIG. 4 depicts a series of graphs summarizing the mean (+SD) plasma concentrations of Formula (IIa) over time after single and multiple once daily oral administrations of Formula (IIa) under fasted conditions for varying dosage amounts (200 mg, 400 mg, and 900 mg).

FIG. 5 depicts a graph summarizing the mean (+SD) plasma trough concentrations of Formula (IIa) vs. time after multiple once daily oral administration of Formula (IIa) under fasted conditions.

FIG. 6 depicts a series of graphs summarizing the mean (+SD) plasma concentrations of the Sp-isomer (e.g., Formula (Ic)) vs. time after single and multiple once daily oral administration of Formula (IIa) under fasted conditions.

FIG. 7 depicts a graph summarizing the mean (+SD) plasma trough concentrations of the Sp-isomer (e.g., Formula (Ic)) vs. time after multiple once daily oral administration of Formula (IIa) under fasted conditions.

FIG. 8 depicts a series of graphs summarizing the mean (+SD) plasma concentrations of the Rp-isomer (e.g., Formula (IIb)) vs. time after single and multiple once daily oral administration of Formula (IIa) under fasted conditions.

FIG. 9 depicts a graph summarizing the mean (+SD) plasma trough concentrations of the Rp-isomer (e.g., Formula (IIb)) vs. time after multiple once daily oral administration of Formula (IIa) under fasted conditions.

FIG. 10 depicts a graph summarizing the maximum suppression of HCV RNA (Δ log HCV RNAₘₐₓ) of Formula (IIa) vs. plasma Formula (IIa) Cₘₐₓ on Day 7 of the MAD study.

FIGS. 11A-D depict a series of graphs summarizing the IC₅₀ determination of Formula (IIa) (FIG. 11A), sofosbuvir (FIG. 11B), and Formula (IIa) in combination with sofosbuvir (FIG. 11C) against cells infected with HCV. FIG. 11D shows that the combination of sofosbuvir and Formula (IIa) exhibits a synergistic effect on HCV-infected cells.

FIGS. 12A-B depict graphs summarizing the IC₅₀ determination of Formula (IIa) in serum samples taken from two patients infected with interferon (IFN) resistant strains of HCV. The IC₅₀ of Formula (IIa) was found to be 24 nM in a sample taken from patient 22 that suffered repeated relapse of HCV infection after combination interferon and ribvarin therapy (FIG. 12A). FIG. 12B depicts the IC₅₀ determination of Formula (IIa) (5.46 nM) in a sample taken from patient 23 that suffered from a relapse of HCV infection. A summary of the samples taken from representative patients is outlined in FIG. 16.

FIGS. 13A-C depict graphs summarizing the IC₅₀ determination of sofosbuvir (FIG. 13A), ribavirin (FIG. 13B), and Formula (IIa) (FIG. 13C, 4 nM) in serum samples taken from a patient (patient 30) infected with a sofosbuvir resistant strain of HCV. A summary of the samples taken from representative patients is outlined in FIG. 16.

FIGS. 14A-B depict graphs summarizing the IC₅₀ determination of Formula (IIa) in samples taken from two patients infected with telaprevir resistant strains of HCV. FIG. 14A depicts the IC₅₀ determination of Formula (IIa) (450 nM) in a sample taken from patient 7 prior to administration of telaprevir, while FIG. 14B shows the IC₅₀ of Formula (IIa) (6.4 nM) in a sample from patient 25 after treatment with telaprevir.

FIGS. 15A-C depicts charts comparing the IC₅₀ of Formula (IIa) in samples taken from patients that are treatment naïve vs. patients that have been previously treated for HCV infection. FIG. 15A compares the IC₅₀ of Formula (IIa) in patients by HCV genotype. FIG. 15B highlights the difference in Formula (IIa) in samples taken from patients with HCV genotype 3. FIG. 15C shows the IC₅₀ of Formula (IIa) in samples from patients previously treated with interferon contrasted with the IC₅₀ of Formula (IIa) in samples from patients previously treated with another direct-acting antiviral (DAA), e.g., Formula (IIa).

FIG. 16 is a table summarizing the HCV genotype, patient history, and IC₅₀ determination of Formula (IIa) in serum samples taken from 30 patients infected with various resistant strains of HCV.

FIG. 17 is a table summarizing the results of a study in which the NS5A mutations were determined in samples taken from seven patients infected with resistant strains of HCV. The IC₅₀ values of Formula (IIa) and interferon were also determined in these samples using the capture fusion assay outlined in Example 1.

FIGS. 18A-E depict graphs summarizing the IC₅₀ determination of Formula (IIa) in samples drawn from the seven patients infected with resistant strains of HCV outlined in FIG. 17.

FIGS. 19A-E depict graphs summarizing the IC₅₀ determination of IFN-2a (i.e. peg-interferon 2a) in samples drawn from the seven patients infected with resistant strains of HCV outlined in FIG. 17.

FIG. 20 is a table summarizing the sequencing data for a sample drawn from a patient prior to treatment with Formula (IIa).

FIG. 21 is a table summarizing the sequencing data for a sample drawn from a patient four weeks after failure of EAP treatment.

FIG. 22 is a table summarizing the results of a study in which the IC₅₀ value of Formula (IIa) was measured in a panel of serum samples taken from HCV-infected patients that had not previously responded to current anti-HCV treatment regimes. Analysis of the serum samples also determined the nature of the NS5A mutation in the HCV infection if present.

FIG. 23 is a graph summarizing the IC₅₀ determination of Formula (IIa) in a sample drawn from patient 7 bearing an NS5A L31M mutation as outlined in the table of FIG. 22.

FIGS. 24A-C depict graphs summarizing the response to sofosbuvir in pre-treatment (FIG. 24A) and post-treatment (FIG. 24B) samples taken from a patient who relapsed after treatment with a sofosbuvir-containing anti-HCV therapy and did not possess any known sofosbuvir resistance motifs. The post-treatment sample was sensitive to treatment with Formula (IIa) (FIG. 24C).

### DETAILED DESCRIPTION OF THE INVENTION

The present invention relates to methods of treating a subject infected with the Hepatitis C virus (e.g., a resistant variant of HCV), the method comprising administration of a compound of Formula (I) or a prodrug thereof (e.g., a compound of Formula (II)) or pharmaceutically acceptable salt thereof. The present invention further comprises methods of treating a subject infected with HCV with a compound of Formula (I) or a prodrug thereof (e.g., a compound of Formula (II)) in combination with another agent, e.g., sofosbuvir.

### Definitions

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

As used herein, the term “acquire” or “acquiring” as the terms are used herein, refer to obtaining possession of a physical entity (e.g., a sample, e.g., blood sample or liver biopsy specimen), or a value, e.g., a numerical value, by “directly acquiring” or “indirectly acquiring” the physical entity or value. “Directly acquiring” means performing a process (e.g., an analytical method) to obtain the physical entity or value. “Indirectly acquiring” refers to receiving the physical entity or value from another party or source (e.g., a third party laboratory that directly acquired the physical entity or value). Directly acquiring a value includes performing a process that includes a physical change in a sample or another substance, e.g., performing an analytical process which includes a physical change in a substance, e.g., a sample, performing an analytical method, e.g., a method as described herein, e.g., by sample analysis of bodily fluid, such as blood by, e.g., mass spectroscopy (e.g. LC-MS), or PCR (e.g., RT-PCR).

As used herein, the term “prodrug” refers to a compound which, when metabolized (e.g., in vivo or in vitro), yields an active compound. In some embodiments, the prodrug may be inactive, or possess less activity that the free drug, but may provide advantageous handling, administration, or metabolic properties. Exemplary prodrug moieties of the present invention may be linked to the free drug through the hydroxyl, amino, phosphate, or phosphorothioate backbone of the nucleotide, and may comprise an ester, a carbamate, a carbonyl, a thioester, amide, isocyanate, urea, thiourea, or other physiologically acceptable metabolically labile moiety. In some embodiments, a prodrug is activated through enzymatic hydrolysis.

As used herein, an amount of a compound, conjugate, or substance effective to treat a disorder (e.g., a disorder described herein), “therapeutically effective amount,” “effective amount” or “effective course” refers to an amount of the compound, substance, or composition which is effective, upon single or multiple dose administration(s) to a subject, in treating a subject, or in curing, alleviating, relieving or improving a subject with a disorder (e.g., HCV infection) beyond that expected in the absence of such treatment.

As used herein, the terms “prevent” or “preventing” as used in the context of a disorder or disease, refer to administration of an agent to a subject, e.g., the administration of a compound of the present invention (e.g., a compound of Formula (I) or a compound of Formula (II)) to a subject, such that the onset of at least one symptom of the disorder or disease is delayed as compared to what would be seen in the absence of administration of said agent.

As used herein, the term “resistant” or “resistance” refers to a strain of HCV that is not substantially diminished or inactivated upon administration with an anti-HCV agent. In some embodiments, a resistant HCV strain comprises a protein (e.g., an E1, E2, NS1, NS2, NS3, NS4A, NS4B, NS5A, or NS5B protein) that substantially maintains its activity in the presence of an anti-HCV agent known to inhibit said protein. In some embodiments, a resistant HCV strain comprises a protein bearing an amino acid mutation (e.g., an amino acid substitution, addition, or deletion) compared with a reference sequence of said protein. In some embodiments, an HCV protein bearing an amino acid mutation (e.g., an amino acid substitution, addition, or deletion) may result in aberrant function of said protein or affect the inhibition of said protein with an anti-HCV agent. In some embodiments, the level of resistance may be determined through a measurement of viral load or other biomarker in a sample (e.g., a serum sample), or through the determination of the IC₅₀ value of a specific antiviral agent other agents beyond Formula (I) and Formula (II) in a sample (e.g., a serum sample).

As used herein, the term “subject” is intended to include human and non-human animals. Exemplary human subjects include a human patient having a disorder, e.g., a disorder described herein (e.g., HCV infection), or a normal subject. The term “non-human animals” includes all vertebrates, e.g., non-mammals (such as chickens, amphibians, reptiles) and mammals, such as non-human primates, domesticated and/or agriculturally useful animals, e.g., sheep, dogs, cats, cows, pigs, etc.

As used herein, the terms “treat” or “treating” a subject having a disorder or disease refer to subjecting the subject to a regimen, e.g., the administration of a compound of Formula (I) or a prodrug (e.g., a compound of Formula (II)) or pharmaceutically acceptable salt thereof, or a composition comprising Formula (I) or a prodrug (e.g., a compound of Formula (II)) or pharmaceutically acceptable salt thereof, such that at least one symptom of the disorder or disease is cured, healed, alleviated, relieved, altered, remedied, ameliorated, or improved. Treating includes administering an amount effective to alleviate, relieve, alter, remedy, ameliorate, improve or affect the disorder or disease, or the symptoms of the disorder or disease. The treatment may inhibit deterioration or worsening of a symptom of a disorder or disease.

Numerous ranges, e.g., ranges for the amount of a drug administered per day, are provided herein. In some embodiments, the range includes both endpoints. In other embodiments, the range excludes one or both endpoints. By way of example, the range can exclude the lower endpoint. Thus, in such an embodiment, a range of 100 to 1000 mg/day, excluding the lower endpoint, would cover an amount greater than 100 that is less than or equal to 1000 mg/day.

“Co-administration”, “co-administering,” or “co-providing”, as used herein in the context of the administration of therapies, refers to administration at the same time, administration of one therapy before (e.g., immediately before, less than about 5, about 10, about 15, about 30, about 45, about 60 minutes, about 1, about 2, about 3, about 4, about 6, about 8, about 10, about 12, about 16, about 20, about 24, about 48, about 72 or more hours before) administration of a secondary therapy.

“Course of therapy”, as referred to herein, comprises one or more separate administrations of a therapeutic agent (e.g., a compound of Formula (I) or a prodrug (e.g., a compound of Formula (II)) or pharmaceutically acceptable salt thereof). A course of therapy can comprise one or more cycles of a therapeutic agent.

A “cycle”, as used herein in the context of a cycle of administration of a drug, refers to a period of time for which a drug is administered to a patient. For example, if a drug is administered for a cycle of 4 weeks days, the periodic administration, e.g., daily or twice daily, is given for 4 weeks. A drug can be administered for more than one cycle. In some embodiments, the first and second or subsequent cycles are the same in terms of one or both of duration and periodic administration. In embodiments, a first and second or subsequent cycle differs in terms of one or both of duration and periodic administration. Rest periods may be interposed between cycles. A rest cycle may be about 1, about 2, about 4, about 6, about 8, about 10, about 12, about 16, about 20, or about 24 hours; or about 1, about 2, about 3, about 4, about 5, about 6, or about 7 days; or about 1, about 2, about 3, about 4 or more weeks in length.

### Compounds and Therapeutic Agents

The present invention features methods for treatment of a subject infected with HCV or a resistant variant thereof comprising administration of a composition comprising a compound of Formula (I) or a prodrug or pharmaceutically acceptable salt thereof. The active agent is Formula (I), e.g., any one of Formula (Ia), Formula (Ib), and Formula (Ic), or a combination thereof:

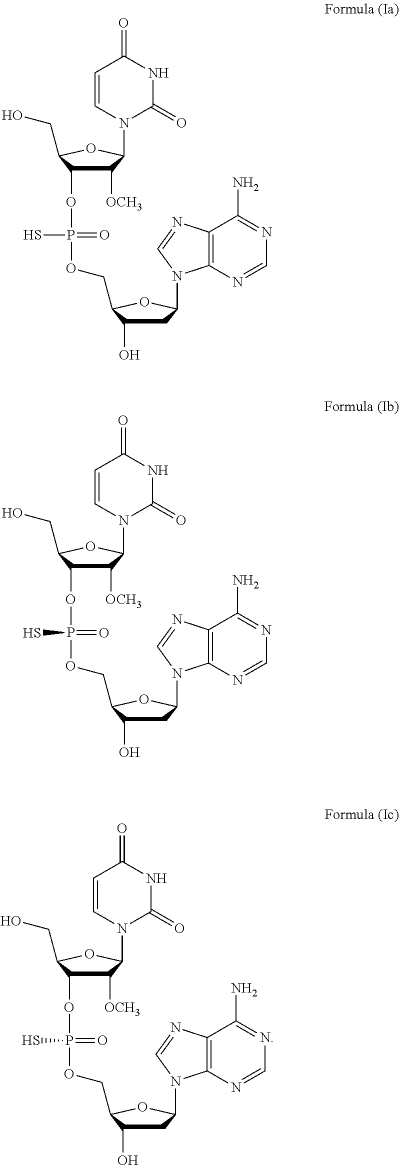

The composition of the present invention may comprise a prodrug of Formula (I), wherein said prodrug is a compound of Formula (II). The prodrug (e.g., the compound of Formula (II)) may be described by any one of Formula (IIa), Formula (IIb), and Formula (IIc), or a combination thereof:

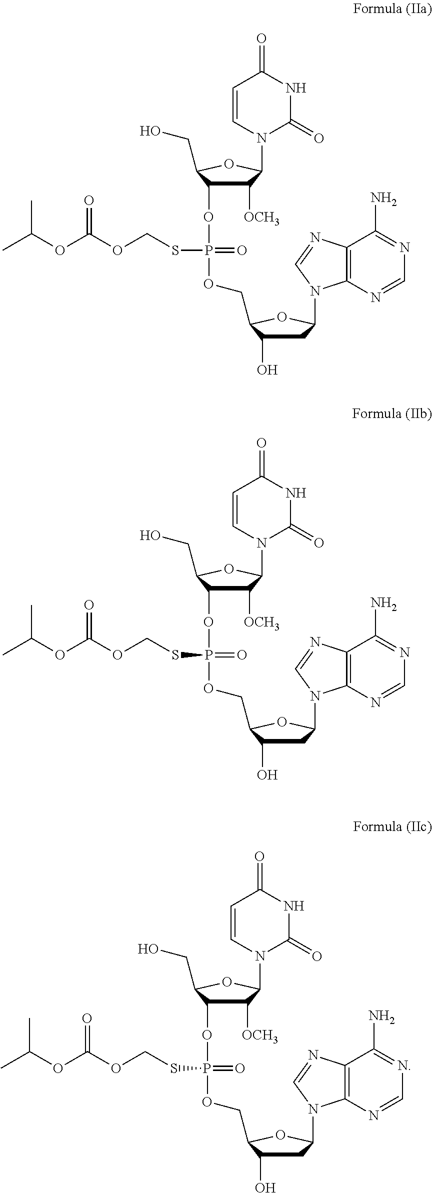

Formula (I) and its prodrug Formula (II) are small molecule nucleic acid hybrid (dinucleotide) compounds that combine both antiviral and immune modulating activities. The latter activity mediates controlled apoptosis of virus-infected hepatocytes via stimulation of the innate immune response, similar to what is also achieved by IFN-α therapy in HCV-infected patients.

Without wishing to be bound by theory, the mechanism of action of Formula (I) and its prodrug Formula (II) may be dissected into two components. The first component entails the host immune stimulating activity of Formula (I), which induces endogenous IFNs via the activation of viral sensor proteins, e.g., retinoic acid-inducible gene 1 (RIG-I) and nucleotide-binding oligomerization domain-containing protein 2 (NOD2) (Takeuchi, O. and Akira S. Cell (2010) 140:805-820; Sabbah, A. et al. Nat Immunol (2009) 10:1073-1080). Activation may occur by binding of Formula (I) to the RIG-I/NOD2 proteins at their nucleotide binding domain. The RIG-I and NOD2 proteins are located in the cytosol of cells, including hepatocytes, and usually recognize signature patterns of foreign nucleic acids such as the pathogen associated molecular pattern (PAMP). Once PAMP within viral RNA or DNA is recognized, RIG-I and NOD2 may become activated and trigger the IFN signaling cascade that then results in IFN and interferon-stimulated gene (ISG) production and induction of an antiviral state in cells.

The second component of the mechanism of action of Formula (I) and its prodrug Formula (II) involves its direct antiviral activity, which inhibits the synthesis of viral nucleic acids by steric blockage of the viral polymerase, protease, or other targets. The block may be achieved by interaction of Formula (I) with RIG-I and NOD2 as described above that then in turn may prevent the polymerase enzyme from engaging with the viral nucleic acid template for replication (i.e, HCV pre-genomic RNA).

The second component of the mechanism of action of Formula (I) and its prodrug Formula (II) involves its direct antiviral activity, which inhibits the synthesis of viral nucleic acids by steric blockage of the viral polymerase. The block may be achieved by interaction Formula (I) with RIG-I and NOD2 as described above that then in turn may prevent the polymerase enzyme from engaging with the viral nucleic acid template for replication (i.e, HCV pre-genomic RNA). The cytotoxic potential of Formula (II) (e.g., Formula (IIa)) has been initially evaluated using a panel of cell lines. Similar to the parental drug, Formula (II) demonstrated an excellent safety profile, with a 50% cytotoxic concentration (CC50) of greater than 1000 μM (Coughlin, J. E. et al. Bioorg Med Chem Lett (2010) 20:1783-1786).

In some embodiments, the method described herein comprises administration of a compound of Formula (I), e.g., Formula (Ia), Formula (Ib), or Formula (Ic), or a pharmaceutically acceptable salt thereof. In other embodiments, the method described herein comprises administration of prodrug of Formula (I) (e.g., a compound of Formula (II), e.g., Formula (IIa), Formula (IIb), or Formula (IIc)) or a pharmaceutically acceptable salt thereof. In other embodiments, the method herein describes administration of a composition comprised of a combination of a compound of Formula (I) (e.g., Formula (Ia), Formula (Ib), or Formula (Ic)) and a compound of Formula (II) (e.g., Formula (Ia), Formula (Ib), or Formula (Ic)) or pharmaceutically acceptable salts thereof. It is well established that the prodrug Formula (I) has been shown to be converted to the active drug Formula (I) (e.g., the Rp- and Sp-Formula (I) isomers) upon administration.

The compounds provided herein may contain one or more asymmetric centers and thus occur as racemates and racemic mixtures, single enantiomers, individual diastereomers and diastereomeric mixtures. All such isomeric forms of these compounds are expressly included within the scope. Unless otherwise indicated when a compound is named or depicted by a structure without specifying the stereochemistry and has one or more chiral centers, it is understood to represent all possible stereoisomers of the compound. The compounds provided herewith may also contain linkages (e.g., carbon-carbon bonds, phosphorus-oxygen bonds, or phosphorus-sulfur bonds) or substituents that can restrict bond rotation, e.g. restriction resulting from the presence of a ring or double bond.

### HCV Infection and Drug Resistance

The present invention relates to methods for treating a subject infected with HCV through administration of Formula (I) or the prodrug Formula (II), or a pharmaceutically acceptable salt thereof. HCV is a small, positive sense single-stranded RNA virus of the family Flaviviridae. The virus is characterized into seven major genotypes (genotypes 1-7), each of which differs by about 30-35% of the nucleotide sites throughout the viral genome. In Europe and the Americas, the most prevalent genotypes of HCV are HCV-1a and HCV-1b. In some embodiments, the methods described herein are used to treat a subject suffering from any known form of HCV infection (e.g., any genotype or serotype of HCV or a combination thereof). In some embodiments, the methods described herein are particularly potent for treatment of a subject infected with genotypes HCV-1a and HCV-1b.

Unlike other variants of hepatitis (e.g., hepatitis A and hepatitis B), there is currently no vaccine available to prevent HCV infection. The goal of current HCV therapies to achieve a sustained viral response (SVR), defined as the absence of serum HCV RNA 3-6 months after the completion of therapy, which equated with a cure. Current therapy includes oral or parenteral administration of a nucleoside or nucleotide analog, or administration of interferons (e.g., IFN-α) and alternate formulations (e.g., pegylated IFN-α). However, use of interferon therapy is limited due to the development of unwanted side effects and variability in treatment response of HCV carriers. Combination therapies comprising one or more nucleoside or nucleoside analogs with or without interferon therapy is also available, but these regimens also may present toxicity issues and elicit resistance. Therefore, one goal of current HCV therapy is to develop new antiviral compounds that can mimic the benefits of IFN therapy but induce suppression of HCV replication and limit the development of resistant strains.

Naturally, HCV exists within a host as a population of genetically distinct but closely related virions (Pawlotsky, J. M. Clin Liv Dis (2003) 7:45-66; Strahotin, C. S. and Babich, M. Adv Virol (2012) ID 267483). Treatment with standard anti-HCV agents may eliminate some or nearly all of the HCV population, leaving behind a small and at times undetectable HCV population that is resistant to said treatment and may develop into a chronic infection. For many years, the standard treatment for chronic HCV has been a combination of pegylated IFN-α and ribavirin; however, neither drug exerts viral pressure and thus can be ineffective for certain patient populations (Strahotin, C. S. and Babich, M. Adv Virol (2012) ID 267483).

Without being bound by any particular theory, a drug-resistant strain of HCV may comprise an amino acid mutation (e.g., an amino acid substitution, addition, or deletion) in a particular protein that may result in a structural change, e.g., a conformational or steric change, that affects the ability of an anti-HCV agent from binding to said protein and modulating its activity, e.g., through inhibiting HCV replication or pathogenicity. Particularly, amino acids in and around the active site or close to the inhibitor binding site may be mutated such that the activity of the protein is impacted. In some instances, the amino acid mutation (e.g., an amino acid substitution, addition, or deletion) may be conservative and may not substantially impact the structure or function of a protein. For example, in certain cases, the substitution of a serine residue with a threonine residue may not significantly impact the function of a protein. In other cases, the amino acid mutation may be more dramatic, such as the substitution of a charged amino acid (e.g., aspartic acid or lysine) with a large, nonpolar amino acid (e.g., phenylalanine or tryptophan) and therefore may have a substantial impact on protein function. The nature of the mutations that render the HCV strain resistant to one or more antiviral agents can be readily identified using standard sequencing techniques, e.g., deep sequencing techniques, that are well known in the art.

In some embodiments, the drug-resistant HCV strain comprises a variant or mutant form of the E1, E2, NS1, NS2, NS3, NS4A, NS4B, NS5A, or NS5B proteins. In some embodiments, the drug-resistant HCV strain comprises a variant or mutant form of the E1, E2, NS1, NS2, NS3, NS4A, NS4B, NS5A, or NS5B proteins compared with the accepted consensus sequence of said proteins. In some embodiments, the drug-resistant HCV strain comprises a form of the NS5A protein with no sequence variations or mutations, compared with the accepted consensus sequence of the protein.

In some embodiments, the drug-resistant HCV variant comprises an amino acid mutation (e.g., an amino acid substitution, addition, or deletion) in the sequence of the E1, E2, NS1, NS2, NS3, NS4A, NS4B, NS5A, or NS5B proteins. In some embodiments, the drug-resistant HCV variant comprises an amino acid mutation (e.g., an amino acid substitution, addition, or deletion) in the sequence of the E1 protein. In some embodiments, the drug-resistant HCV variant comprises an amino acid mutation (e.g., an amino acid substitution, addition, or deletion) in the sequence of the E2 protein. In some embodiments, the drug-resistant HCV variant comprises an amino acid mutation (e.g., an amino acid substitution, addition, or deletion) in the sequence of the NS1 protein. In some embodiments, the drug-resistant HCV variant comprises an amino acid mutation (e.g., an amino acid substitution, addition, or deletion) in the sequence of the NS2 protein. In some embodiments, the drug-resistant HCV variant comprises an amino acid mutation (e.g., an amino acid substitution, addition, or deletion) in the sequence of the NS3 protein. In some embodiments, the amino acid mutation (e.g., an amino acid substitution, addition, or deletion) in the NS3 protein sequence comprises a mutation at amino acid positions 9, 16, 18, 23, 36, 39, 40, 41, 43, 54, 55, 65, 67, 70, 71, 80, 89, 109, 138, 155, 156, 162, 168, 170, 174, 176, 179, 260, or 489. In some embodiments, the drug-resistant HCV variant comprises an amino acid mutation (e.g., an amino acid substitution, addition, or deletion) in the sequence of the NS4A protein. In some embodiments, the drug-resistant HCV variant comprises an amino acid mutation (e.g., an amino acid substitution, addition, or deletion) in the sequence of the NS4B protein. In some embodiments, the amino acid mutation (e.g., an amino acid substitution, addition, or deletion) in the NS5A protein sequence comprises a mutation between amino acids 1 and 447, e.g., as compared to a reference or consensus sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence. In some embodiments, the drug-resistant HCV variant comprises an amino acid mutation (e.g., an amino acid substitution, addition, or deletion) in the sequence of the NS5B protein. In some embodiments, the amino acid mutation (e.g., an amino acid substitution, addition, or deletion) in the NS5B protein sequence comprises a mutation at amino acid positions 15, 95, 96, 142, 152, 156, 222, 223, 244, 282, 309, 310, 320, 321, 326, 329, 333, 365, 411, 414, 415, 423, 445, 448, 451, 452, 495, 554, 558, or 559.

In some embodiments, the drug-resistant HCV variant comprises more than one amino acid mutation (e.g., an amino acid substitution, addition, or deletion) in the sequence of the E1, E2, NS1, NS2, NS3, NS4A, NS4B, NS5A, or NS5B proteins. In some embodiments, the drug-resistant HCV variant comprises at least 2, at least 3, at least 4, at least 5, at least 6, at least 7, at least 8, at least 9, at least 10, at least 12, at least 15, at least 20, at least 25, at least 30, at least 35, at least 40, at least 45, at least 50 or more amino acid mutations (e.g., an amino acid substitution, addition, or deletion) in the sequence of the E1, E2, NS1, NS2, NS3, NS4A, NS4B, NS5A, or NS5B proteins. In some embodiments, the drug-resistant HCV variant comprises an amino acid mutation (e.g., an amino acid substitution, addition, or deletion) in the sequence of the only one of the E1, E2, NS1, NS2, NS3, NS4A, NS4B, NS5A, or NS5B proteins. In some embodiments, the drug-resistant HCV variant comprises an amino acid mutation (e.g., an amino acid substitution, addition, or deletion) in the sequence of at least 2, at least 3, at least 4, at least 5, at least 6, at least 7, or all of the E1, E2, NS1, NS2, NS3, NS4A, NS4B, NS5A, or NS5B proteins. In some embodiments, the drug-resistant HCV variant may comprise an amino acid mutation in a protein other than the E1, E2, NS1, NS2, NS3, NS4A, NS4B, NS5A, or NS5B proteins. In some embodiments, the protein other than the E1, E2, NS1, NS2, NS3, NS4A, NS4B, NS5A, or NS5B proteins is not the NS5A protein.

In the above embodiments, the amino acid mutation (e.g., an amino acid substitution, addition, or deletion) in the drug-resistant HCV strain comprises a variant or mutant form of the E1, E2, NS1, NS2, NS3, NS4A, NS4B, NS5A, or NS5B proteins compared with the accepted consensus sequence or a reference sequence of said proteins.

In some embodiments, the drug-resistant HCV strain comprises an amino acid mutation (e.g., an amino acid substitution, addition, or deletion) in the NS5A protein and may further comprise an amino acid mutation (e.g., an amino acid substitution, addition, or deletion) in a protein other than the NS5A protein, e.g., the E1, E2, NS1, NS2, NS3, NS4A, NS4B, or NS5B proteins, e.g., as compared to a reference or consensus sequence. In some embodiments, the drug-resistant HCV strain may further comprise an amino acid mutation (e.g., an amino acid substitution, addition, or deletion) in the E1 protein, e.g., as compared to a reference or consensus sequence. In some embodiments, the drug-resistant HCV strain may further comprise an amino acid mutation (e.g., an amino acid substitution, addition, or deletion) in the E2 protein, e.g., as compared to a reference or consensus sequence. In some embodiments, the drug-resistant HCV strain may further comprise an amino acid mutation (e.g., an amino acid substitution, addition, or deletion) in the NS1 protein, e.g., as compared to a reference or consensus sequence. In some embodiments, the drug-resistant HCV strain may further comprise an amino acid mutation (e.g., an amino acid substitution, addition, or deletion) in the NS2 protein, e.g., as compared to a reference or consensus sequence. In some embodiments, the drug-resistant HCV strain may further comprise an amino acid mutation (e.g., an amino acid substitution, addition, or deletion) in the NS3 protein, e.g., as compared to a reference or consensus sequence. In some embodiments, the drug-resistant HCV strain may further comprise an amino acid mutation (e.g., an amino acid substitution, addition, or deletion) in the NS4A protein, e.g., as compared to a reference or consensus sequence. In some embodiments, the drug-resistant HCV strain may further comprise an amino acid mutation (e.g., an amino acid substitution, addition, or deletion) in the NS4B protein, e.g., as compared to a reference or consensus sequence. In some embodiments, the drug-resistant HCV strain may further comprise an amino acid mutation (e.g., an amino acid substitution, addition, or deletion) in the NS5B protein, e.g., as compared to a reference or consensus sequence.

In some embodiments, the drug-resistant HCV strain comprising a variant or mutant form of the NS5A protein may further comprise more than one amino acid mutation (e.g., an amino acid substitution, addition, or deletion) in the sequence of the E1, E2, NS1, NS2, NS3, NS4A, NS4B, or NS5B proteins. In some embodiments, the drug-resistant HCV strain comprising a variant or mutant form of the NS5A protein may further comprise at least 2, at least 3, at least 4, at least 5, at least 6, at least 7, at least 8, at least 9, at least 10, at least 12, at least 15, at least 20, at least 25, at least 30, at least 35, at least 40, at least 45, at least 50 or more amino acid mutations (e.g., an amino acid substitution, addition, or deletion) in the sequence of the E1, E2, NS1, NS2, NS3, NS4A, NS4B, or NS5B proteins. In some embodiments, the drug-resistant HCV strain comprises an amino acid mutation (e.g., an amino acid substitution, addition, or deletion) in the sequence of the only one of the E1, E2, NS1, NS2, NS3, NS4A, NS4B, or NS5B proteins. In some embodiments, the drug-resistant HCV strain comprises an amino acid mutation (e.g., an amino acid substitution, addition, or deletion) in the sequence of at least 2, at least 3, at least 4, at least 5, at least 6, at least 7, or all of the E1, E2, NS1, NS2, NS3, NS4A, NS4B, or NS5B proteins. In some embodiments, the drug-resistant HCV strain may comprise an amino acid mutation in a protein other than the E1, E2, NS1, NS2, NS3, NS4A, NS4B, or NS5B proteins. In the above embodiments, the amino acid mutation (e.g., an amino acid substitution, addition, or deletion) in the drug-resistant HCV strain comprises a variant or mutant form of the E1, E2, NS1, NS2, NS3, NS4A, NS4B, or NS5B proteins compared with the accepted consensus sequence or a reference sequence of said proteins.

In some embodiments, the drug-resistant HCV variant comprises an amino acid mutation (e.g., an amino acid substitution, addition, or deletion) in the sequence of the NS5A protein, e.g., as compared to a reference or consensus sequence. In some embodiments, the amino acid mutation in the sequence of the NS5A protein is an amino acid substitution. In some embodiments, the amino acid mutation in the sequence of the NS5A protein is an amino acid addition. In some embodiments, the amino acid mutation in the sequence of the NS5A protein is an amino acid deletion.

In some embodiments, the amino acid mutation (e.g., an amino acid substitution, addition, or deletion) in the NS5A protein sequence comprises a mutation between amino acids 1 and 447, e.g., as compared to a reference or consensus sequence. In some embodiments, the amino acid mutation (e.g., an amino acid substitution, addition, or deletion) in the NS5A protein sequence comprises a mutation at amino acid positions 1, 8, 23, 24, 25, 26, 28, 30, 31, 32, 34, 36, 37, 44, 46, 48, 54, 58, 63, 64, 78, 85, 90, 93, 99, 107, 114, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 318, 320, 326, 346, 347, 348, 349, 356, 367, 368, 370, 388, 390, 392, 393, 395, 397, 399, 400, 401, 403, 404, 405, 410, 413, 439, 441, or 442, e.g., as compared to a reference or consensus sequence.

In some embodiments, the amino acid mutation (e.g., an amino acid substitution, addition, or deletion) in the NS5A protein sequence comprises a mutation at amino acid positions 23, 24, 28, 30, 31, 32, 37, 54, 58, 63, 93, 295, 318, 320, 356, 404, or 442, e.g., as compared to a reference or consensus sequence. In some embodiments, the amino acid mutation (e.g., an amino acid substitution, addition, or deletion) in the NS5A protein sequence comprises a mutation at amino acid positions 1, 8, 26, 30, 31, 32, 34, 37, 44, 46, 48, 58, 64, 78, 85, 90, 99, 107, 121, 123, 131, 135, 144, 158, 161, 171, 174, 176, 181, 183, 197, 199, 213, 215, 226, 240, 241, 245, 248, 280, 285, 288, 293, 295, 296, 298, 299, 305, 308, 310, 311, 315, 326, 346, 347, 348, 349, 367, 368, 370, 388, 390, 392, 393, 395, 397, 399, 400, 401, 403, 404, 405, 410, 413, 439, 441, or 442, e.g., as compared to a reference or consensus sequence.

In some embodiments, the amino acid mutation in the NS5A protein sequence comprises an amino acid substitution of the wild type amino acid residue present at a particular position in the sequence with another amino acid selected from one of the naturally occurring amino acids. In some embodiments, the amino acid mutation in the NS5A protein sequence comprises an amino acid substitution of the wild type amino acid residue present at a particular position in the sequence with an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

In some embodiments, the amino acid mutation in the NS5A protein sequence comprises an amino acid addition to the wild type sequence at a particular position of an amino acid selected from one of the naturally occurring amino acids. In some embodiments, the amino acid mutation in the NS5A protein sequence comprises an amino acid addition to the wild type sequence at a particular position selected from an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine residue.

In some embodiments, the amino acid mutation in the NS5A protein sequence comprises an amino acid deletion at a particular position of the wild type sequence. In some embodiments, the amino acid deletion in the NS5A protein sequence comprises an amino acid deletion of an alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, or valine residue.

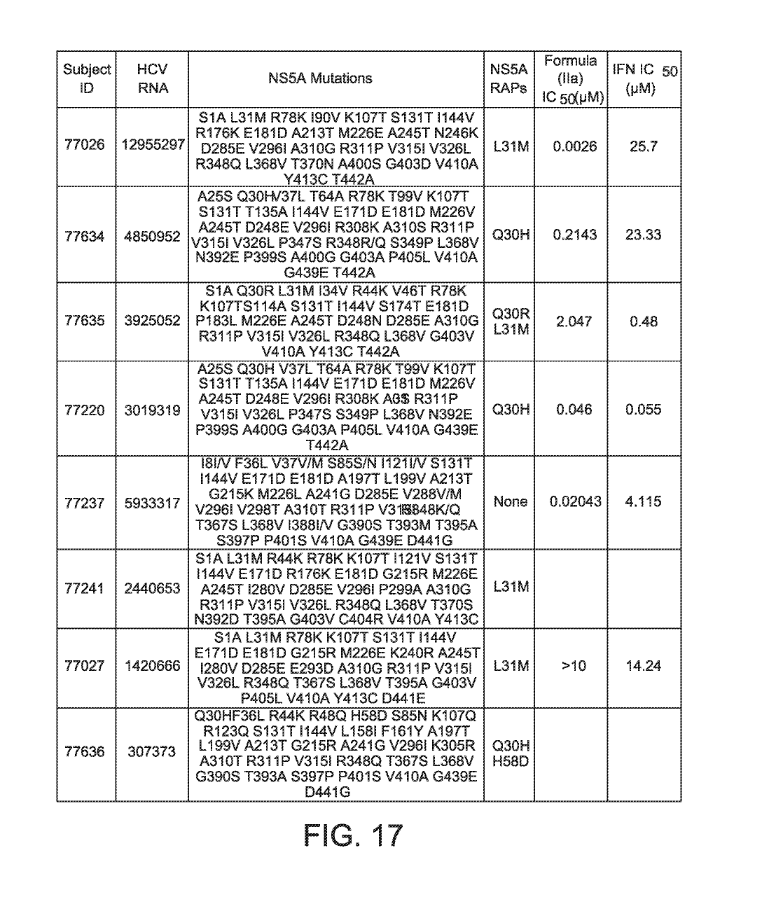

In some embodiments, the amino acid mutation (e.g., an amino acid substitution, addition, or deletion) in the NS5A protein sequence comprises a S1A, L31M, R78K, 190V, K107T, S131T, I144V, R176K, E181D, A213T, M226E, A245T, N246K, D285E, V296I, A310G, R311P, V315I, V326L, R348Q, L368V, T370N, A400S, G403D, V410A, Y413C, or T442A mutation, e.g., as depicted in FIG. 17. In some embodiments, the amino acid mutation (e.g., an amino acid substitution, addition, or deletion) in the NS5A protein sequence comprises a A25S, Q30H, V37L, T64A, R78K, T99V, K107T, S131T, T135A, I144V, E171D, E181D, M226V, A245T, D248E, V296I, R308K, A310S, R311P, V315I, V326L, P347S, R348R/Q, S349P, L368V, N392E, P399S, A400G, G403A, P405L, V410A, G439E, or T442A mutation, e.g., as depicted in FIG. 17. In some embodiments, the amino acid mutation (e.g., an amino acid substitution, addition, or deletion) in the NS5A protein sequence comprises a S1A, Q30R, L31M, I34V, R44K, V46T, R78K, K107T, S114A, S131T, I144V, S174T, E181D, P183L, M226E, A245T, D248N, D285E, A310G, R311P, V315I, V326L, R348Q, L368V, G403V, V410A, Y413C, or T442A mutation, e.g., as depicted in FIG. 17. In some embodiments, the amino acid mutation (e.g., an amino acid substitution, addition, or deletion) in the NS5A protein sequence comprises a A25S, Q30H, V37L, T64A, R78K, T99V, K107T, S131T, T135A, I144V, E171D, E181D, M226V, A245T, D248E, V296I, R308K, A310S, R311P, V315I, V326L, P347S, S349P, L368V, N392E, P399S, A400G, G403A, P405L, V410A, G439E, or T442A mutation, e.g., as depicted in FIG. 17. In some embodiments, the amino acid mutation (e.g., an amino acid substitution, addition, or deletion) in the NS5A protein sequence comprises an I8V, F26L, V37M, S85N, I121V, S131T, I144V, E171D, E181D, A197T, L199V, A213T, G215K, M226L, A241G, D248E, V288M, V296I, V298T, A310T, R311P, V315I, R348K, R348Q, T367S, L368V, I388V, G390S, T393M, T395A, S397P, P401S, V410A, G439E, or D441G mutation, e.g., as depicted in FIG. 17. In some embodiments, the amino acid mutation (e.g., an amino acid substitution, addition, or deletion) in the NS5A protein sequence comprises a S1A, L31M, R44K, R78K, K107T, I121V, S131T, I144V, E171D, R176K, E181D, G215R, M226E, A245T, 1280V, D285E, V296I, P299A, A310G, R311P, V315I, V326L, R348Q, L368V, T370S, N392D, T395A, G403V, C404R, V410A, or Y413C mutation, e.g., as depicted in FIG. 17. In some embodiments, the amino acid mutation (e.g., an amino acid substitution, addition, or deletion) in the NS5A protein sequence comprises a S1A, L31M, R78K, K107T, S131T, I144V, E171D, E181D, G215R, M226E, K240R, A245T, 1280V, D285E, E293D, A310G, R311P, V315I, V326L, R348Q, T367S, L368V, T395A, G403V, P405L, V410A, Y413C, or D441E mutation, e.g., as depicted in FIG. 17. In some embodiments, the amino acid mutation (e.g., an amino acid substitution, addition, or deletion) in the NS5A protein sequence comprises a Q30H, F36L, R44K, R48Q, H58D, S85N, K107Q, R123Q, S131T, I144V, L1581, F161Y, A197T, L199V, A213T, G215R, A241G, V296I, K305R, A310T, R311P, V315I, R348Q, T367S, L368V, G390S, T393A, S397P, P401S, V410A, G439E, or D441G mutation, e.g., as depicted in FIG. 17.

In some embodiments, the amino acid mutation (e.g., an amino acid substitution, addition, or deletion) in the NS5A protein sequence comprises a mutation at amino acid position 31 or 93, e.g., as compared to a reference or consensus sequence. In some embodiments, the amino acid mutation (e.g., an amino acid substitution, addition, or deletion) in the NS5A protein sequence comprises a mutation at amino acid positions 31 and 93, e.g., as compared to a reference or consensus sequence. In some embodiments, the amino acid mutation (e.g., an amino acid substitution, addition, or deletion) in the NS5A protein sequence comprises a mutation at amino acid position 31, e.g., as compared to a reference or consensus sequence. In some embodiments, the amino acid mutation (e.g., an amino acid substitution, addition, or deletion) in the NS5A protein sequence comprises a mutation at amino acid position 93, e.g., as compared to a reference or consensus sequence.

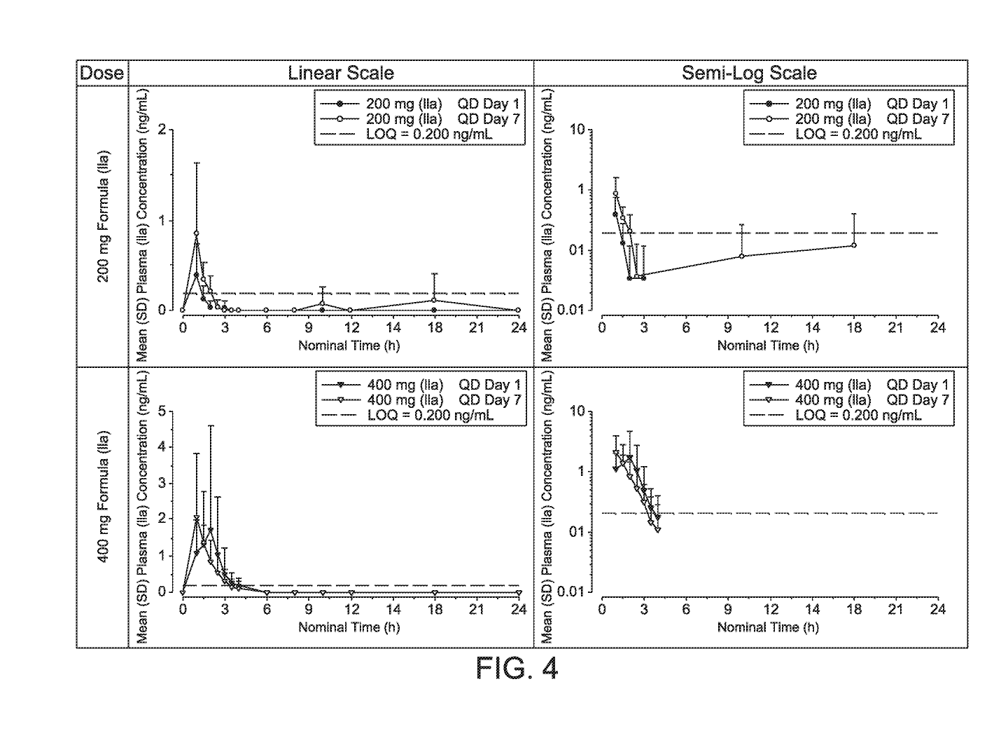

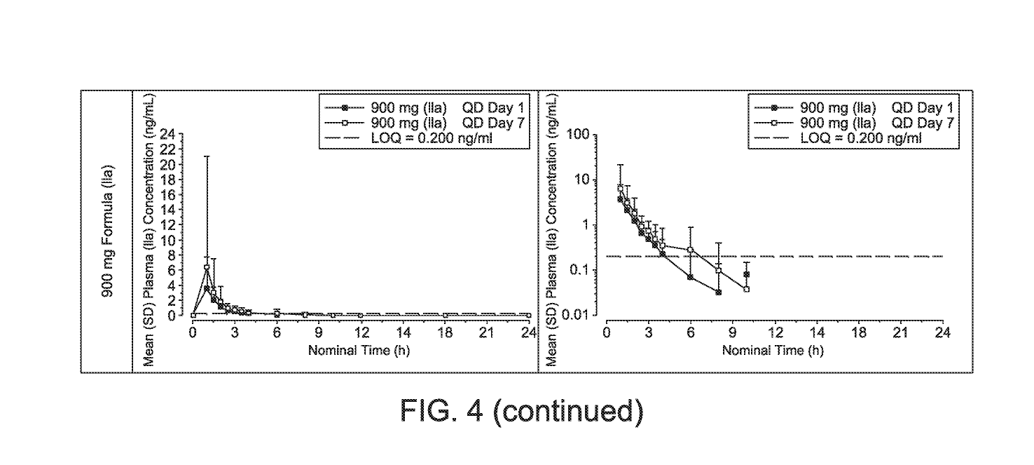

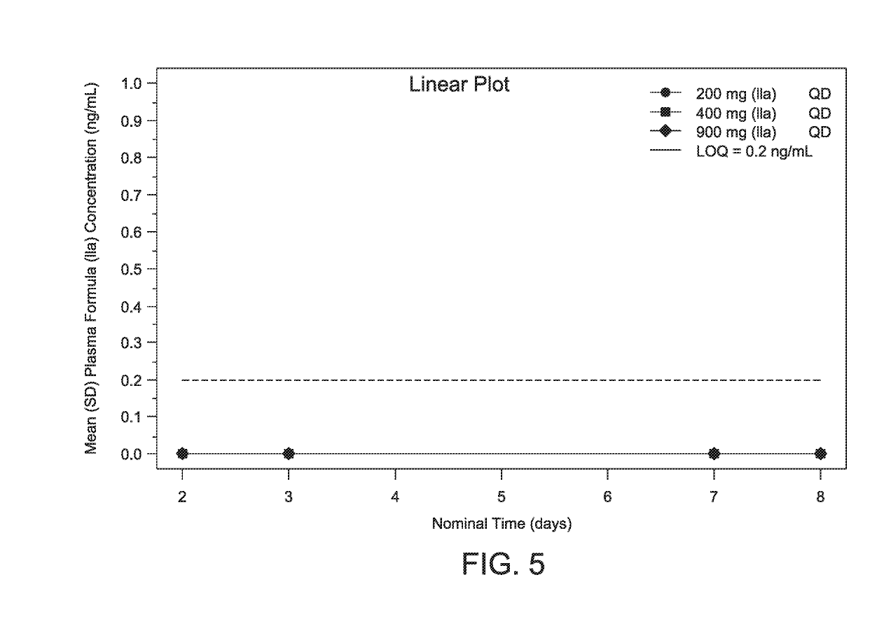

In some embodiments, the amino acid mutation (e.g., an amino acid substitution, addition, or deletion) at position 31 of the NS5A protein sequence comprises an amino acid substitution with an alanine, arginine, asparagine, aspartic acid, cysteine, glutamic acid, glutamine, glycine, histidine, isoleucine, lysine, methionine, phenylalanine, proline, serine, threonine, tryptophan, tyrosine, or valine residue. In some embodiments, the amino acid mutation (e.g., an amino acid substitution, addition, or deletion) at position 31 of the NS5A protein sequence comprises an amino acid substitution with an alanine, arginine, aspartic acid, cysteine, glycine, glutamine, histidine, isoleucine, lysine, methionine, phenylalanine, proline, serine, threonine, tyrosine, or valine residue. In some embodiments, the amino acid mutation (e.g., an amino acid substitution, addition, or deletion) in the NS5A protein sequence comprises a L31A, L31C, L31D, L31F, L31G, L31H, L31I, L31K, L31M, L31P, L31Q, L31R, L31S, L31T, L31V, or L31Y mutation, e.g., as depicted in FIG. 4 or FIG. 5. In some embodiments, the amino acid mutation (e.g., an amino acid substitution, addition, or deletion) in the NS5A protein sequence comprises a L31F, L31H, L31I, L31P, L31R, or L31V mutation, e.g., as depicted in FIG. 4 or FIG. 5. In some embodiments, the amino acid mutation (e.g., an amino acid substitution, addition, or deletion) in the NS5A protein sequence comprises a L31F, L31I, L31M, or L31V mutation, e.g., as depicted in FIG. 4 or FIG. 5. In some embodiments, the amino acid mutation (e.g., an amino acid substitution, addition, or deletion) in the NS5A protein sequence comprises a L31P, L31R, or L31V mutation, e.g., as depicted in FIG. 4 or FIG. 5.

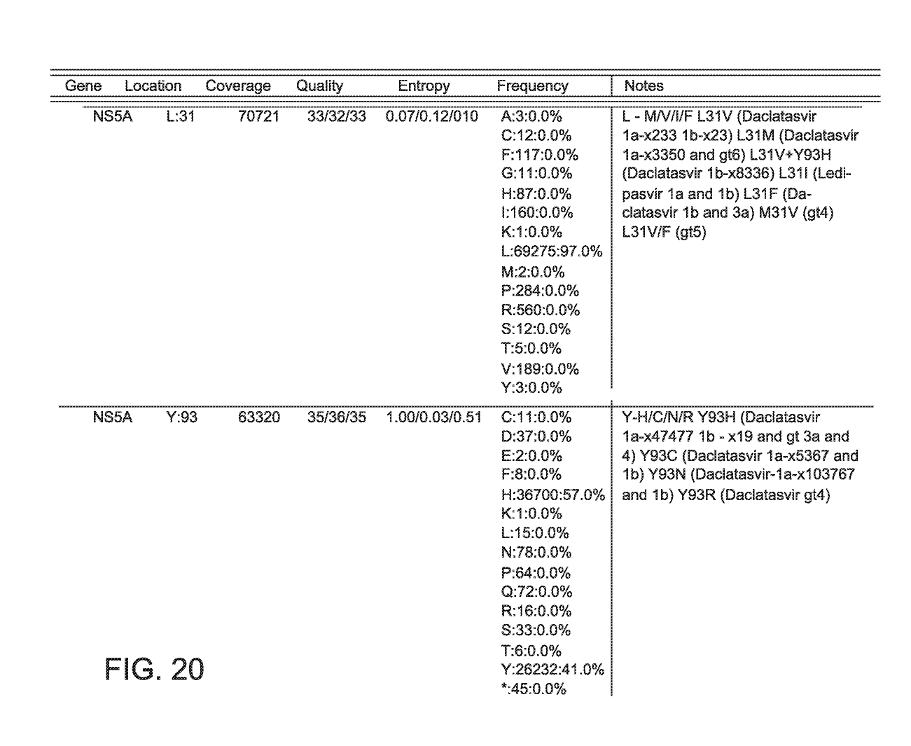

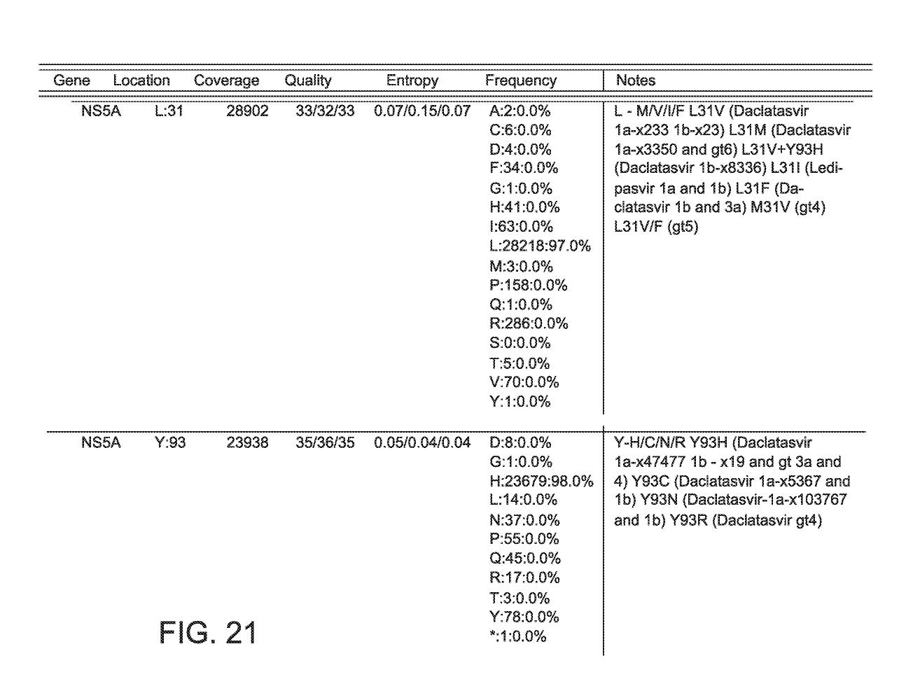

In some embodiments, the amino acid mutation (e.g., an amino acid substitution, addition, or deletion) at position 93 of the NS5A protein sequence comprises an amino acid substitution with an alanine, arginine, asparagine, aspartic acid, cysteine, glutamic acid, glutamine, glycine, histidine, isoleucine, leucine, lysine, methionine, phenylalanine, proline, serine, threonine, tryptophan, or valine residue. In some embodiments, the amino acid mutation (e.g., an amino acid substitution, addition, or deletion) at position 93 of the NS5A protein sequence comprises an amino acid substitution with an arginine, asparagine, aspartic acid, cysteine, glycine, glutamic acid, glutamine, histidine, leucine, lysine, phenylalanine, proline, serine, or threonine residue. In some embodiments, the amino acid mutation (e.g., an amino acid substitution, addition, or deletion) in the NS5A protein sequence comprises a Y93C, Y93D, Y93E, Y93F, Y93G, Y93H, Y93K, Y93L, Y93N, Y93P, Y93Q, Y93R, Y93S, or Y93T mutation, e.g., as depicted in FIG. 20 or FIG. 21. In some embodiments, the amino acid mutation (e.g., an amino acid substitution, addition, or deletion) in the NS5A protein sequence comprises a Y93D, Y93H, Y93N, Y93P, Y93Q, or Y93S mutation, e.g., as depicted in FIG. 20 or FIG. 21. In some embodiments, the amino acid mutation (e.g., an amino acid substitution, addition, or deletion) in the NS5A protein sequence comprises a Y93C, Y93H, Y93N, or Y93R mutation, e.g., as depicted in FIG. 20 or FIG. 21. In some embodiments, the amino acid mutation (e.g., an amino acid substitution, addition, or deletion) in the NS5A protein sequence comprises a Y93H mutation, e.g., as depicted in FIG. 20 or FIG. 21.

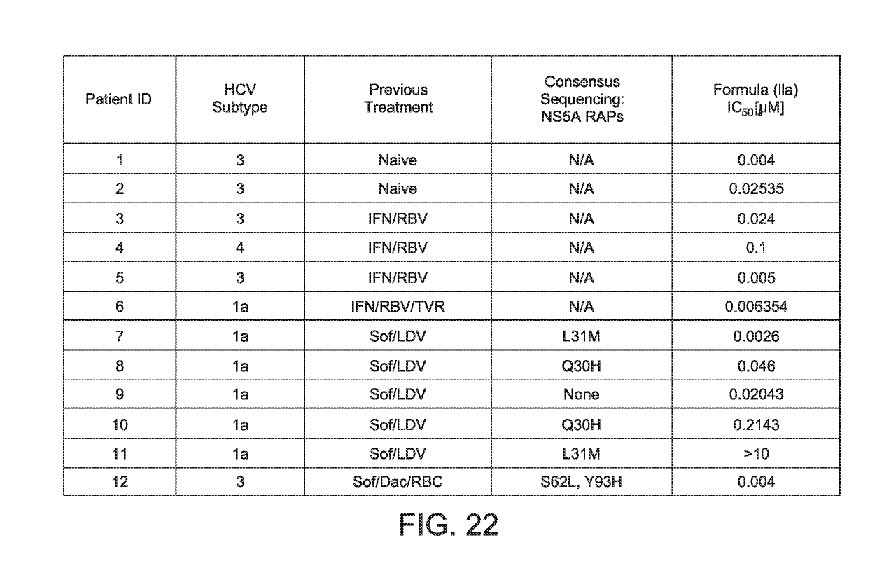

In some embodiments, the amino acid mutation (e.g., an amino acid substitution, addition, or deletion) in the NS5A protein sequence comprises a mutation at amino acid position 30 or 62, e.g., as compared to a reference or consensus sequence. In some embodiments, the amino acid mutation (e.g., an amino acid substitution, addition, or deletion) at position 30 of the NS5A protein sequence comprises an amino acid substitution with an alanine, arginine, asparagine, aspartic acid, cysteine, glutamic acid, glycine, histidine, isoleucine, leucine, lysine, methionine, phenylalanine, proline, serine, threonine, tryptophan, tyrosine, or valine residue. In some embodiments, the amino acid mutation (e.g., an amino acid substitution, addition, or deletion) at position 30 of the NS5A protein sequence comprises an amino acid substitution with a histidine residue. In some embodiments, the amino acid mutation (e.g., an amino acid substitution, addition, or deletion) in the NS5A protein sequence comprises a Q30H mutation, e.g., as depicted in FIG. 22.

In some embodiments, the amino acid mutation (e.g., an amino acid substitution, addition, or deletion) at position 62 of the NS5A protein sequence comprises an amino acid substitution with an alanine, arginine, asparagine, aspartic acid, cysteine, glutamic acid, glutamine, glycine, histidine, isoleucine, leucine, lysine, methionine, phenylalanine, proline, threonine, tryptophan, tyrosine, or valine residue. In some embodiments, the amino acid mutation (e.g., an amino acid substitution, addition, or deletion) at position 30 of the NS5A protein sequence comprises an amino acid substitution with a leucine residue. In some embodiments, the amino acid mutation (e.g., an amino acid substitution, addition, or deletion) in the NS5A protein sequence comprises a S62L mutation, e.g., as depicted in FIG. 22.

In some embodiments, the drug-resistant HCV strain comprises more than one amino acid mutation (e.g., an amino acid substitution, addition, or deletion) to the sequence of the NS5A protein, e.g., as compared to a reference or consensus sequence. In some embodiments, the drug-resistant HCV strain comprises at least 2, at least 3, at least 4, at least 5, at least 6, at least 7, at least 8, at least 9, at least 10, at least 12, at least 15, at least 20, at least 25, at least 30, at least 35, at least 40, at least 45, at least 50 or more amino acid mutations (e.g., an amino acid substitution, addition, or deletion) to the sequence of the NS5A protein, e.g., as compared to a reference or consensus sequence, e.g., as described above.

In some embodiments, the drug-resistant variant of HCV is resistant to an anti-HCV agent other than a compound other than Formula (I) or Formula (II) or a pharmaceutically acceptable salt thereof. In some embodiments, the drug-resistant variant of HCV is resistant to an interferon, a nucleoside analog, a non-nucleoside antiviral, a non-interferon immune enhancer, or a direct-acting antiviral, each of which does not include a compound of Formula (I) or Formula (II) or a pharmaceutically acceptable salt thereof. In some embodiments, the drug-resistant variant of HCV is resistant to sofosbuvir, interferon (e.g., peg-interferon), ribavirin, telaprevir, ledipasvir, danoprevir, ombitasvir, daclatsavir, dasabuvir, boceprevir, ciluprevir, simeprevir, paritaprevir, asunaprevir, tegobuvir, GS-9256, or a combination thereof. In some embodiments, the drug-resistant variant of HCV is resistant to an interferon (e.g., peg-interferon). In some embodiments, the drug-resistant variant of HCV is resistant to ribavirin. In some embodiments, the drug-resistant variant of HCV is resistant to an interferon (e.g., peg-interferon) and ribavirin. In some embodiments, the drug-resistant variant of HCV is resistant to sofosbuvir, telaprevir, ledipasvir, danoprevir, or daclatsavir. In some embodiments, the drug-resistant HCV variant is resistant to more than one anti-HCV agent.

In some embodiments, the IC₅₀ of an anti-HCV agent other than a compound of Formula (I) or Formula (II) in a sample infected with a drug-resistant variant of HCV is higher than the IC₅₀ of a compound of Formula (I) or Formula (II) or a pharmaceutically acceptable salt thereof. In some embodiments, the IC₅₀ of an anti-HCV agent other than a compound of Formula (I) or Formula (II) is more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 95% higher than the IC₅₀ of a compound of Formula (I) or Formula (II) or a pharmaceutically acceptable salt thereof. In some embodiments, the IC₅₀ of an anti-HCV agent other than a compound of Formula (I) or Formula (II) is more than about 1.5 fold, about 2 fold, about 2.5 fold, about 3 fold, about 3.5 fold, about 4 fold, about 4.5 fold, about 5 fold, about 10 fold, about 15 fold, about 20 fold, about 25 fold, about 35 fold, or about 50 fold higher than the IC₅₀ of a compound of Formula (I) or Formula (II) or a pharmaceutically acceptable salt thereof.

### Additional Agents

The present invention features methods for treating a subject infected with HCV (e.g., a resistant variant thereof) through administration of a compound of Formula (I) or Formula (II) or a pharmaceutically acceptable salt thereof. In some embodiments of the present invention, the subject is further administered an additional agent or treatment in conjunction with a compound of Formula (I) or Formula (II). In some embodiments, the additional agent may be an agent for treating an HCV infection. In some embodiments, the additional agent is an interferon, a nucleoside analog, a non-nucleoside antiviral, a non-interferon immune enhancer, or a direct-acting antiviral. In some embodiments, the additional agent is an interferon, e.g., peg-interferon alfa (e.g., peg-interferon alfa-2a or peg-interferon alfa-2b). In some embodiments, the additional agent is a nucleoside or nucleotide analog, e.g., ribavirin or a 2′-C-methyl nucleoside analog. In some embodiments, the additional agent is ribavirin. In some embodiments, the additional agent is a viral protease inhibitor. In some embodiments, the additional agent is an inhibitor of the NS3/4A protease, e.g., telaprevir, ciluprevir, boceprevir, paritaprevir, simeprevir or asunaprevir. In some embodiments, the additional agent is a NS5A inhibitor, e.g., ledipasvir, ombitasvir, dasabuvir, or daclatsavir. In some embodiments, the additional agent is a NS5B inhibitor, e.g., sofosbuvir.

In some embodiments, the combination of a compound of Formula (I) or Formula (II) and the additional agent has a synergistic or additive effect. In some embodiments, the term “additive” refers to an outcome wherein when two agents are used in combination, the combination of the agents acts in a manner equal to but not greater than the sum of the individual anti-HCV activities of each agent.

In some embodiments, the terms “synergy” or “synergistic” refer to an outcome wherein when two agents are used in combination, the combination of the agents acts so as to require a lower concentration of each individual agent than the concentration required to be efficacious in the absence of the other agent. In some embodiments, a synergistic effect results in a reduced in a reduced minimum inhibitory concentration of one or both agents, such that the effect is greater than the sum of the effects. A synergistic effect is greater than an additive effect. In some embodiments, the agents in the composition herein may exhibit a synergistic effect, wherein the anti-HCV activity at a particular concentration is greater than at least about 1.25, 1.5, 1.75, 2, 2.5, 3, 4, 5, 10, 12, 15, 20, 25, 50, or 100 times the anti-HCV activity of either agent alone.

In some embodiments, the additional agent is sofosbuvir. Sofosbuvir, or (2R)-isopropyl 2-(((((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)-phosphoryl)amino)propanoate, is a nucleotide analog prodrug that targets the NS5B viral RNA polymerase. The structure of sofosbuvir is exemplified by Formula (III):

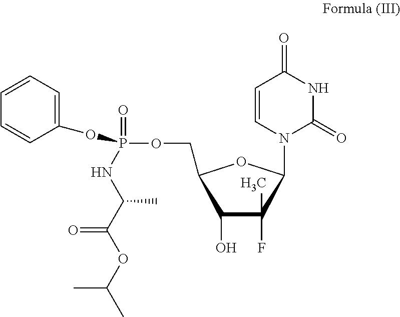

Upon administration, sofosbuvir is metabolized by cellular enzymes to generate the nucleotide monophosphate 2′-deoxy-2′-α-fluoro-β-C-methyluridine-5′-monophosphate. This monophosphate compound is rapidly phosphorylated by cellular kinases to yield the active triphosphate, which is a potent inhibitor of viral RNA synthesis.

Sofosbuvir was first marketed for the treatment of chronic HCV infection in 2013, and has since been approved for the treatment of patients infected with HCV and HIV-1. Sofosbuvir has been shown effective in treatment-naïve patients, as well as for treatment of patients that have previously received anti-HCV therapy. Additionally, patients with compensated cirrhosis and hepatocellular carcinoma, including those awaiting liver transplantation, have been successfully treated with sofosbuvir. In some embodiments, the combination of a compound of Formula (II) and sofosbuvir has a synergistic or additive effect.

### Pharmaceutical Compositions

The present invention features methods for treating a subject infected with HCV, the methods comprising administering a compound of Formula (I) or a prodrug thereof (e.g., a compound of Formula (II)), or a pharmaceutically acceptable salt thereof.

While it is possible for the compound of the present invention (e.g., a compound of Formula (I) or a prodrug thereof (e.g., a compound of Formula (II)) to be administered alone, it is preferable to administer said compound as a pharmaceutical composition or formulation, where the compounds are combined with one or more pharmaceutically acceptable diluents, excipients or carriers. The compounds according to the invention may be formulated for administration in any convenient way for use in human or veterinary medicine. In certain embodiments, the compounds included in the pharmaceutical preparation may be active itself, or may be a prodrug, e.g., capable of being converted to an active compound in a physiological setting (e.g., a compound of Formula (II)). Regardless of the route of administration selected, the compounds of the present invention, which may be used in a suitable hydrated form, and/or the pharmaceutical compositions of the present invention, are formulated into a pharmaceutically acceptable dosage form such as described below or by other conventional methods known to those of skill in the art.

The amount and concentration of compounds of the present invention (e.g., a compound of Formula (I) or a prodrug thereof (e.g., a compound of Formula (II)) in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

Thus, another aspect of the present invention provides pharmaceutically acceptable compositions comprising a therapeutically effective amount or prophylacticaly effective amount of a compound described herein (e.g., a compound of Formula (I) or a prodrug thereof (e.g., a compound of Formula (II)), formulated together with one or more pharmaceutically acceptable carriers (additives) and/or diluents. As described in detail below, the pharmaceutical compositions of the present invention may be specially formulated for administration in solid or liquid form, including those adapted for oral or parenteral administration, for example, by oral dosage, or by subcutaneous, intramuscular or intravenous injection as, for example, a sterile solution or suspension. However, in certain embodiments the subject compounds may be simply dissolved or suspended in sterile water. In certain embodiments, the pharmaceutical preparation is non-pyrogenic, i.e., does not elevate the body temperature of a patient.

The phrases “systemic administration,” “administered systemically,” “peripheral administration” and “administered peripherally” as used herein mean the administration of the compound other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, stabilizing agent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include, but are not limited to: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) ascorbic acid; (17) pyrogen-free water; (18) isotonic saline; (19) Ringer's solution; (20) ethyl alcohol; (21) phosphate buffer solutions; (22) cyclodextrins such as Captisol®; and (23) other non-toxic compatible substances such as antioxidants and antimicrobial agents employed in pharmaceutical formulations.

As set out above, certain embodiments of the compounds described herein may contain a basic functional group, such as an amine, and are thus capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like (see, for example, Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of the compound of the present invention (e.g., a compound of Formula (I) or a prodrug thereof (e.g., a compound of Formula (II) These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like (see, for example, Berge et al., supra).

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The pharmaceutically acceptable carriers, as well as wetting agents, emulsifiers, lubricants, coloring agents, release agents, coating agents, sweetening, flavoring agents, perfuming agents, preservatives, antioxidants, and other additional components may be present in an amount between about 0.001% and 99% of the composition described herein. For example, said pharmaceutically acceptable carriers, as well as wetting agents, emulsifiers, lubricants, coloring agents, release agents, coating agents, sweetening, flavoring agents, perfuming agents, preservatives, antioxidants, and other additional components may be present from about 0.005%, about 0.01%, about 0.05%, about 0.1%, about 0.25%, about 0.5%, about 0.75%, about 1%, about 1.5%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 85%, about 90%, about 95%, or about 99% of the composition described herein.

Pharmaceutical compositions of the present invention may be in a form suitable for oral administration, e.g., a liquid or solid oral dosage form. In some embodiments, the liquid dosage form comprises a suspension, a solution, a linctus, an emulsion, a drink, an elixir, or a syrup. In some embodiments, the solid dosage form comprises a capsule, tablet, powder, dragée, or powder. The pharmaceutical composition may be in unit dosage forms suitable for single administration of precise dosages. Pharmaceutical compositions may comprise, in addition to the compound described herein (e.g., a compound of Formula (I) or a prodrug thereof (e.g., a compound of Formula (II)) or a pharmaceutically acceptable salt thereof, a pharmaceutically acceptable carrier, and may optionally further comprise one or more pharmaceutically acceptable excipients, such as, for example, stabilizers (e.g., a binder, e.g., polymer, e.g., a precipitation inhibitor, diluents, binders, and lubricants.

In some embodiments, the composition described herein comprises a liquid dosage form for oral administration, e.g., a solution or suspension. In other embodiments, the composition described herein comprises a solid dosage form for oral administration capable of being directly compressed into a tablet. In addition, said tablet may include other medicinal or pharmaceutical agents, carriers, and or adjuvants. Exemplary pharmaceutical compositions include compressed tablets (e.g., directly compressed tablets), e.g., comprising a compound of the present invention (e.g., a compound of Formula (I) or a prodrug thereof (e.g., a compound of Formula (II)) or a pharmaceutically acceptable salt thereof.

Formulations of the present invention include those suitable for parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about 99 percent of active ingredient, preferably from about 5 percent to about 70 percent, most preferably from about 10 percent to about 30 percent. Pharmaceutical compositions of this invention suitable for parenteral administration comprise compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a compound of the present invention (e.g., a compound of Formula (I), or a prodrug thereof (e.g., a compound of Formula (II)), it may be desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered form of the compound of the present invention is accomplished by dissolving or suspending compound in an oil vehicle.

In some embodiments, it may be advantageous to administer the compound of the present invention (e.g., a compound of Formula (I) or a prodrug thereof (e.g., a compound of Formula (II)) in a sustained fashion. It will be appreciated that any formulation that provides a sustained absorption profile may be used. In certain embodiments, sustained absorption may be achieved by combining a compound of the present invention with other pharmaceutically acceptable ingredients, diluents, or carriers that slow its release properties into systemic circulation.

### Routes of Administration

The compounds and compositions used in the methods described herein may be administered to a subject in a variety of forms depending on the selected route of administration, as will be understood by those skilled in the art. Exemplary routes of administration of the compositions used in the methods described herein include topical, enteral, or parenteral applications. Topical applications include but are not limited to epicutaneous, inhalation, enema, eye drops, ear drops, and applications through mucous membranes in the body. Enteral applications include oral administration, rectal administration, vaginal administration, and gastric feeding tubes. Parenteral administration includes intravenous, intraarterial, intracapsular, intraorbital, intracardiac, intradermal, transtracheal, subcuticular, intraarticular, subcapsular, subarachnoid, intraspinal, epidural, intrastemal, intraperitoneal, subcutaneous, intramuscular, transepithelial, nasal, intrapulmonary, intrathecal, rectal, and topical modes of administration. Parenteral administration may be by continuous infusion over a selected period of time. In exemplary embodiments of the invention, the compositions described herein comprising a compound of Formula (I) or a prodrug thereof (e.g., a compound of Formula (II)) is administered orally. In exemplary embodiments of the invention, the compositions described herein comprising a compound of Formula (I), or a prodrug thereof (e.g., a compound of Formula (II)) is administered intravenously.

In some embodiments, the compositions described herein comprising a compound of Formula (I) or a prodrug thereof (e.g., a compound of Formula (II)) in combination with sofosbuvir is administered orally. In exemplary embodiments of the invention, the compositions described herein comprising a compound of Formula (I) or a prodrug thereof (e.g., a compound of Formula (II)) in combination with sofosbuvir is administered intravenously.

For intravenous, intraperitoneal, or intrathecal delivery or direct injection, the composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

The choice of the route of administration will depend on whether a local or systemic effect is to be achieved. For example, for local effects, the composition can be formulated for topical administration and applied directly where its action is desired. For systemic, long term effects, the composition can be formulated for enteral administration and given via the digestive tract. For systemic, immediate and/or short term effects, the composition can be formulated for parenteral administration and given by routes other than through the digestive tract.

### Dosages

The compositions of the present invention are formulated into acceptable dosage forms by conventional methods known to those of skill in the art. Actual dosage levels of the active ingredients in the compositions of the present invention (e.g., a compound of Formula (I) or a prodrug thereof (e.g., a compound of Formula (II)) may be varied so as to obtain an amount of the active ingredient which is effective to achieve the desired therapeutic response for a particular subject, composition, and mode of administration, without being toxic to the subject. The selected dosage level will depend upon a variety of pharmacokinetic factors including the activity of the particular compositions of the present invention employed, the route of administration, the time of administration, the rate of absorption of the particular agent being employed, the duration of the treatment, other drugs, substances, and/or materials used in combination with the particular compositions employed, the age, sex, weight, condition, general health and prior medical history of the subject being treated, and like factors well known in the medical arts. A physician or veterinarian having ordinary skill in the art can readily determine and prescribe the effective amount of the composition required. For example, the physician or veterinarian can start doses of the substances of the invention employed in the composition at levels lower than that required in order to achieve the desired therapeutic effect and gradually increase the dosage until the desired effect is achieved. In general, a suitable daily dose of a composition of the invention will be that amount of the substance which is the lowest dose effective to produce a therapeutic effect. Such an effective dose will generally depend upon the factors described above. Preferably, the effective daily dose of a therapeutic composition may be administered as two, three, four, five, six or more sub-doses administered separately at appropriate intervals throughout the day, optionally, in unit dosage forms.

Preferred therapeutic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally) to a subject afflicted with the disorders described herein (e.g., HCV infection). Preferred prophylactic dosage levels are between about 0.1 mg/kg to about 1000 mg/kg (e.g., about 0.2 mg/kg, 0.5 mg/kg, 1.0 mg/kg, 1.5 mg/kg, 2 mg/kg, 3 mg/kg, 4 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 80 mg/kg, 90 mg/kg, 100 mg/kg, 125 mg/kg, 150 mg/kg, 175 mg/kg, 200 mg/kg, 250 mg/kg, 300 mg/kg, 350 mg/kg, 400 mg/kg, 450 mg/kg, 500 mg/kg, 600 mg/kg, 700 mg/kg, 800 mg/kg, 900 mg/kg, or 1000 mg/kg) of the composition per day administered (e.g., orally) to a subject. The dose may also be titrated (e.g., the dose may be escalated gradually until signs of toxicity appear, such as headache, diarrhea, or nausea).

The frequency of treatment may also vary. The subject can be treated one or more times per day (e.g., once, twice, three, four or more times) or every so-many hours (e.g., about every 2, 4, 6, 8, 12, or 24 hours). The composition can be administered 1 or 2 times per 24 hours. The time course of treatment may be of varying duration, e.g., for two, three, four, five, six, seven, eight, nine, ten, or more days, two weeks, 1 month, 2 months, 4 months, 6 months, 8 months, 10 months, or more than one year. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

### Patient Selection and Monitoring

The methods of the present invention described herein entail administration of a compound of Formula (I) or a prodrug thereof (e.g., a compound of Formula (II)) or a pharmaceutically acceptable salt thereof for the treatment of HCV infection. Accordingly, a patient and/or subject can be selected for treatment using a compound of Formula (I) or a prodrug thereof (e.g., a compound of Formula (II)) or a pharmaceutically acceptable salt thereof by first evaluating the patient and/or subject to determine whether the subject is infected with HCV and determination of the serotypic and genotypic classification of the virus. A subject can be evaluated as infected with HCV using methods known in the art. The subject can also be monitored, for example, subsequent to administration of a compound described herein (e.g., a compound of Formula (I) or a prodrug thereof (e.g., a compound of Formula (II)) or a pharmaceutically acceptable salt thereof.

In some embodiments, the subject is a mammal. In some embodiments, the subject is a human. In some embodiments, the subject is an adult. In some embodiments, the subject is suffering from an acute form of HCV infection. In some embodiments, the subject is suffering from a chronic form of HCV infection, e.g., chronic hepatitis C (CHC). In some embodiments, the subject has been diagnosed with hepatitis C (e.g., acute or chronic hepatitis C). In some embodiments, the subject is infected with HCV genotype 1 (e.g., HCV-1a, HCV-1b), HCV genotype 2, HCV genotype 3, HCV genotype 4 HCV genotype 5, HCV genotype 6, HCV genotype 7, HCV genotype 8, HCV genotype 9, HCV genotype 10, or HCV genotype 11. In some embodiments, the subject is infected with HCV genotype 1 (e.g., HCV-1a, HCV-1b), HCV genotype 2, HCV genotype 3, HCV genotype 4, HCV genotype 5, or HCV genotype 6. In some embodiments, the subject is infected with HCV genotype 1 (e.g., HCV-1a, HCV-1b). In some embodiments, the subject is infected with HCV genotype 2. In some embodiments, the subject is infected with HCV genotype 3.

In some embodiments, the subject is treatment naïve. In some embodiments, the subject has previously been treated for HCV infection. In some embodiments, the subject is suffering from a relapsed HCV infection. In some embodiments, the subject has been treated with an anti-HCV agent other than a compound of Formula (I) or Formula (II) or a pharmaceutically acceptable salt thereof and is suffering from a relapsed HCV infection. In some embodiments, the subject has been treated with an interferon, a nucleoside analog, a non-nucleoside antiviral, a non-interferon immune enhancer, or a direct-acting antiviral and is suffering from a relapsed HCV infection. In some embodiments, the subject has been treated with an interferon, e.g., peg-interferon alfa (e.g., peg-interferon alfa-2a or peg-interferon alfa-2b) and is suffering from a relapsed HCV infection. In some embodiments, the subject has been treated with ribavirin and is suffering from a relapsed HCV infection. In some embodiments, the subject has been treated with a viral protease inhibitor, e.g., an inhibitor of the NS3/4A protease, e.g., telaprevir, ciluprevir, boceprevir, paritaprevir, simeprevir or asunaprevir, and is suffering from a relapsed HCV infection. In some embodiments, the subject has been treated with a NS5A inhibitor, e.g., ledipasvir, ombitasvir, dasabuvir, or daclatsavir, and is suffering from a relapsed HCV infection. In some embodiments, the subject has been treated with a NS5B inhibitor, e.g., sofosbuvir, and is suffering from a relapsed HCV infection.

In some embodiments, the subject has been diagnosed with cirrhosis of the liver. In some embodiments, the subject has been diagnosed with hepatocellular carcinoma. In some embodiments, the subject has been diagnosed with hepatocellular carcinoma and is awaiting liver transplantation.

In some embodiments, the subject has been further diagnosed with an HIV infection. In some embodiments, the strain of HIV infection is known. In some embodiments, the subject is infected with HIV-1 or HIV-2 (e.g., strain 1 or strain 2).

### Examples

### Example 1. A Phase 1A/1B Multiple Ascending Dose Study to Assess the Safety, Pharmacokinetics and Pharmacodynamics of Formula (IIa) in Treatment Naïve HCV Infected Adults

### Study Objectives

Formula (IIa) is a novel, first-in-class compound that belongs to a new class of pharmaceuticals called small molecule nucleic acid hybrids. The primary objectives of this first-in-human study are to 1) determine the safety and tolerability of single and multiple ascending doses of Formula (IIa) in treatment naïve subjects with chronic HCV infection (CHC), 2) describe the pharmacokinetic (PK) profile of Formula (IIa) and its metabolites the Sp-isomer (e.g., Formula (IIc)) and the Rp-isomer (e.g., Formula (IIb)) over the dose range tested, 3) to explore the PK/pharmacodynamic (PD) relationships across this dose range, 4) determine the impact of food on absorption of Formula (IIa) and 5) select the optimal doses of Formula (IIa) for subsequent clinical trials. In addition, the PK/PD relationship between steady-state exposure parameters of plasma Formula (IIa) and its metabolites the Sp-isomer (e.g., Formula (IIc)) and the Rp-isomer (e.g., Fornula (IIb)) and maximum suppression of viral load are investigated.

### Methods

The study was a Phase 1, two-part, first-in-human study of single (Part A) and multiple (Part B) ascending doses (SAD and MAD) of Formula (IIa) at doses of 100-900 mg given as a monotherapy for up to 7 days. Subjects were enrolled at multiple sites in Australia and New Zealand. For both Parts A and B, the Safety Review Committee (SRC) determined whether dose escalation occurred after review of all safety data and any available PK/PD data. Part A was an open label, randomized SAD study consisting of four cohorts of two subjects each, with one subject randomized to be dosed in the fed state and the other in the fasted state, at doses of 100 mg to 800 mg Formula (IIa). All subjects were given a single dose of Formula (IIa) orally. The dose cohorts for Part A are summarized in Table 1.

TABLE 1
Summary of Subjects in Part A (SAD Study)

| | Cohort 1 | | Cohort 2 | | Cohort 3 | | Cohort 4 | |
|---|---|---|---|---|---|---|---|---|
| | 100 mg | | 200 mg | | 400 mg | | 800 mg | |
| Analysis Set | Fed | Fasted | Fed | Fasted | Fed | Fasted | Fed | Fasted |
| PK (N = 8) | 1 | 1 | 1 | 1 | 1 | 1 | 1 | 1 |

Part B was a blinded, randomized, placebo-controlled MAD study consisting of four cohorts of eight subjects randomized 6:2 to receive Formula (IIa) or placebo. A total of 30 treatment naïve subjects with CHC infection were enrolled in and a total of 29 subjects (22 subjects administered Formula (IIa) and 7 subjects administered placebo) completed Part B of the study. One subject administered placebo withdrew from the study on Day 2. Subjects infected with HCV-1 and HCV-3 genotypes were enrolled, and no subjects infected with HCV-2 participated in the study. To control for the possible effect of the IL28b genotype, the number of subjects with CC alleles was capped at three per cohort.

The starting dose was determined by the SRC based on review of all safety and any available PK/PD data from Part A. All subjects were dosed in the fasted state with Formula (IIa) daily, from Day 1 until Day 8. All subjects had nothing by mouth other than water for at least 10 hours prior to daily dosing and for 4 hours thereafter. Following dose selection by the SRC, the following dose cohorts were retained for Part B, as summarized in Table 2. In Part B, all subjects were administered the study medication orally with approximately 240 mL of water at the same time every day (+/−10 minutes).

TABLE 2
Summary of Subjects in Part B (MAD Study)

| | | | | | Placebo QD | |
|---|---|---|---|---|---|---|
| | | | 900 mg QD | | HCV1 | |
| | 200 mg QD | 400 mg QD | HCV1 | HCV3 | Cohorts | HCV3 |
| Analysis Set | Cohort 1 | Cohort 2 | Cohort 3 | Cohort 4 | 1 to 3 | Cohort 4 |
| PK (N = 22) | 6 | 6 | 6 | 4 | NA | |
| PK/PD ITT (N = 29) | 6 | 6 | 6 | 4 | 6^{a} | 1 |

^{a}Subject withdrew from the study on Day 2.

Blood samples for PK evaluation of Formula (IIa) and its major metabolites (Sp-isomer Formula (IIc) and Rp-isomer Formula (IIb)) in plasma for Parts A and B were collected according to the following sampling schedule (Table 3).

TABLE 3
PK Blood Sampling Schedule for Part A and Part B

| Nominal Time (h) | Part A | Part B (Days 1 and 7) |
|---|---|---|
| 0 | x | x |
| 1 | x | x |
| 1.5 | x | x |
| 2 | x | x |
| 2.5 | x | x |
| 3 | x | x |
| 3.5 | x | x |
| 4 | x | x |
| 6 | x | x |
| 8 | x | x |
| 10 | x | x |
| 12 | x | x |
| 18 | x | x |
| 24 | x | x |
| 36 | x | x^{a} |
| 48 | x | x^{a} |

^{a}This sample was listed in the concentration-time profiles used for the PK analysis, as the blood draw occurred after the second or third daily doses

A subset of blood samples shown in Table 3 above was used for the PK evaluation of plasma trough concentrations (Table 4). In addition, blood sample assessment of antiviral response was collected according to the following sampling schedule for Part B (Table 4).

TABLE 4
Blood Samples for Pharacokinetic C_{trough} and Antiviral Response (Part B)

| | | C_{trough}^{a}/Antiviral Response Day Relative to the First Dose on Day 1 (Day) | | | | | | | | | |
|---|---|---|---|---|---|---|---|---|---|---|---|
| Analyte/Viral Load | Screening | 1 | 1.5^{b} | 2 | 3 | 4 | 5 | 6 | 7 | 14^{c} | 30^{c} |
| (IIa) and its metabolites | | | | x | x | | | | x^{d} | | |
| HCV RNA | x | x | x | x | x | x | x | x | x | x | x |

^{a}Drawn before the next daily dose, except where otherwise indicated.
^{b}Sample taken 12 hours following the first daily dose.
^{c}Follow-up visit.
^{d}The sample collected 24 hours following the Day 7 dose (Table 3) was also considered as a C_{trough} sample.

All plasma PK calculations were performed using actual time points calculated relative to the time of last drug administration. In subjects where at least one Formula (IIa) plasma concentration was measurable, the following PK parameters were determined using noncompartmental (NCA) methods based on individual plasma concentration-time data for Formula (IIa) and its major metabolites (Sp-isomer Formula (IIc) and Rp-isomer Formula (IIb)) in Part A (Day 1) and Part B (Days 1 and 7).

TABLE 5
Pharmacokinetic Paramters of Formula (II), Rp-isomer Formula (Ib),
and Sp-isomer Formula (Ic) (Part A)

| Parameters | Description |
|---|---|
| AUC₀₋ₜ | Area under the plasma concentration time curve from time zero to the time of last |
| | quantifiable concentration (Cₗₐₛₜ), calculated using the linear trapezoid rule. |
| AUC₀₋₂₄ | Area under the plasma concentration time curve from time zero to 24 h, calculated |
| | using the linear trapezoid rule. |
| AUC_{0-∞} | Area under the plasma concentration time curve from time zero to infinity, calculated |
| | using the formula AUG₀₋ₜ + (Cₗₐₛₜ/λ_{z}), where Cₗₐₛₜ is the last quantifiable concentration. |
| | AUC_{0-∞} will be calculated using the linear trapezoid rule. |
| Cₘₐₓ | Maximum observed concentratoin in plasma. |
| Tₘₐₓ | Time to Maximum observed plasma concentration. |
| t_{1/2} | Terminal half-life, calculated as ln(2)/λ_{z}, where λ_{z} = terminal rate constant, computed |
| | by log-linear regression of the terminal log-linear segment of the plasma concentration |
| | versus time curve. |

TABLE 6
Pharmacokinetic Paramters of Formula (II), Rp-isomer Formula (Ib),
and Sp-isomer Formula (Ic) (Part B)

| Parameters | Description |
|---|---|
| AUC₀₋ₜ | Area under the plasma concentration time curve from time zero to the time of last |
| | quantifiable concentration (Cₗₐₛₜ), calculated using the linear trapezoid rule. |
| AUC₀₋₂₄ | Area under the plasma concentration time curve from time zero to 24 h, calculated |
| | using the linear trapezoid rule. This parameter will only be reported on Day 1. |
| AUC_{0-τ} | Area under the plasma concentration time curve from time zero to the end of the dosing |
| | interval τ, where τ is 24 hours, calculated using the linear trapezoid rule (i.e., partial |
| | area from 0-24 h). This parameter will only be reported on Day 7. |
| AUC_{0-∞} | Area under the plasma concentration time curve from time zero to infinity, calculated |
| | using the formula AUG₀₋ₜ + (Cₗₐₛₜ/λ_{z}), where Cₗₐₛₜ is the last quantifiable concentration. |
| | AUC_{0-∞} will be calculated using the linear trapezoid rule. This parameter will only be |
| | reported on Day 1. |
| Cₘₐₓ | Maximum observed concentration in plasma. |
| Tₘₐₓ | Time to Maximum observed plasma concentration. |
| Cₘᵢₙ | Minimum observed concentration in plasma between dose time (0 h) and dose time + τ |
| | on Day 7, where τ is 24 hours. |
| Tₘᵢₙ | Time of minimum observed plasma concentration on Day 7. |
| t_{1/2} | Terminal half-life, calculated as ln(2)/λ_{z}. |
| R(AUC) | Accumulation factor (based on AUC), calculated as AUC_{0-t(Day 7)}/AUC_{0-24(Day 1).} |
| R(Cₘₐₓ) | Accumulation factor (based on Cₘₐₓ), calculated as C_{max(Day 7)}/C_{max(Day 1)} |

### Results

PK analysis was conducted for all enrolled subjects that received any dose of Formula (IIa) with sufficient plasma sample data to assess PK parameters. PK/PD analysis was carried for all enrolled subjects that received any dose of Formula (IIa) and had evaluable PD parameters, defined as: a) subjects who received at least 80% of the total number of doses during the study; b) subjects who had sufficient plasma sample data to assess maximal HCV RNA suppression over the 7-day treatment period; and who also have sufficient plasma sample data to assess steady-state exposure parameters in plasma (AUC_{0-τ}, Cₘₐₓ, Cₘᵢₙ) on Day 7. Data analysis was performed using a validated version of WinNonlin® Enterprise (Version 5.2) software, and summary tables and figures were generated using a validated version of WinNonlin® Autopilot™ (Version 1.1.1) and other reporting tools, including SigmaPlot®.

### SAD Study (Part A)

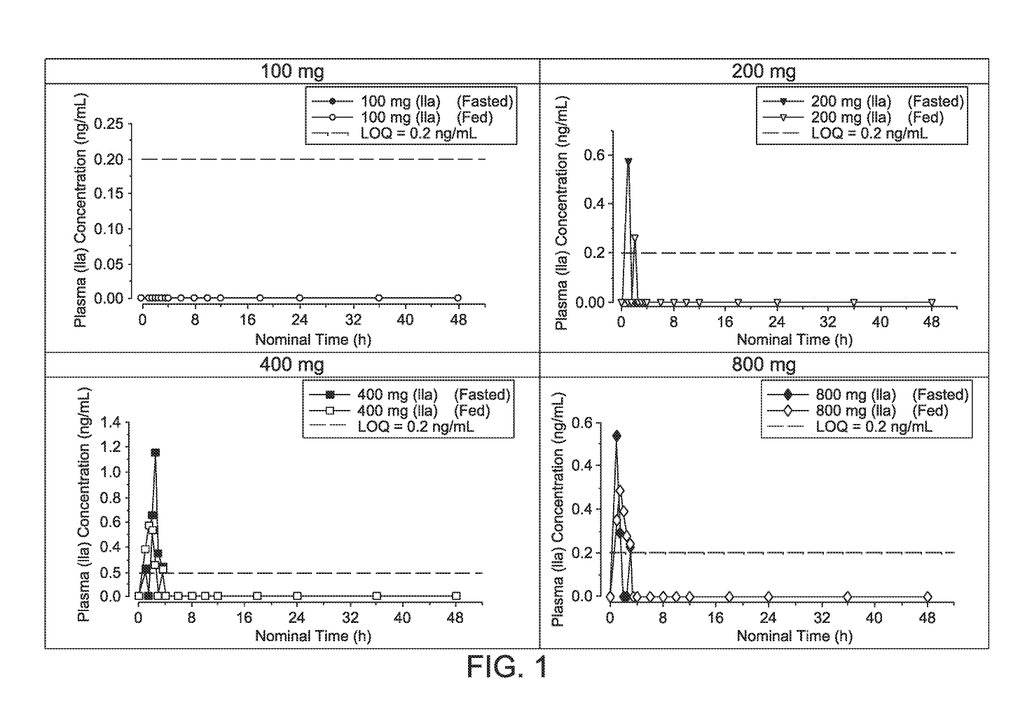

Individual Formula (IIa) plasma concentration data following a single oral administration of Formula (IIa) on Day 1 under fasted or fed conditions are presented in FIG. 1. Following a single oral dose of Formula (IIa), there were no measurable Formula (IIa) concentrations in the lowest dose group (100 mg Formula (IIa)) under both fasting and fed conditions, while there was only one non-BLQ (below the limit of quantitation) value per subject observed in the 200 mg Formula (IIa) dose group. At the 400 and 800 mg dose levels, peak levels were observed within 1 to 2.5 hours and mean plasma concentrations of Formula (IIa) on Day 1 declined rapidly thereafter, falling to BLQ after 3 to 4 hours post dose. Thus, the plasma concentration-time profile of Formula (IIa) was limited to a maximum of 4 hours post dose. Individual plasma Formula (IIa) PK parameters are presented below in Table 7.

TABLE 7
Mean Plasma PK Parameters of Formula (IIa) after Single Oral Administration
of Formula (IIa) under Fasting and Fed Conditions.

| PK | 100 mg | | 200 mg | | 400 mg | | 800 mg | |
|---|---|---|---|---|---|---|---|---|
| Parameter | Fasted | Fed | Fasted | Fed | Fasted | Fed | Fasted | Fed |
| N | 1 | 1 | 1 | 1 | 1 | 1 | 1 | 1 |
| AUC₀₋ₜ | NC | NC | 0.289 | 0.0653 | 1.31 | 1.07 | 0.751 | 0.900 |
| (ng · h/ml) | | | | | | | | |
| AUC₀₋₂₄ | NC | NC | 0.433 | 0.131 | 1.37 | 1.13* | 0.809 | 0.960* |
| (ng · h/ml) | | | | | | | | |
| AUC_{0-∞} | NC | NC | NC | NC | NC | 1.43* | NC | 1.39* |
| (ng · h/ml) | | | | | | | | |
| Cₘₐₓ | NC | NC | 0.577 | 0.261 | 1.16 | 0.574 | 0.734 | 0.486 |
| (ng/ml) | | | | | | | | |
| Tₘₐₓ | NC | NC | 1.00 | 2.00 | 2.50 | 1.50 | 1.00 | 1.50 |
| (h) | | | | | | | | |
| t_{1/2} | NC | NC | NC | NC | NC | 1.16* | NC | 1.42* |
| (h) | | | | | | | | |

Note:
CV % for the mean was not presented. In Part A, only one subject was dosed in the fasted and fed states per dose level.
*% AUCinf ext >20% (i.e. rounding to the nearest integer; values <20.5% are rounded down and values ≤20.5% are rounded up); excluded from the calculation of summary statistics.
NC = not calculated

At the 400 mg and 800 mg Formula (IIa) dose groups, peak plasma concentrations in the fasted subjects were 2.0 and 1.5 times higher, respectively, compared with the fed subjects. Individual Tₘₐₓ ranges for subjects dosed under fasted and fed conditions were similar (i.e., 1.0 to 2.5 h vs. 1.5 to 2.0 h respectively). Overall, AUC₀₋ₜ was comparable between fed and fasted groups, with the exception of the 200 mg dose, where AUC₀₋ₜ in the fasted subject was 4-fold higher than the fed subject. The terminal phase was only discernible in two fed subjects (one in the 400 mg dose cohort and one in the 800 mg dose cohort), and was associated with a short half-life ranging from 1.16 h to 1.42 h.

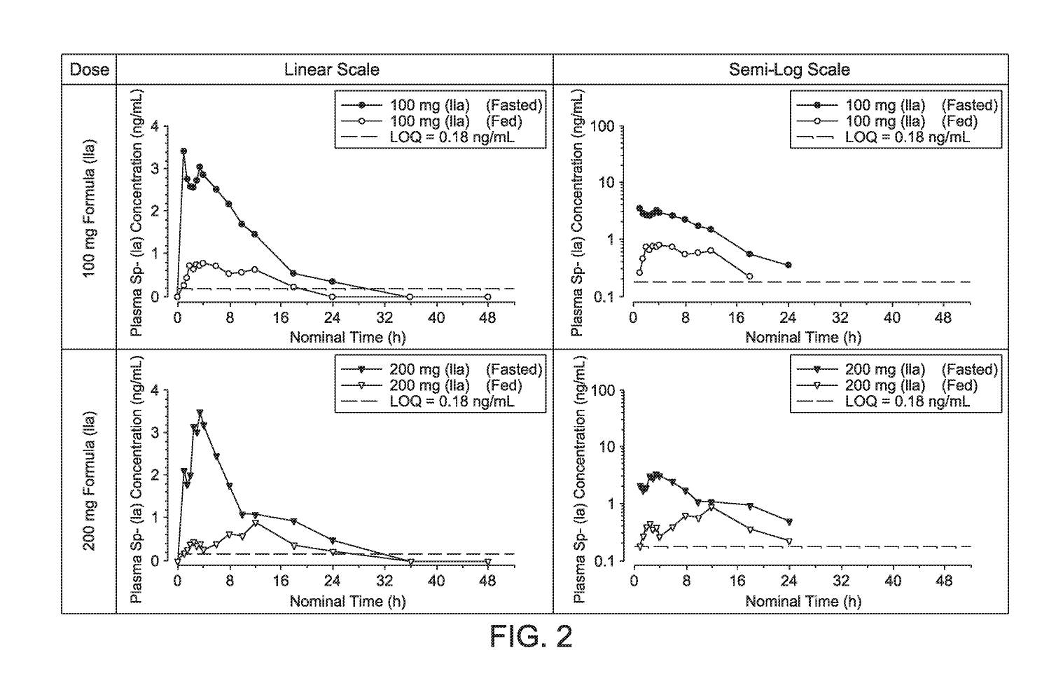

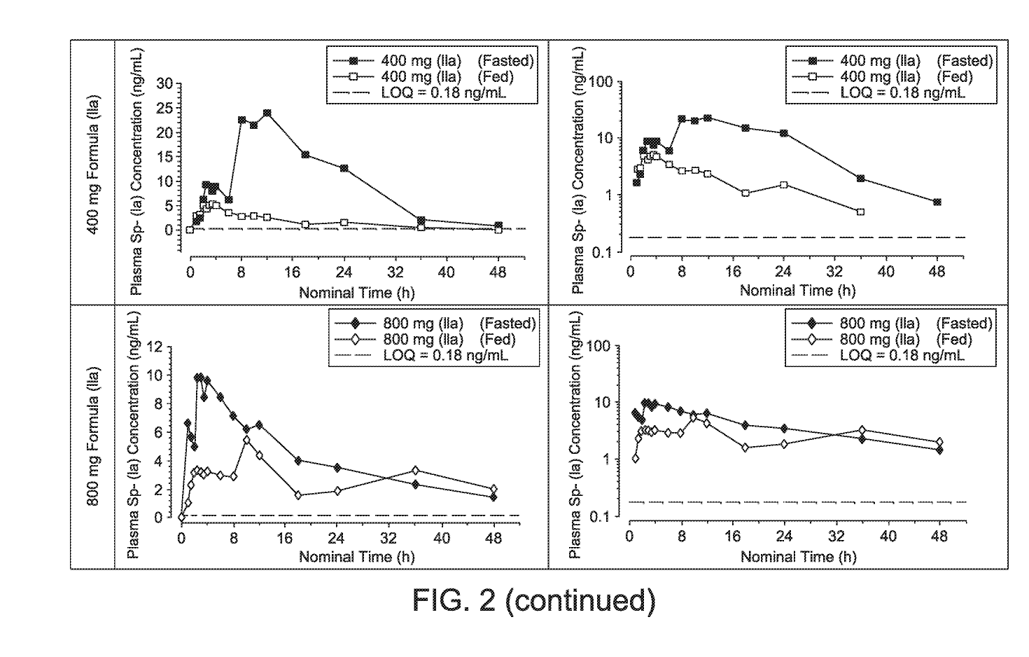

Individual Sp-isomer plasma concentration data following single oral administrations of Formula (IIa) on Day 1 under fed or fasted conditions are presented in Table 8 below. Mean Sp-isomer plasma concentration-time profiles stratified by dose level under fed or fasted conditions are presented in FIG. 2.

TABLE 8
Mean Plasma Pharmacokinetic Parameters of Sp-isomer after Single Oral
Administration of Formula (IIa) under Fasting and Fed Conditions.

| PK | 100 mg | | 200 mg | | 400 mg | | 800 mg | |
|---|---|---|---|---|---|---|---|---|
| Parameter | Fasted | Fed | Fasted | Fed | Fasted | Fed | Fasted | Fed |
| N | 1 | 1 | 1 | 1 | 1 | 1 | 1 | 1 |
| AUC₀₋ₜ | 35.6 | 9.53 | 34.5 | 11.1 | 461 | 70.4 | 199 | 131 |
| (ng · h/ml) | | | | | | | | |
| AUC₀₋₂₄ | 35.6 | 10.2 | 34.5 | 11.1 | 356 | 58.3 | 141 | 68.1 |
| (ng · h/ml) | | | | | | | | |
| AUC_{0-∞} | 38.5 | NC | 40.9 | NC | 468 | 78.7 | 240 | NC |
| (ng · h/ml) | | | | | | | | |
| Cₘₐₓ | 3.40 | 0.765 | 3.50 | 0.899 | 24.0 | 5.16 | 9.83 | 5.47 |
| (ng/ml) | | | | | | | | |
| Tₘₐₓ | 1.00 | 4.00 | 3.50 | 12.0 | 12.0 | 3.50 | 2.50 | 10.0 |
| (h) | | | | | | | | |
| t_{1/2} | 5.88 | NC | 9.00 | NC | 6.35 | 11.5 | 19.3 | NC |
| (h) | | | | | | | | |

In the 100 mg, 200 mg, 400 mg, and 800 mg dose groups, peak plasma concentrations in the fasted subjects were 4.4, 3.9, 4.7, and 1.8 times higher, respectively, compared to the fed subjects. Of note, Sp-isomer exposure appeared to be much higher for the 400 mg groups under fasted conditions, compared to the other treatment groups. The terminal phase was only discernible in one fed subject (400 mg dose), and was associated with a half-life of 11.5 h. The terminal half-life for fasted subjects ranged from 5.9 to 19.3 h.

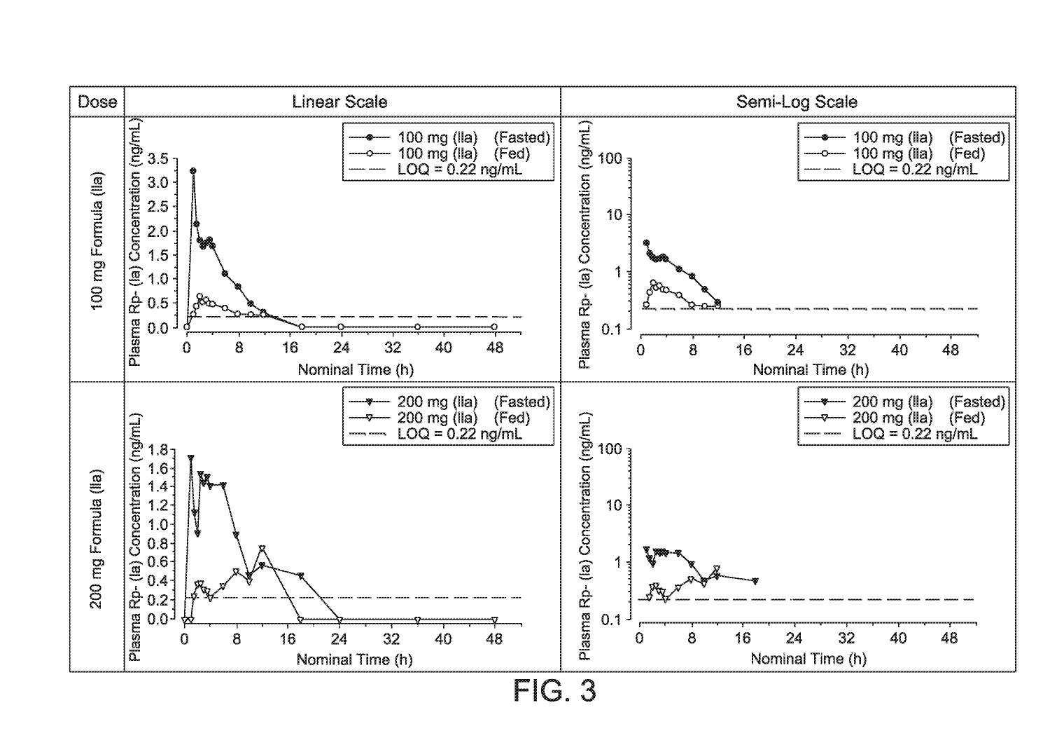

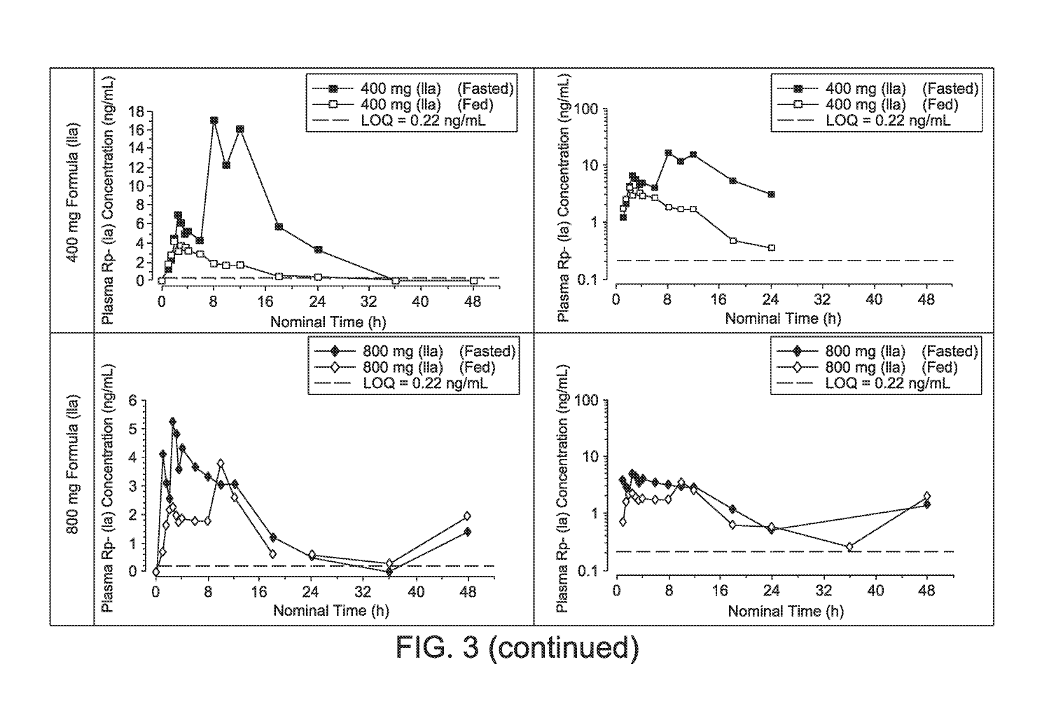

Individual Rp-isomer plasma concentration data following single oral administrations of Formula (IIa) on Day 1 under fed or fasted conditions are presented in Table 9 below. Mean Rp-isomer plasma concentration-time profiles stratified by dose level under fed or fasted conditions are presented in FIG. 3.

TABLE 9
Mean Plasma Pharmacokinetic Parameters of Rp-isomer after Single Oral
Administration of Formula (IIa) under Fasting and Fed Conditions

| PK | 100 mg | | 200 mg | | 400 mg | | 800 mg | |
|---|---|---|---|---|---|---|---|---|
| Parameter | Fasted | Fed | Fasted | Fed | Fasted | Fed | Fasted | Fed |
| N | 1 | 1 | 1 | 1 | 1 | 1 | 1 | 1 |
| AUC₀₋ₜ | 14.1 | 4.08 | 15.5 | 4.35 | 196 | 37.4 | 71.9 | 57.3 |
| (ng · h/ml) | | | | | | | | |
| AUC₀₋₂₄ | 15.0 | 4.82* | 16.9 | 6.62 | 195 | 37.4 | 60.0 | 38.9 |
| (ng · h/ml) | | | | | | | | |
| AUC_{0-∞} | 15.2 | 6.87* | NC | NC | 221 | 40.5 | NC | NC |
| (ng · h/ml) | | | | | | | | |
| Cₘₐₓ | 3.22 | 0.623 | 1.71 | 0.757 | 17.1 | 4.17 | 5.25 | 3.83 |
| (ng/ml) | | | | | | | | |
| Tₘₐₓ | 1.00 | 2.00 | 1.00 | 12.0 | 8.00 | 2.00 | 2.50 | 10.0 |
| (h) | | | | | | | | |
| t_{1/2} | 2.62 | 7.84* | NC | NC | 5.37 | 5.86 | NC | NC |
| (h) | | | | | | | | |

For the 100 mg, 200 mg, 400 mg, and 800 mg dose groups, peak plasma concentrations in the fasted subjects were 5.2, 2.3, 4.1, and 1.4 times higher, respectively, compared to the fed subjects. AUC₀₋₂₄ was 3.1, 2.6, 5.2, and 1.5 times higher under fasted conditions relative to fed conditions at the 100 mg, 200 mg, 400 mg, and 800 mg dose levels, respectively. Of note, Rp-isomer exposure appeared to be much higher for the 400 mg groups under fasted conditions, compared to the other treatment groups. The terminal half-life could only be assessed for the 100 mg and 400 mg dose levels, and ranged from 2.62 h to 7.84 h.

Overall, a single oral administration of Formula (IIa) at dose levels of 100 mg and 200 mg did not produce measurable concentrations of Formula (IIa) in plasma. At the 400 mg and 800 mg dose levels, peak levels were observed within 1 to 1.5 hours and mean plasma concentrations of Formula (IIa) on Day 1 declined rapidly thereafter, falling to BLQ after 3 to 4 hours post dose. Thus, the plasma concentration-time profile of Formula (IIa) was limited to a maximum of 4 hours post dose. This did not allow for a detailed pharmacokinetic analysis of single dose Formula (IIa) across the dose range studied. Metabolite Sp-isomer and Rp-isomer formation was rapid, with measurable concentrations in plasma that were at the first post dose blood draw (i.e., 1 hour post dose) across all subjects. Multiple peaks were observed in the individual concentration-time profiles of Formula (IIa) and its metabolites Sp-isomer and Rp-isomer in plasma.

There were no clear differences in parent and metabolite Tₘₐₓ between fed and fasted groups in the single dose study. In general, the rate and extent of exposure of the parent compound and metabolite, as assessed with Cₘₐₓ and AUC, appeared higher in the fasted subjects relative to the fed subjects.

### MAD Study (Part B)

Following single and multiple oral dose administration of Formula (IIa) QD for 7 days, individual predose levels of Formula (IIa) on Day 1 and Day 7 were all BLQ. Peak Formula (IIa) levels were observed within 1 to 1.7 hours, after which mean plasma concentrations of Formula (IIa) on Day 1 and Day 7 declined rapidly in an apparent monoexponential manner over the 200 to 400 mg Formula (IIa) QD dose range, while Formula (IIa) declined in an apparent biexponential manner at the 900 mg Formula (IIa) QD dose level. The mean plasma concentration-time profile of Formula (IIa) was limited to a maximum of 6 hours post dose, and individual plasma concentrations of Formula (IIa) were BLQ after 18 hours post-dose over the dose range studied. For most individuals where sufficient non-zero plasma concentrations of Formula (IIa) were available to characterize the concentration-time profile of Formula (IIa) in plasma, smaller secondary peaks, or shoulders, in the plasma concentration-time profile were observed approximately 2.5 to 6 h after Formula (IIa) administration. Mean (+SD) trough plasma concentration-time profiles of Formula (IIa) following multiple once-daily oral administrations of Formula (IIa) are presented in FIG. 5, and summary results are presented in Table 10.

The time to peak plasma Formula (IIa) concentration levels were similar after single or multiple oral Formula (IIa) administration, with median Tₘₐₓ ranging from 1.00 to 1.51 h on Day 1 and 1.00 to 1.28 h on Day 7. Mean peak plasma concentrations and AUC₀₋₂₄ were consistently higher on Day 7 (mean Cₘₐₓ range: 0.885 to 6.66 ng/mL and mean AUC₀₋₂₄ range: 0.770 to 13.7 ng·h/mL) compared to Day 1 (mean Cₘₐₓ range: 0.531 to 3.75 ng/mL and mean AUC₀₋₂₄ range: 0.512 to 7.91 ng·h/mL). Mean plasma Formula (IIa) t_{1/2} were comparable between Day 1 and Day 7 (i.e, 0.963 to 1.05 h and 0.684 to 1.07 h, respectively) for the 400 mg and 900 mg QD doses. Mean R(AUC) at the 200 mg dose level could not be calculated due to the lack of measurable concentrations or very low plasma concentrations of Formula (IIa). The mean accumulation factor based on Cₘₐₓ across dose levels for Formula (IIa) ranged from 1.43 to 2.94.

TABLE 10
Arithmetic Mean (CV %) Plasma Pharmacokinetic Parameters of Formula (IIa) after Single
and Multiple Once-Daily Oral Administration of Formula (IIa) under Fasting Conditions.

| PK | Day 1 | | | Day 7 | | |
|---|---|---|---|---|---|---|
| Parameter | 200 mg | 400 mg | 900 mg | 200 mg | 400 mg | 900 mg |
| N | 5^{a} | 6 | 10 | 6 | 6 | 10 |
| AUC₀₋ₜ | 0.393 (97.0) | 3.27 (125.0) | 5.31 (93.6) | 1.41 (129.5) | 3.10 (43.3) | 9.39 (185.2) |
| (ng · h/ml) | | | | | | |
| AUC₀₋₂₄^{b} | 0.512 (NC)^{d} | 7.91 (67.4)^{d} | 4.58 (99.0)^{d} | 0.770 (76.3)^{d} | 2.71 (48.4)^{d} | 13.7 (161.6)^{d} |
| (ng · h/ml) | | | | | | |
| AUC_{0-∞} | NC (NC) | 8.04 (61.1)^{d} | 7.25 (65.5)^{d} | NC | NC | NC |
| (ng · h/ml) | | | | | | |
| Cₘₐₓ | 0.531 (43.6) | 2.13 (127.0) | 3.75 (106.0) | 0.885 (85.4) | 2.52 (54.8) | 6.66 (219.6) |
| (ng/ml) | | | | | | |
| Tₘₐₓ^{c} | 1.00 (1.00, | 1.51 (1.02, | 1.04 (0.950, | 1.00 (1.00, | 1.28 (1.00, | 1.25 (0.817, |
| | 1.50) | 3.00) | 2.02) | 1.67) | 2.00) | 2.00) |
| (h) | | | | | | |
| Cₘᵢₙ | NA | NA | NA | 0.00 (NC) | 0.00 (NC) | 0.00 (NC) |
| (ng/ml) | | | | | | |
| Tₘᵢₙ^{c} | NA | NA | NA | 0.00 (0.00, | 0.00 (0.00, | 0.00 (0.00, |
| (h) | | | | 0.00) | 0.00) | 0.00) |
| t_{1/2} | NC (NC) | 1.05 (84.0)^{d} | 0.963 (27.5)^{d} | NC (NC) | 0.684 (40.1)^{d} | 1.07 (44.2)^{d} |
| (h) | | | | | | |
| R(AUC) | NA | NA | NA | NC (NC) | 0.246 (NC)^{d} | 1.25 (71.5)^{d} |
| R(Cₘₐₓ) | NA | NA | NA | 2.61 (80.2)^{a,d} | 2.94 (91.3) | 1.43 (106.1) |

NA = Not applicable; NC = Not calculated
^{a}Plasma concentrations of SB 9200 were all BLQ for Subject 33M103 on Day 1.
^{b}AUC₀₋₂₄ = AUC₀₋ₜ on Day 7
^{c}Median (Min, Max)
^{d}There are less subjects included in the statistics for this parameter than the N presented for this group. The concentration-time profile did not exhibit a terminal log-linear phase or the % of AUC_{0-∞} extrapolated from AUC₀₋ₜ exceeded 20% (i.e., rounding to the nearest integer; values <20.5% are rounded down and values ≥20.5% are rounded up) for some subjects. The PK parameters AUC₀₋₂₄, AUC_{0-∞}, and t_{1/2} were not calculated for some subjects.

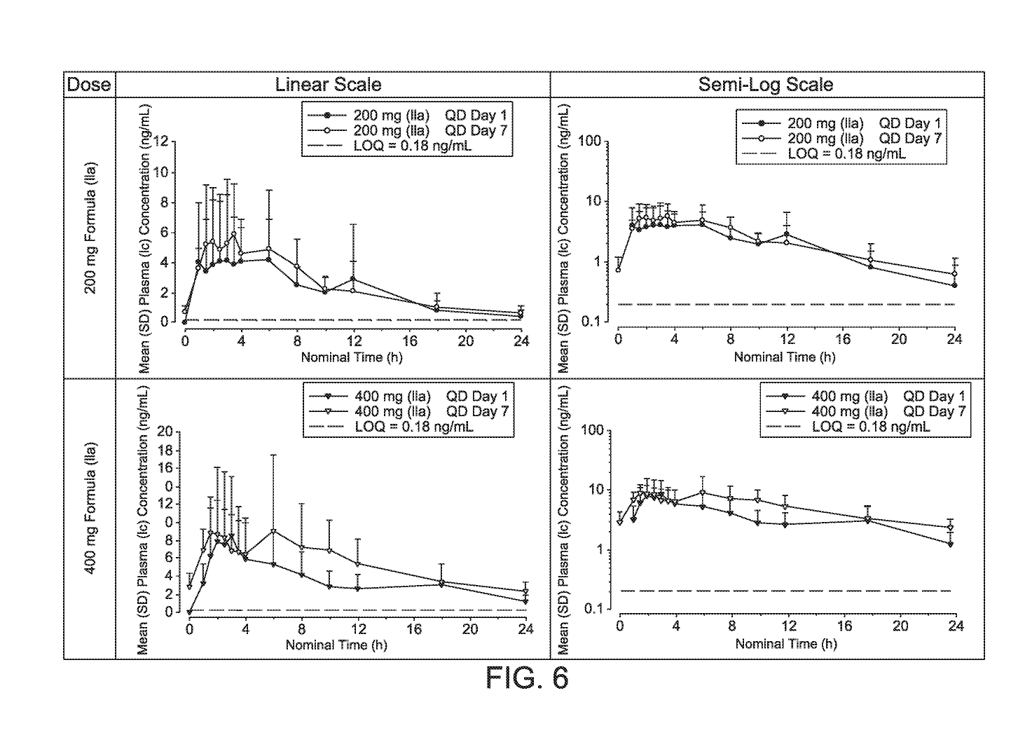

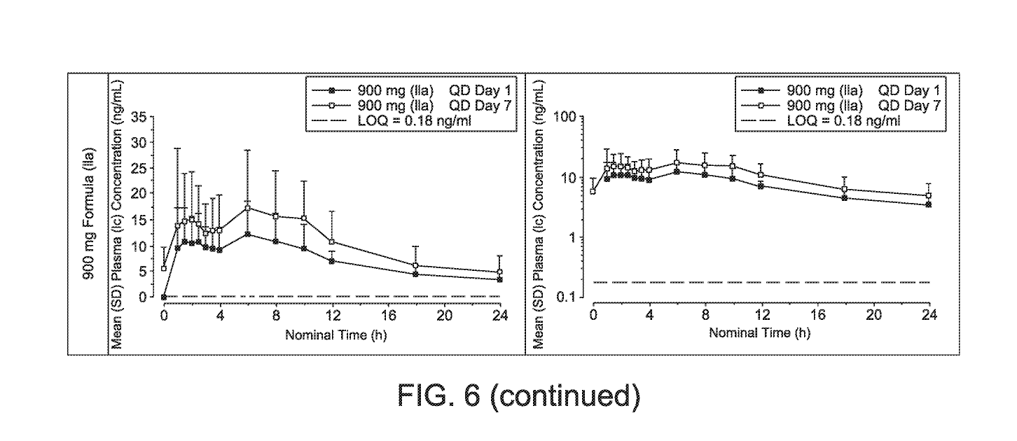

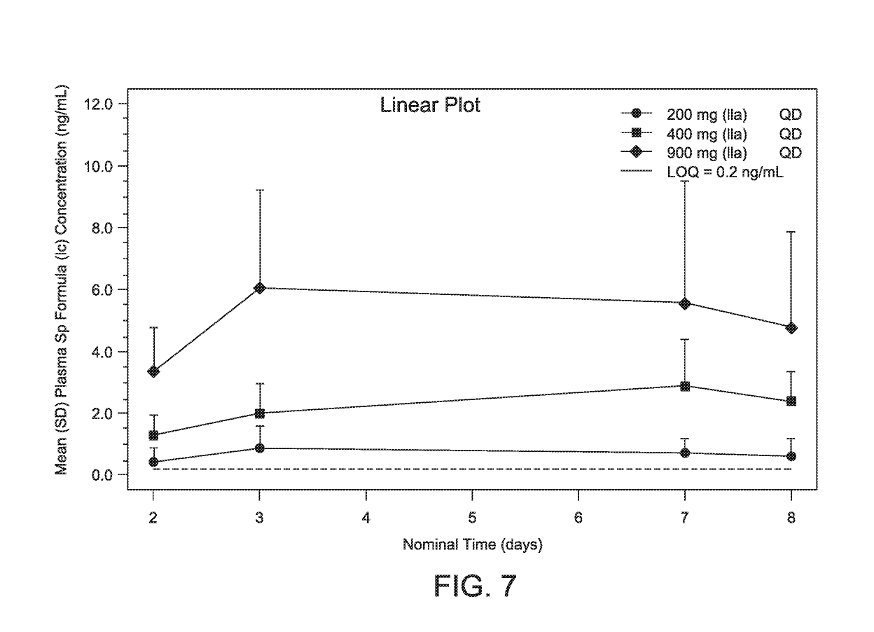

Mean (+SD) plasma concentration-time profiles of the Sp-isomer following single and multiple once-daily oral administrations of Formula (IIa) are presented in FIG. 6, and mean (+SD) plasma concentration-time profiles of the Sp-isomer following multiple once-daily oral administrations of Formula (IIa) are presented in FIG. 7.

Mean plasma trough levels of metabolite Sp-isomer following multiple once-daily oral administration of Formula (IIa) under fasted conditions were increased with increasing Formula (IIa) dose. Plasma trough Sp-isomer concentrations from all dose levels suggest that steady state was probably attained by Day 3 of dosing. Summary results of the Sp-isomer PK parameters are presented below in Table 11.

TABLE 11
Arithmetic Mean (CV %) Plasma Pharmacokinetic Parameters of the Sp-isomer after Single
and Multiple Once-Daily Oral Administration of Formula (IIa) under Fasting Conditions.

| PK | Day 1 | | | Day 7 | | |
|---|---|---|---|---|---|---|
| Parameter | 200 mg | 400 mg | 900 mg | 200 mg | 400 mg | 900 mg |
| N | 6 | 6 | 10 | 6 | 6 | 10 |
| AUC₀₋ₜ | 52.7 (56.6) | 81.6 (46.6) | 168 (33.2) | 60.5 (56.0) | 129.1 (47.4) | 253 (51.5) |
| (ng · h/ml) | | | | | | |
| AUC₀₋₂₄^{a} | 52.0 (39.4)^{c} | 73.1 (81.5)^{c} | 177 (20.4)^{c} | 62.6 (59.7)^{c} | 147 (33.6)^{c} | 276 (57.2)^{c} |
| (ng · h/ml) | | | | | | |
| AUC_{0-∞} | 65.9 (12.2)^{c} | 82.9 (75.6)^{c} | 215 (29.6)^{c} | NA | NA | NA |
| (ng · h/ml) | | | | | | |
| Cₘₐₓ | 7.13 (58.4) | 10.6 (66.9) | 16.5 (37.0) | 7.76 (53.2) | 12.0 (58.1) | 22.0 (68.4) |
| (ng/ml) | | | | | | |
| Tₘₐₓ^{b} | 4.50 (2.50, | 4.32 (1.52, | 4.25 (1.00, | 2.50 (1.00, | 1.50 (1.00, | 4.23 (1.00, |
| (h) | 12.0) | 8.02) | 10.0) | 6.00) | 9.98) | 10.0) |
| Cₘᵢₙ | NA | NA | NA | 0.629 (82.0) | 2.25 (55.2) | 4.48 (72.3) |
| (ng/ml) | | | | | | |
| Tₘᵢₙ^{b} | NA | NA | NA | 0.00 (0.00, | 9.00 (0.00, | 0.00 (0.00, |
| (h) | | | | 24.00) | 24.00) | 24.00) |
| t_{1/2} | 5.20 (35.5)^{c} | 8.90 (37.2)^{c} | 7.87 (13.5)^{c} | 4.94 (20.3)^{c} | 8.55 (38.1)^{c} | 4.65 (15.5)^{c} |
| (h) | | | | | | |
| R(AUC) | NA | NA | NA | 1.39 (61.1)^{c} | 1.61 (20.9)^{c} | 1.47 (49.2)^{c} |
| R(Cₘₐₓ) | NA | NA | NA | 1.32 (53.9)^{c} | 1.53 (64.0) | 1.43 (77.6) |

NA = Not applicable; NC = Not calculated
^{a}AUC₀₋₂₄ = AUC₀₋ₜ on Day 7
^{b}Median (Min, Max)
^{c}There are less subjects included in the statistics for this parameter than the N presented for this group. The concentration-time profile did not exhibit a terminal log-linear phase or the % of AUC_{0-∞} extrapolated from AUC₀₋ₜ exceeded 20% (i.e., rounding to the nearest integer; values <20.5% are rounded down and values ≥20.5% are rounded up) for some subjects. The PK parameters AUC₀₋₂₄, AUC_{0-∞}, and t_{1/2} were not calculated for some subjects.

The time to peak plasma Sp-isomer concentration levels appeared slightly longer after single administration compared to multiple administration with the median Tₘₐₓ ranging from 4.32 to 7.51 h on Day 1 and from 1.50 to 4.23 h on Day 7. However, this is probably due to variability in the observation of multiple peaks in the PK profile and a limited set of data. Mean peak plasma concentrations were comparable between Day 7 (range: 7.76 to 22.0 ng/mL) and Day 1 (range: 7.13 to 16.5 ng/mL), however mean AUC₀₋₂₄ was only comparable at the 200 mg dose level (i.e., 52.0 vs. 62.6 ng·h/mL on Day 1 and Day 7, respectively). At the 400 mg and 900 mg dose levels, mean AUC₀₋₂₄ was 1.6- to 2.0-fold higher on Day 7 (AUC₀₋₂₄ ranged from 147 to 276 ng·h/mL) compared to Day 1 (AUC₀₋₂₄ ranged from 73.1 to 177 ng·h/mL). Mean plasma Sp-isomer t_{1/2} were comparable between Day 1 and Day 7 (i.e, 5.20 to 8.90 h and 4.65 to 8.55 h, respectively) over the dose range studied. The mean accumulation factors based on AUC and Cₘₐₓ across dose levels for the Sp-isomer ranged from 1.39 to 1.61 and from 1.32 to 1.53, respectively. Based on R(AUC), a low to moderate accumulation of the Sp-isomer was observed following repeated once-daily dosing for 7 days.

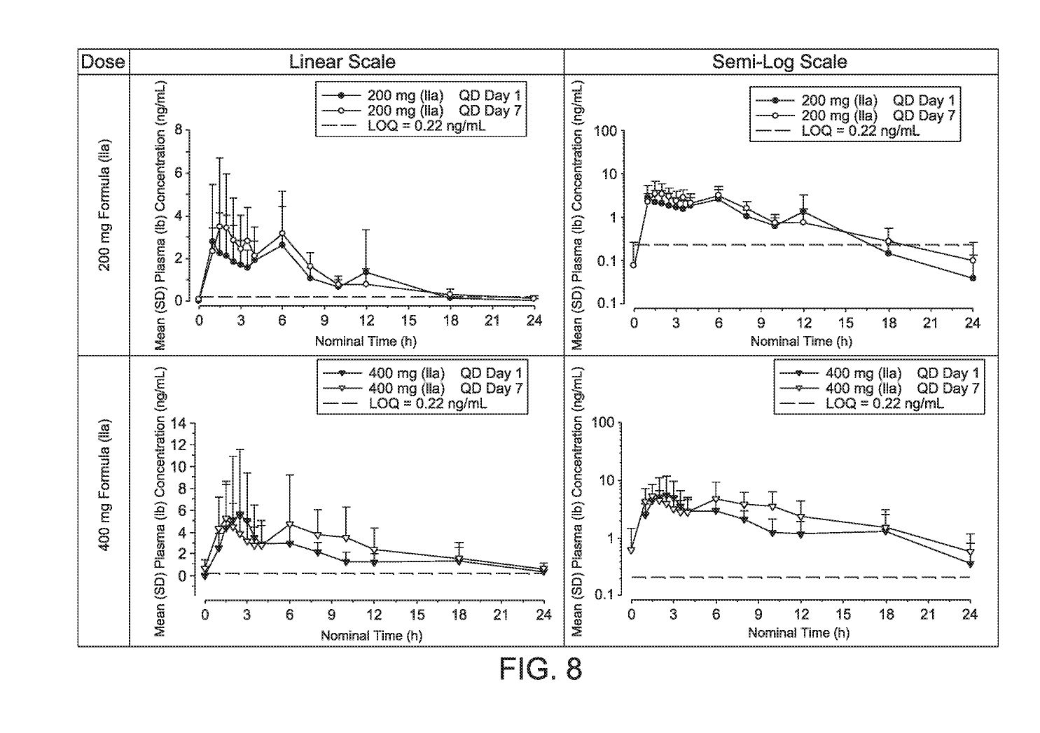

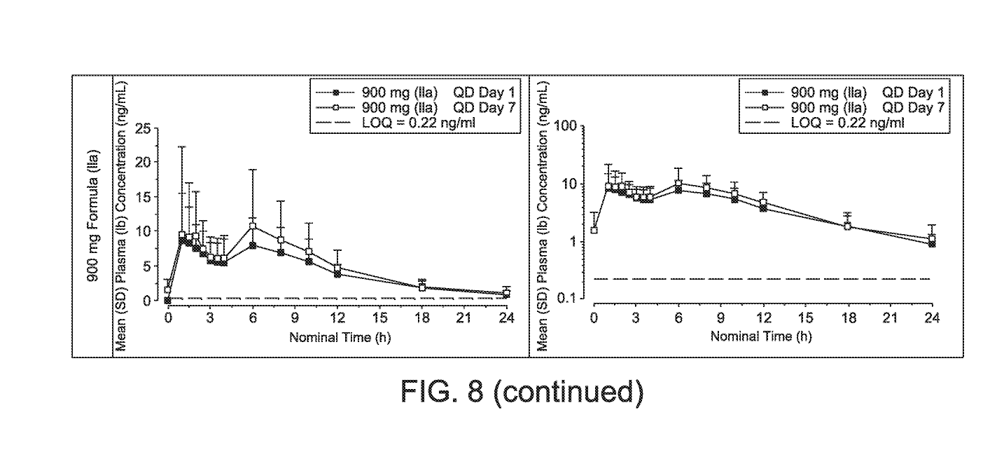

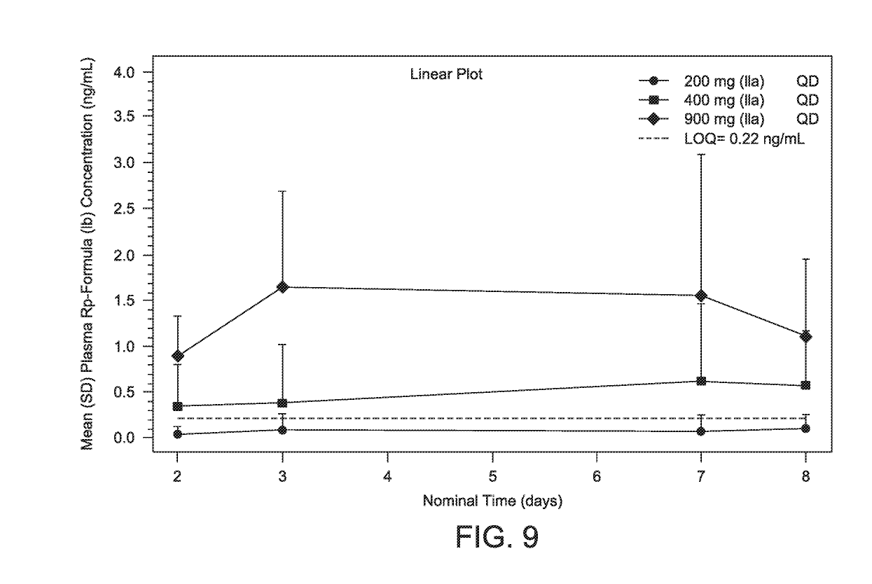

Mean (+SD) plasma concentration-time profiles of the Rp-isomer following single and multiple once-daily oral administrations of Formula (IIa) are presented in FIG. 8, and mean (+SD) plasma concentration-time profiles of the Rp-isomer following multiple once-daily oral administrations of Formula (IIa) are presented in FIG. 9.

Mean plasma trough levels of metabolite the Rp-isomer following multiple once-daily oral administration of Formula (IIa) under fasted conditions were below the LLOQ for the 200 mg Formula (IIa) QD treatment group, while trough levels increased when the Formula (IIa) dose was increased from 400 mg to 900 mg QD. Plasma trough Rp-isomer concentrations from all dose levels suggest that steady state was probably attained by Day 3 of dosing. Summary results of Rp-isomer PK parameters are presented below in Table 12.

TABLE 12
Arithmetic Mean (CV %) Plasma Pharmacokinetic Parameters of the Rp-isomer after Single
and Multiple Once-Daily Oral Administration of Formula (IIa) under Fasting Conditions.

| | Day 1 | | | Day 7 | | |
|---|---|---|---|---|---|---|
| PK Parameter | 200 mg | 400 mg | 900 mg | 200 mg | 400 mg | 900 mg |
| N | 6 | 6 | 10 | 6 | 6 | 10 |
| AUC₀₋ₜ | 24.4 (54.4) | 41.5 (54.6) | 99.3 (22.3) | 25.8 (42.2) | 60.3 (57.6) | 120 (59.2) |
| (ng · h/ml) | | | | | | |
| AUC₀₋₂₄^{a} | 24.1 (35.7)^{c} | 31.1 (45.0)^{c} | 104 (18.8)^{c} | 26.2 (45.7)^{c} | 60.5 (57.2)^{c} | 133 (55.1)^{c} |
| (ng · h/ml) | | | | | | |
| AUC_{0-∞} | 22.2 (27.4)^{c} | 32.9 (38.8)^{c} | 113 (13.1)^{c} | NA | NA | NA |
| (ng · h/ml) | | | | | | |
| Cₘₐₓ | 4.32 (54.0) | 6.80 (72.3) | 12.6 (37.9) | 4.85 (56.0) | 7.58 (46.2) | 14.7 (82.7) |
| (ng/ml) | | | | | | |
| Tₘₐₓ^{b} | 4.50 (1.00, | 2.56 (1.52, | 5.04 (1.00, | 2.00 (1.50, | 3.77 (1.00, | 4.23 (1.00, |
| (h) | 12.0) | 8.02) | 10.0) | 6.00) | 9.98) | 10.0) |
| Cₘᵢₙ | NA | NA | NA | 0.0743 (244.9) | 0.379 (132.5) | 1.12 (103.2) |
| (ng/ml) | | | | | | |
| Tₘᵢₙ^{b} | NA | NA | NA | 0.00 (0.00, | 0.00 (0.00, | 0.00 (0.00, |
| (h) | | | | 0.00) | 24.00) | 24.00) |
| t_{1/2} | 4.94 (47.6)^{c} | 5.94 (41.8)^{c} | 4.78 (30.0)^{c} | 4.98 (78.4)^{c} | 5.94 (12.3)^{c} | 4.31(32.2)^{c} |
| (h) | | | | | | |
| R(AUC) | NA | NA | NA | 1.50 (62.2)^{c} | 1.51 (25.5)^{c} | 1.24 (56.7)^{c} |
| R(Cₘₐₓ) | NA | NA | NA | 1.27 (48.5)^{c} | 1.53 (66.7) | 1.11 (55.9) |

NA = Not applicable; NC = Not calculated
^{a}AUC₀₋₂₄ = AUC₀₋ₜ on Day 7
^{b}Median (Min, Max)
^{c}There are less subjects included in the statistics for this parameter than the N presented for this group. The concentration-time profile did not exhibit a terminal log-linear phase or the % of AUC_{0-∞} extrapolated from AUC₀₋ₜ exceeded 20% (i.e., rounding to the nearest integer; values <20.5% are rounded down and values ≥20.5% are rounded up) for some subjects. The PK parameters AUC₀₋₂₄, AUC_{0-∞}, and t_{1/2} were not calculated for some subjects.

The time to peak plasma Rp-isomer concentration levels were similar for single and multiple oral Formula (IIa) administrations, with median Tmax ranging from 2.56 to 5.04 h on Day 1 and 2.00 to 4.23 h on Day 7. Mean peak plasma concentrations across all dose levels were comparable between Day 7 (range: 4.85 to 14.7 ng/mL) and Day 1 (range: 4.32 to 12.6 ng/mL), however mean AUC₀₋₂₄ was only comparable at the 200 mg dose level (i.e., 24.1 vs. 26.2 ng·h/mL on Day 1 and Day 7, respectively). At the 400 and 900 mg dose levels, mean AUC₀₋₂₄ was 95 and 27% higher, respectively, on Day 7 (AUC₀₋₂₄ ranged from 60.5 to 133 ng·h/mL) compared to Day 1 (AUC₀₋₂₄ ranged from 31.1 to 104 ng·h/mL). Mean plasma Rp-isomer t_{1/2} were comparable between Day 1 and Day 7 (i.e, 4.78 to 5.94 h and 4.31 to 5.94 h, respectively) over the dose range studied. The mean accumulation factors based on AUC and Cₘₐₓ across dose levels for Rp-isomer ranged from 1.24 to 1.51 and from 1.11 to 1.53, respectively. Based on R(AUC), a low to moderate accumulation of Rp-isomer was observed following repeated once-daily dosing for 7 days.

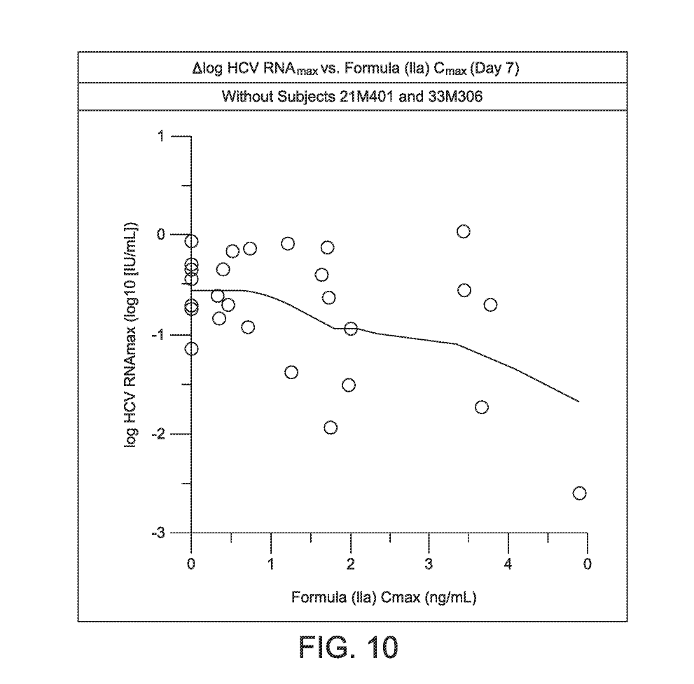

A scatter plot correlation matrix of steady state Formula (IIa) Cₘₐₓ on Day 7 vs. maximum suppression of HCV RNA (Δ log HCV RNAₘₐₓ) after exclusion of extreme Formula (IIa) values for two subjects is presented in FIG. 10. Extreme Formula (IIa) Cₘₐₓ values at steady state for two subjects were excluded from the exploratory PK/PD analysis in order to better visualize the relationship between the Formula (IIa) Cₘₐₓ values at steady state and maximum suppression of HCV RNA. A statistically significant relationship between the Formula (IIa) Cₘₐₓ at steady state and maximum suppression of HCV RNA on Day 7 was observed after exclusion of these extreme values. There were no other discernible relationships between plasma Formula (IIa) exposure parameters AUC₀₋ₜ, AUC_{0-τ}, and Cₘᵢₙ vs. maximum suppression of HCV RNA, nor were any relationships observed for the metabolites Sp-isomer and Rp-isomer in plasma.

Overall, following multiple once daily oral dose administration of Formula (IIa) QD for 7 days under fasted conditions, mean trough levels were all BLQ. Plasma trough concentrations of metabolites Sp-isomer and Rpisomer increased with increasing dose, and suggested that steady state was probably attained by Day 3 of dosing. On Day 7, the mean plasma concentration-time profile of Formula (IIa) was also limited to a maximum of 4 to 6 hours post dose over the dose range studied, as individual plasma concentrations of Formula (IIa) were all BLQ after 1.5 hours to 6 hours post dose.

Based on dose-normalized AUC₀₋ₜ and Cₘₐₓ, and taking into consideration the high intersubject variability in the exposure of Formula (IIa) and metabolites Sp-isomer and Rp-isomer, increases in AUC₀₋ₜ and Cₘₐₓ appeared to be dose proportional when Formula (IIa) oral doses were increased from 200 to 900 mg. The Formula (IIa) Cₘₐₓ ranged from 0.531 to 6.66 ng/mL at 0.817 and 3.00 hours. The Sp-isomer Cₘₐₓ ranged from 7.13 to 22.0 ng/mL at 1.00 and 12.0 hours, while the Rp-isomer Cₘₐₓ ranged from 4.32 to 14.7 ng/mL at 1.00 and 12.0 hours, respectively. Peak individual viral load drop improved from 1.5 to 1.9 log₁₀ when the dose increased from 200 mg to 400 mg. The terminal half-life (t_{1/2}) was relatively short for Formula (IIa), ranging from 0.684 hours to 1.07 hours, while the terminal half-life was slightly longer for the metabolites. Mean plasma Sp-isomer terminal half-life ranged from 4.65 hours to 8.9 hours on Days 1 and 7. Mean plasma Rp-isomer terminal half-life ranged from 4.31 hours to 5.94 hours on Days 1 and 7. These half-life values were generally consistent with the half-life values observed in Part A of the study (SAD study). In addition, the mean terminal half-life values for plasma Formula (IIa) and metabolites Sp-isomer and Rp-isomer were comparable between Day 1 and 7.

Based on the mean accumulation factor R(AUC) across dose levels for Sp-isomer (i.e., from 1.39 to 1.61) and Rp-isomer (i.e., 1.24 to 1.51), a low to moderate accumulation of Sp-isomer and Rp-isomer was observed following repeated once daily oral dosing of Formula (IIa) for 7 days.

Further, there were no discernible relationships between plasma Formula (IIa) steady state exposure parameters AUC₀₋ₜ, AUC_{0-τ}, and Cₘᵢₙ vs. maximum suppression of HCV RNA, nor were there any PK/PD relationships observed for the metabolites Sp-isomer and Rp-isomer. However, a statistically significant relationship between Cₘₐₓ at steady state and maximum suppression of HCV RNA on Day 7 was observed (p=0.015) after exclusion of extreme Cₘₐₓ values for Formula (IIa) and its metabolites for two subjects.

### Example 2. Administration of Formula (IIa) in Combination with Sofosbuvir

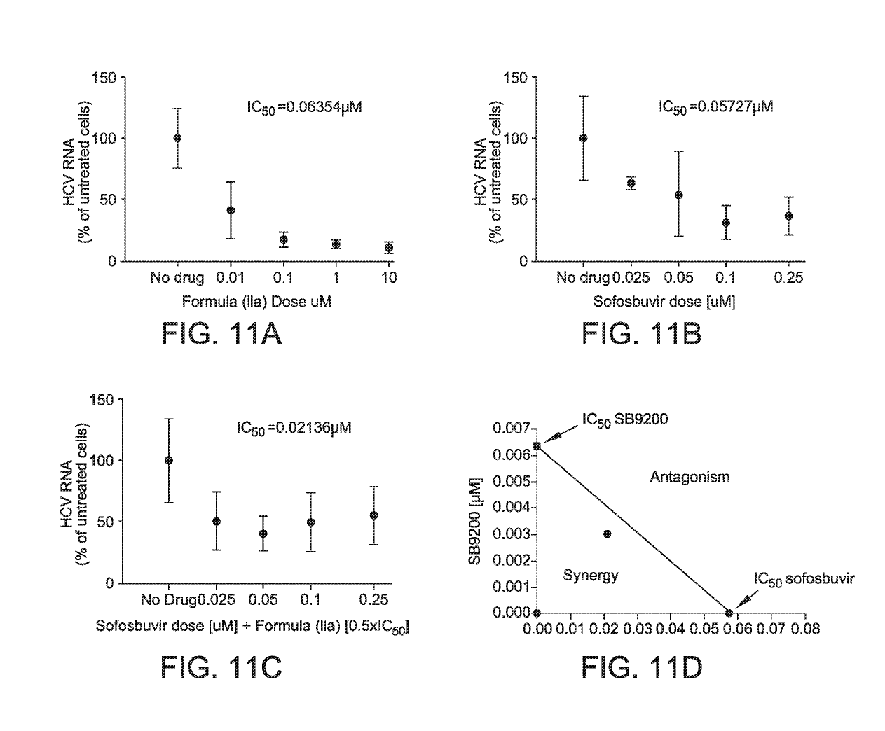

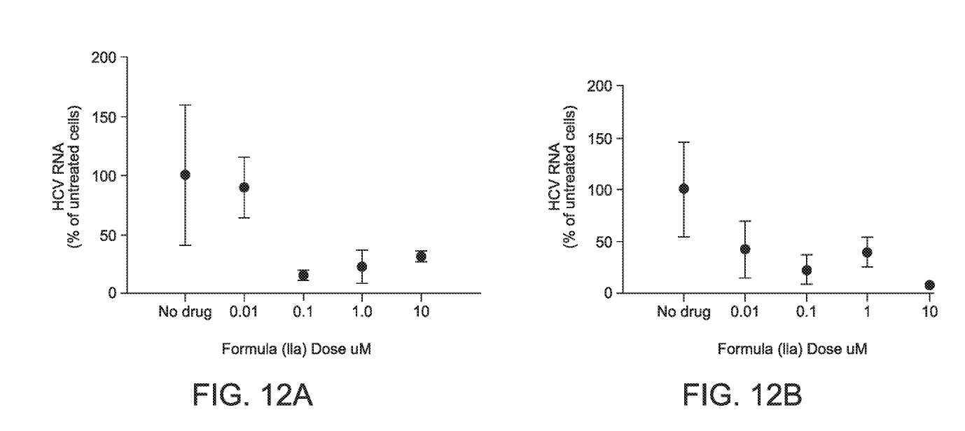

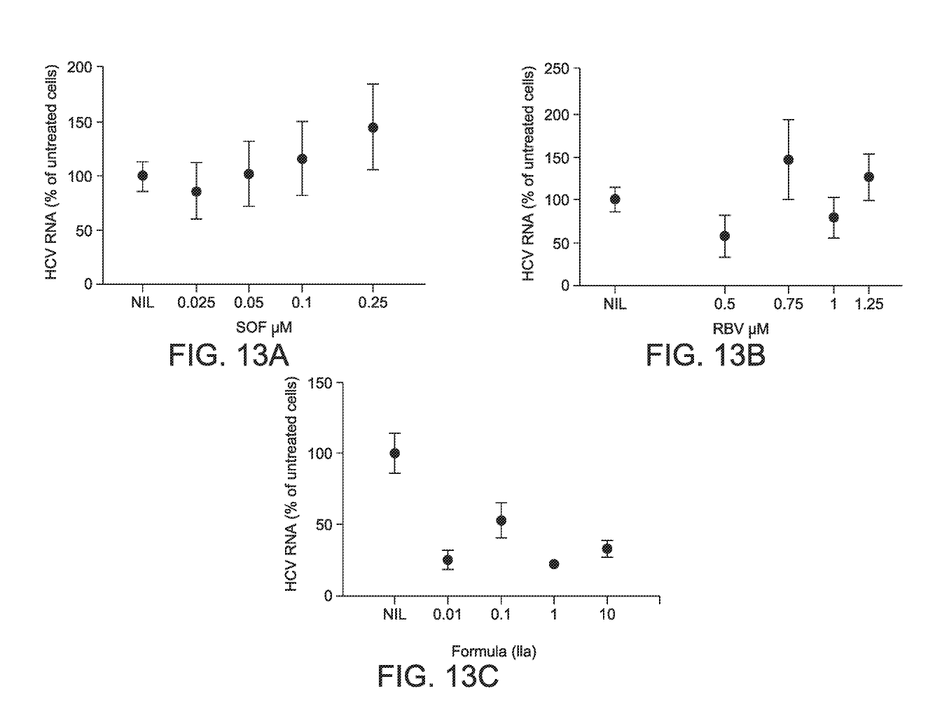

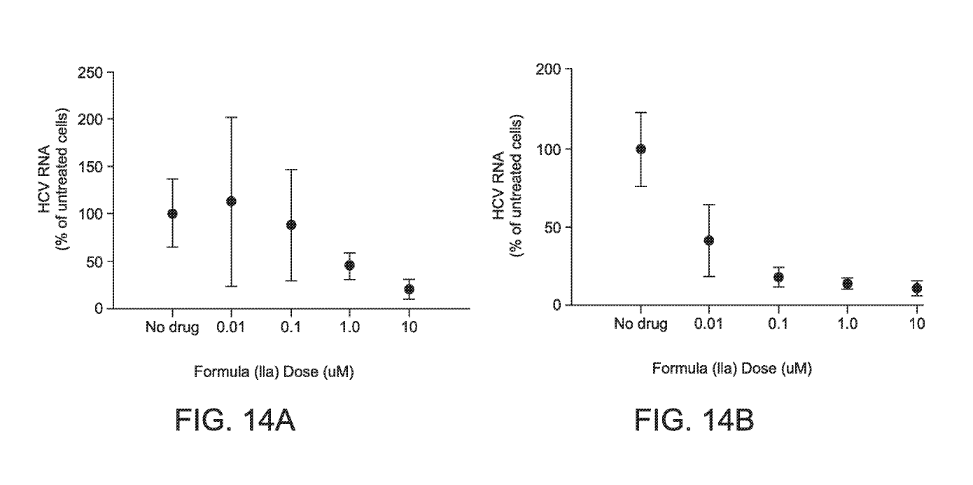

Antiviral activity of Formula (IIa) and sofosbuvir against HCV was assessed using a 3-day assay (Okuse, et al (2005) Antiviral Res 65:23; Korba et al (2008) Antiviral Res 77:56) in the stably-expressing HCV replicon cell line AVA5 [sub-genomic (CON1), genotype 1b] (Blight et al (2000) Science 290:1972) maintained as sub-confluent cultures on 96-well plates. Formula (IIa) and sofosbuvir were formulated as solutions in dimethylsulfoxide and dosed to cells at varying concentrations. Antiviral activity was determined by blot hybridization analysis of intracellular HCV RNA normalized to the level of cellular B-actin RNA in each culture sample. Cytotoxicity was assessed by neutral red dye uptake in cultures maintained in parallel plates. IC₅₀ values were calculated by linear regression analysis (MS Excel® and Quattropro®) using data combined from all treated cultures and are summarized in FIGS. 11A and 11B.

In order to test for synergism, the two agents were mixed together at predetermined relative ratios based on the IC₅₀ values of each compound. Five solutions were prepared in which Formula (IIa) was supplied at 0.5 times the determined IC₅₀ value (31.7 nM) and the concentration of sofosbuvir varied from 0 to 250 nM. The resulting IC₅₀ value of the combination was determined as described previously and is shown in FIG. 11C. Analysis of drug interactions in the combination study was determined through the use of the Calcusyn program (Biosoft, Inc., Cambridge, United Kingdom), which evaluates synergism, additivity, or antagonism. The isobologram resulting from this analysis is depicted in FIG. 11D.

### Example 3. Capture Fusion Assay to Determine the IC₅₀ of Formula (IIa) in Serum Samples from Patients Infected with Resistant Variants HCV

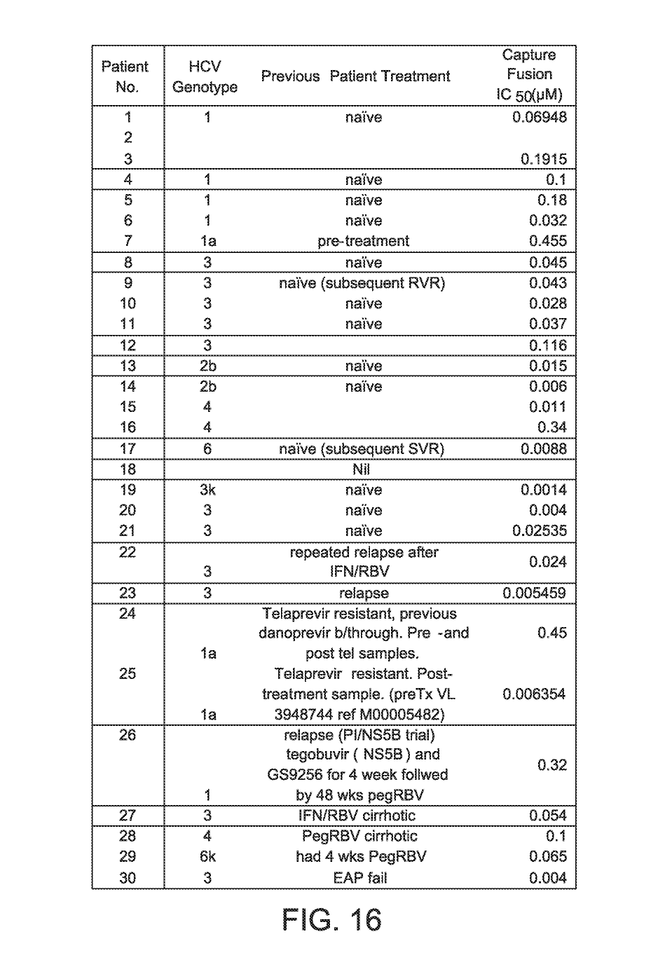

Serum samples were taken from 30 patients who had either responded to or previously failed treatment with combination peg-interferon/ribavirin and a direct acting antiviral (DAA), e.g., sofosbuvir and ledipasvir, or DAA treatment alone (FIG. 16). The anti-HCV activity of Formula (IIa) was assessed using the capture fusion assay. Briefly, pre-stimulated THP-1 cells were infected with serum from donors chronically infected with the common HCV genotypes G1 or G3, or from patients infected with the less prevalent G2, G4 or G6 HCV. The hybrid cells were treated once with a range of concentrations of Formula (IIa). For comparison, fused cells infected with G1 and G3 sera were treated with telaprevir or alisporivir. The cells were cultured for 5 days, before quantification of HCV RNA by PCR. Dose-response curves were used to calculate IC₅₀ values for each experiment. The detailed assay protocol is outlined below.

### Cells, Reagents and Clinical Material

Huh7.5 and Huh7 cells were maintained in Dulbecco's modified Eagle medium with glutamine supplemented with 10% fetal calf serum and 1% penicillin/streptomycin (DMEM/10% FCS/PS). THP-1 cells were maintained in RPMI with glutamine supplemented with 10% fetal calf serum and 1% penicillin/streptomycin (RPMI/10% FCS/PS). Peripheral blood mononuclear cells (PBMC) and sera were obtained from patients with chronic HCV infection, with informed consent. Cytokines used were phorbol 12-myristate 13-acetate (PMA, Sigma-Aldrich, Dorset, UK), IFNγ (Invitrogen, Paisley, UK) and osteopontin (R&D Systems, Abingdon, UK). Primary antibodies used were anti-albumin (Dako, Glostrup, Denmark), anti-CD81 (BD Biosciences, Oxford, UK), anti-SR-B1 (Novus Biologicals, Cambridge, UK), anti-CD32 and anti-CD64 (Abcam, Cambridge, UK). Mouse IgG1 isotype control was purchased from Biolegend UK (London, UK). Fluorescent secondary antibodies AlexaFluor488 and 594 anti-sheep, anti-mouse and anti-rabbit IgG were purchased from Invitrogen.

### Fusion of Patient Monocytes

PBMCs were separated from whole blood by centrifugation on Ficoll-Paque. The PBMC layer was washed twice before positive selection of CD14+ cells by magnetic separation. Huh7.5 cells were trypsinized and combined with CD14+ cells at a 1:1 ratio. After removal of all supernatant, the cell pellet was slowly resuspended in prewarmed polyethylene glycol 1500 (Roche Diagnostics, Burgess Hill, UK). After incubation at 37° C. for 2 minutes, prewarmed medium (DMEM/10% FCS/PS) was added dropwise and the cells washed by centrifugation. The fused cells were seeded into 6 well plates at a density of 5×10⁵ cells/mL and maintained at 37° C. until RNA extraction.

### Stimulation and Infection of THP-1 Cells

THP-1 cells were seeded into 6 well plates at a density of 10⁶ cells/mL and maintained in RPMI supplemented with 10% fetal calf serum and 1% penicillin/streptomycin (RPMI/10% FCS/1% PS) for 18 hours, with or without the addition of cytokines. The cells were washed three times and the medium replaced with RPMI/2% FCS and patient serum at a ratio of 1 HCV copy per cell. For antibody blocking experiments, cells were incubated with blocking antibody at 37° C. for one hour before addition of patient serum. After incubation at 37° C. for 18-24 hours, the supernatant was removed and cells were washed three times. Adherent cells were removed using a cell scraper and Huh7.5 cells added at a 1:1 ratio. Cell fusion was performed as described above. The fused cells were seeded into 6 well plates at a density of 10⁵ cells/mL and maintained at 37° C. in the presence or absence of antiviral drugs for up to 7 days. In selected experiments, supernatants were pooled from non-drug treated wells and concentrated on a sucrose gradient. In brief, the pooled supernatants were filtered through a 0.45 μm filter then 10 mL of each was layered on 4 mL 20% sucrose. The supernatants were centrifuged at 24,000×g for 2 hours and the resulting pellet resuspended in 1 mL RPMI. For capture-fusion experiments using concentrated supernatant, 10⁶ prestimulated THP-1 cells were incubated with 1 mL of each concentrated supernatant for 24 hours before fusion, as described above.

### Drug Inhibition Assays

Fused cells were rested overnight prior to addition of antiviral drugs. Telaprevir and other DAAs, as well as alisporivir were stored as 20 mM stock solutions in dimethyl sulfoxide (DMSO). Fresh dilutions were made for each experiment and each drug concentration was tested in quadruplicate. Drug dilution mix alone was added to the control wells (RPMI/2% FCS/0.05% DMSO). Medium and drug were refreshed at day 3 after fusion. After quantification of HCV RNA, viral RNA was calculated as a percentage of that present in the untreated wells. Dose-response curves were constructed and used to estimate the 50% inhibitory concentration (IC₅₀) of drug for each sample using Prism 4.0 software (GraphPad, La Jolla, Calif.).

### Results

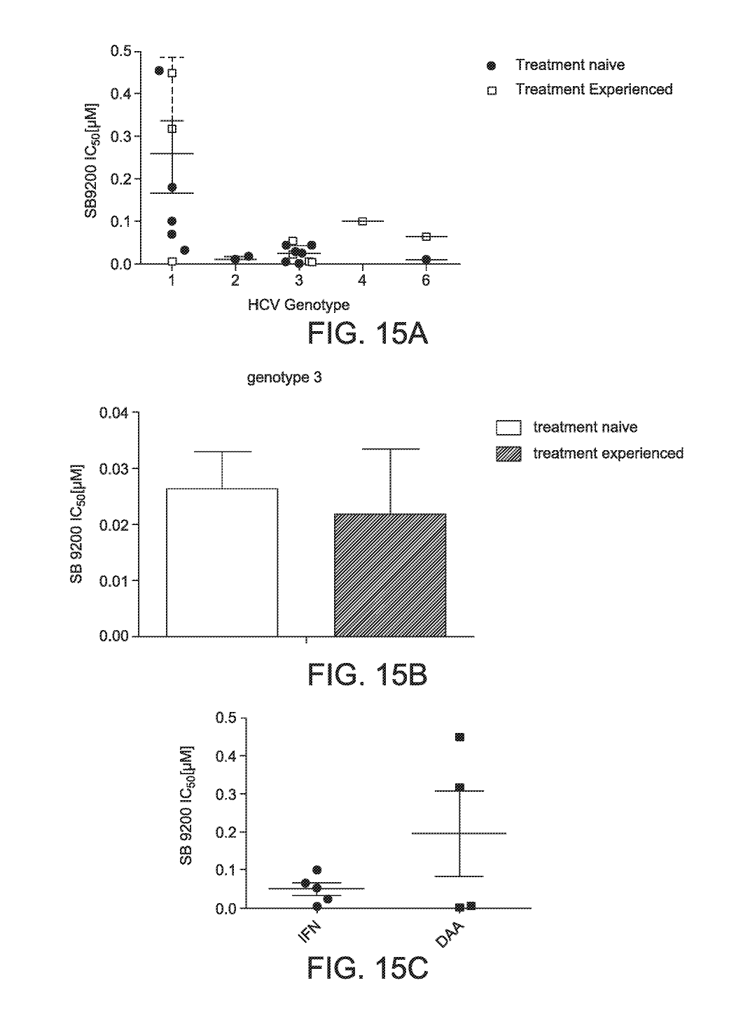

Results of these experiments as depicted in FIGS. 17-20 are shown with mean±sem and p values calculated using the Mann Whitney U test. As shown, replication of HCV from sera was inhibited by Formula (IIa) in a dose-dependent manner, with the IC₅₀ for all patient samples ranging from 0.001 μM to 0.455 μM. No significant difference in the HCV sensitivity to Formula (IIa) was found between the treatment naïve and treatment experienced group (for genotype 3, naïve IC₅₀ 0.026±0.007 μM, experienced IC₅₀ 0.022±0.12 μM). No difference in HCV sensitivity to Formula (IIa) was detected from a patient sample pre- and post-prg/RBV treatment (pre IC₅₀ 0.45 uM, post IC₅₀ 0.455 uM). The IC₅₀ for inhibition by Formula (IIa) between the peg/RBV and DAA group was not statistically significant. The results indicate that Formula (IIa) has a pan-genotypic activity, which is not affected by patient cirrhotic status nor influenced by previous treatment exposure. No difference was seen in the HCV sensitivity to Formula (IIa) in patients who had failed previous pegIFN/RBV or DAA treatment (FIGS. 15A-15C).

### Example 4. Analysis of NS5A Mutations and IFN/Formula (IIa) IC₅₀ Values in Serum Samples from Patients Infected with Resistant Variants HCV

Serum samples were taken from 8 patients infected with drug-resistant strains of HCV (FIG. 17). HCV RNA was isolated from the samples and mutations in the NS5A gene were determined through qPCR. The IC₅₀ values of both Formula (IIa) and IFN were determined through the capture fusion assay. Briefly, pre-stimulated THP-1 cells were infected with serum from donors chronically infected with the common HCV genotypes G1 or G3, or from patients infected with the less prevalent G2, G4 or G6 HCV. The hybrid cells were treated once with a range of concentrations of Formula (IIa). For comparison, fused cells infected with G1 and G3 sera were treated with telaprevir or alisporivir. The cells were cultured for 5 days, before quantification of HCV RNA by PCR. Dose-response curves were used to calculate IC₅₀ values for each experiment. The detailed assay protocol is outlined below.

### Cells, Reagents and Clinical Material

Huh7.5 and Huh7 cells were maintained in Dulbecco's modified Eagle medium with glutamine supplemented with 10% fetal calf serum and 1% penicillin/streptomycin (DMEM/10% FCS/PS). THP-1 cells were maintained in RPMI with glutamine supplemented with 10% fetal calf serum and 1% penicillin/streptomycin (RPMI/10% FCS/PS). Peripheral blood mononuclear cells (PBMC) and sera were obtained from patients with chronic HCV infection, with informed consent. Cytokines used were phorbol 12-myristate 13-acetate (PMA, Sigma-Aldrich, Dorset, UK), IFNγ (Invitrogen, Paisley, UK) and osteopontin (R&D Systems, Abingdon, UK). Primary antibodies used were anti-albumin (Dako, Glostrup, Denmark), anti-CD81 (BD Biosciences, Oxford, UK), anti-SR-B1 (Novus Biologicals, Cambridge, UK), anti-CD32 and anti-CD64 (Abcam, Cambridge, UK). Mouse IgG1 isotype control was purchased from Biolegend UK (London, UK). Fluorescent secondary antibodies AlexaFluor488 and 594 anti-sheep, anti-mouse and anti-rabbit IgG were purchased from Invitrogen.

### Fusion of Patient Monocytes

PBMCs were separated from whole blood by centrifugation on Ficoll-Paque. The PBMC layer was washed twice before positive selection of CD14+ cells by magnetic separation. Huh7.5 cells were trypsinized and combined with CD14+ cells at a 1:1 ratio. After removal of all supernatant, the cell pellet was slowly resuspended in prewarmed polyethylene glycol 1500 (Roche Diagnostics, Burgess Hill, UK). After incubation at 37° C. for 2 minutes, prewarmed medium (DMEM/10% FCS/PS) was added dropwise and the cells washed by centrifugation. The fused cells were seeded into 6 well plates at a density of 5×10⁵ cells/mL and maintained at 37° C. until RNA extraction.

### Stimulation and Infection of THP-1 Cells

THP-1 cells were seeded into 6 well plates at a density of 10⁶ cells/mL and maintained in RPMI supplemented with 10% fetal calf serum and 1% penicillin/streptomycin (RPMI/10% FCS/1% PS) for 18 hours, with or without the addition of cytokines. The cells were washed three times and the medium replaced with RPMI/2% FCS and patient serum at a ratio of 1 HCV copy per cell. For antibody blocking experiments, cells were incubated with blocking antibody at 37° C. for one hour before addition of patient serum. After incubation at 37° C. for 18-24 hours, the supernatant was removed and cells were washed three times. Adherent cells were removed using a cell scraper and Huh7.5 cells added at a 1:1 ratio. Cell fusion was performed as described above. The fused cells were seeded into 6 well plates at a density of 10⁵ cells/mL and maintained at 37° C. in the presence or absence of antiviral drugs for up to 7 days. In selected experiments, supernatants were pooled from non-drug treated wells and concentrated on a sucrose gradient. In brief, the pooled supernatants were filtered through a 0.45 μm filter then 10 mL of each was layered on 4 mL 20% sucrose. The supernatants were centrifuged at 24,000×g for 2 hours and the resulting pellet resuspended in 1 mL RPMI. For capture-fusion experiments using concentrated supernatant, 10⁶ prestimulated THP-1 cells were incubated with 1 mL of each concentrated supernatant for 24 hours before fusion, as described above.

### Drug Inhibition Assays

Fused cells were rested overnight prior to addition of antiviral drugs. Telaprevir and other DAAs, as well as alisporivir were stored as 20 mM stock solutions in dimethyl sulfoxide (DMSO). Fresh dilutions were made for each experiment and each drug concentration was tested in quadruplicate. Drug dilution mix alone was added to the control wells (RPMI/2% FCS/0.05% DMSO). Medium and drug were refreshed at day 3 after fusion. After quantification of HCV RNA, viral RNA was calculated as a percentage of that present in the untreated wells. Dose-response curves were constructed and used to estimate the 50% inhibitory concentration (IC₅₀) of drug for each sample using Prism 4.0 software (GraphPad, La Jolla, Calif.).

### Results

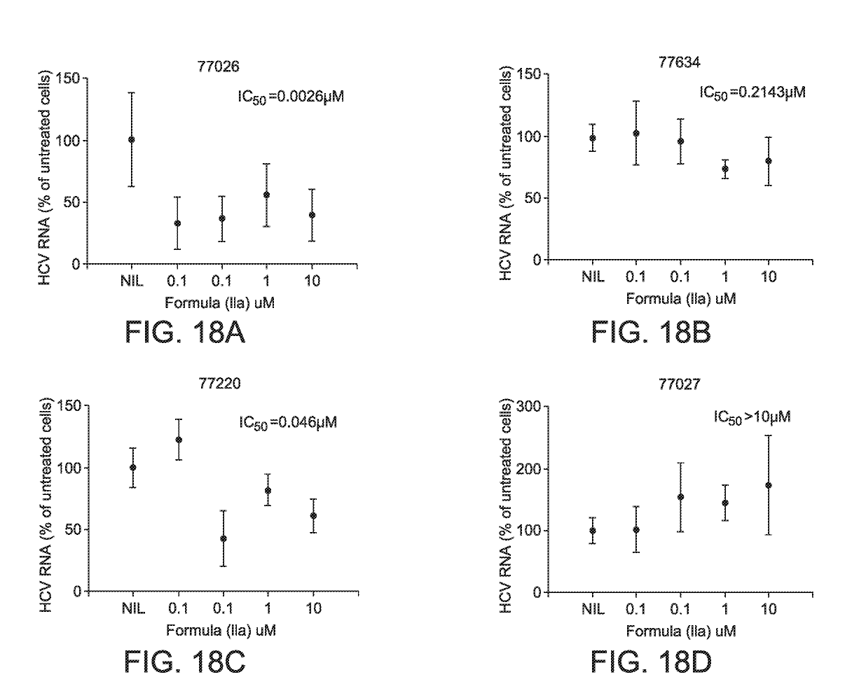

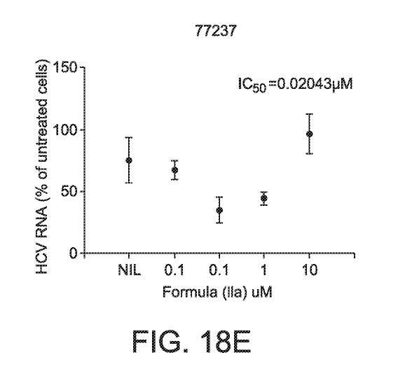

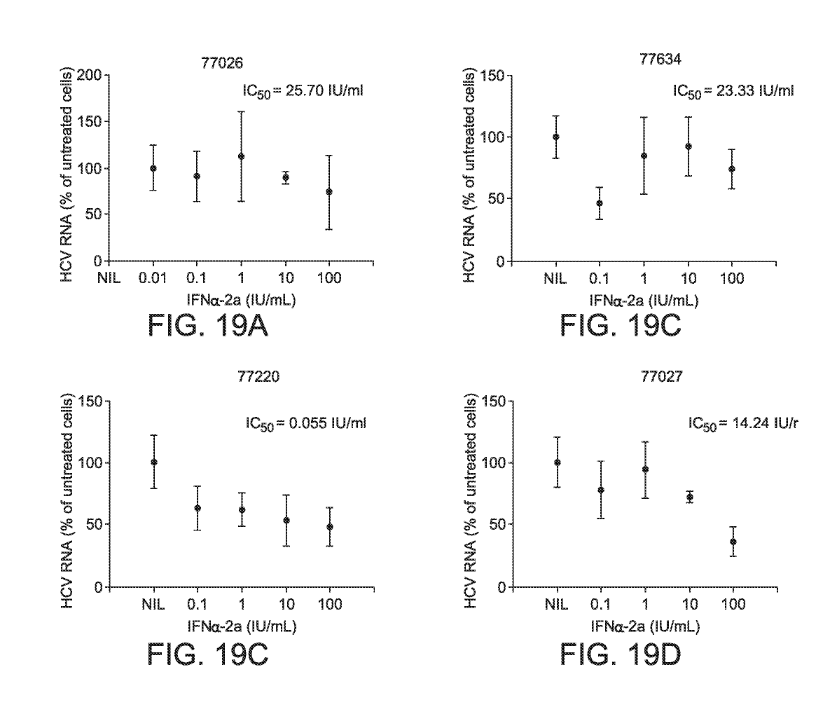

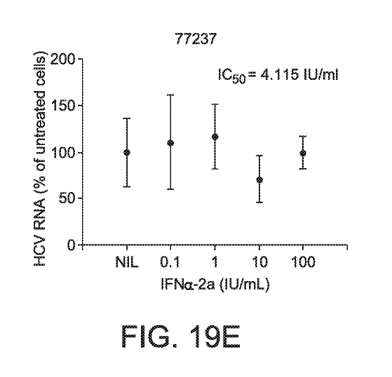

Results of these experiments as depicted in FIGS. 17-19 are shown with mean±sem and p values calculated using the Mann Whitney U test. As shown, replication of HCV from sera was inhibited by Formula (IIa) in a dose-dependent manner, in which the Formula (IIa) IC₅₀ value from five selected patient samples ranged from 0.001 μM to >10 μM (FIGS. 18A-18E). The corresponding sensitivity to IFN in these samples is shown in FIGS. 19A-19F.

### Example 5. Sequence Analysis of Samples from HCV-Infected Patients Bearing a Variant Form of the NS5A Protein

Serum samples were taken from 2 patients infected with drug-resistant strains of HCV. The first sample was taken from a patient prior to treatment with Formula (IL) (FIG. 20), while the second sample was obtained from a patient after failure of EAP treatment (FIG. 21). The HCV RNA was isolated from each sample and the NS5A locus of each sample was subjected to sequence analysis to determine the pattern of amino acid substitution at amino acid positions L31 and Y93 in the resulting NS5A protein.

### Example 6. Additional Studies of Formula (IIa) Activity in Patients Infected with Resistant Strains of HCV

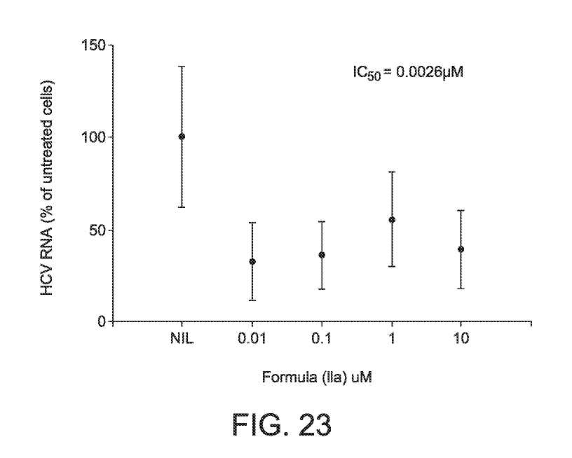

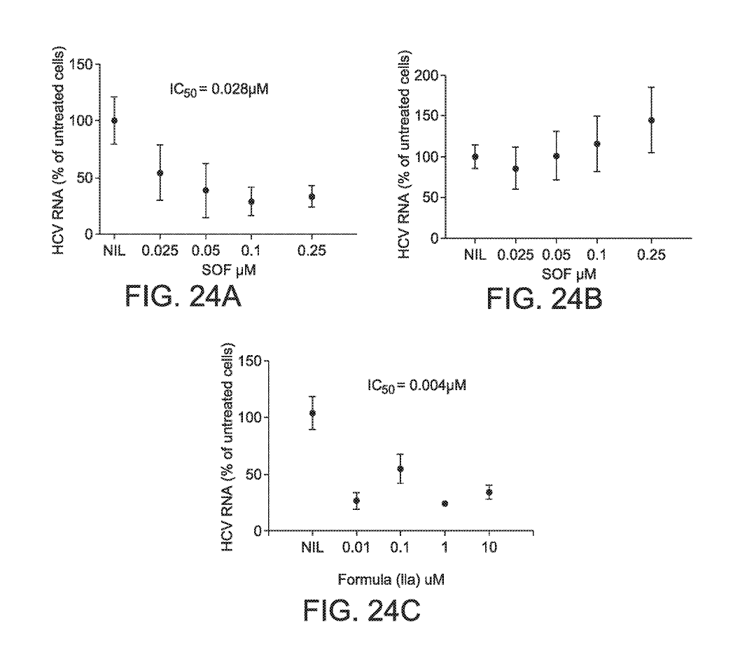

Serum samples were taken from 12 patients infected with drug-resistant strains of HCV that have previously not responded to current anti-HCV treatment regimes, as summarized in FIG. 22. Viruses with known variants that are associated with poor response to current NS5A inhibitors were included in the assay. As described in Example 1, THP-1 cells were exposed to donor serum, fused with Huh7 derivative cells and treated with differing concentrations of Formula (IIa) before qPCR assessment of HCV replication. FIG. 22 charts the Formula (IIa) responses from a panel of treatment failures with various NS5A mutations. Representative data from a patient with the NS5A resistance motif L31M is shown in FIG. 23. FIG. 24 summarizes the response to sofosbuvir in pre-treatment (FIG. 24A) and post-treatment (FIG. 24B) samples from a patient who relapsed after receiving a sofosbuvir-containing therapy and had exhibited no known sofosbuvir resistance motifs. The post treatment sample was sensitive to Formula (IIa) (FIG. 24C).

### EQUIVALENTS

The disclosures of each and every patent, patent application, and publication cited herein are hereby incorporated herein by reference in their entirety. While this disclosure has been described with reference to specific aspects, it is apparent that other aspects and variations may be devised by others skilled in the art without departing from the true spirit and scope of the disclosure. The appended claims are intended to be construed to include all such aspects and equivalent variations. Any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference.

While this disclosure has been particularly shown and described with references to preferred embodiments thereof, it will be understood by those skilled in the art that various changes in form and details may be made therein without departing from the scope of the disclosure encompassed by the appended claims.

1. A method of treating a subject infected with the Hepatitis C virus, the method comprising administering to the subject a pharmaceutical composition comprising a compound of Formula (I) at a dosage of about 10 mg to about 1500 mg, wherein the compound is selected from: 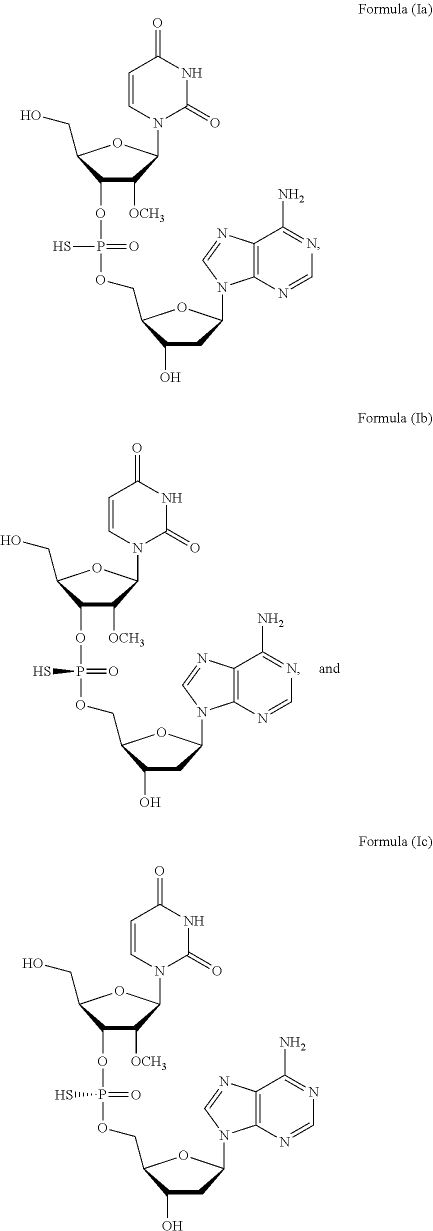
or a prodrug or pharmaceutically acceptable salt thereof to thereby treat the subject.

2. The method of claim 1, wherein the prodrug of Formula (I) is a compound of Formula (II), wherein the compound is selected from: 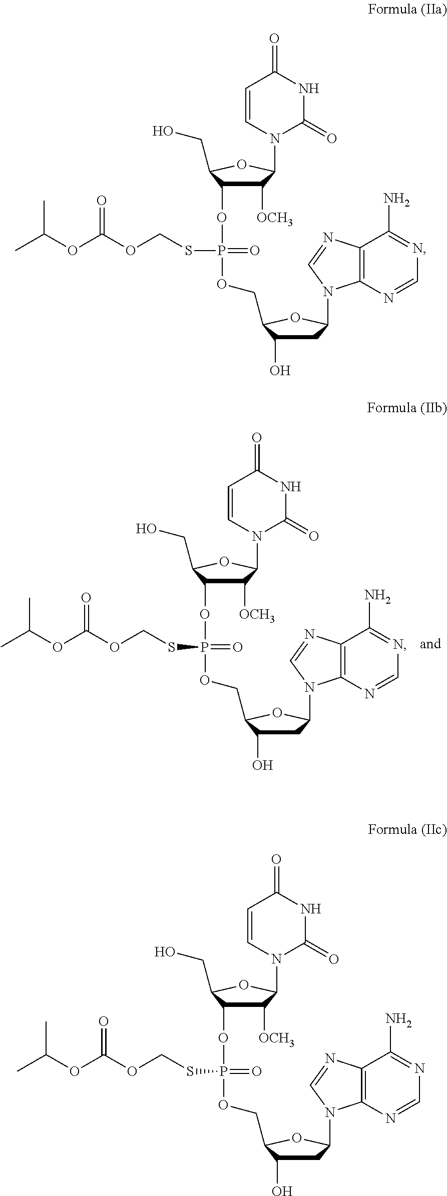
or a pharmaceutically acceptable salt thereof.

3. The method of claim 1, wherein the composition comprises a mixture of compounds of Formula (I), e.g., Formula (Ib) and Formula (Ic).

4-5. (canceled)

6. The method of claim 2, wherein the composition comprises a mixture of compounds of Formula (II), e.g., Formula (IIb) and Formula (IIc).

7-8. (canceled)

9. The method of claim 1, wherein the compound of Formula (I) is administered orally (e.g., the compound of Formula (I) is administered orally).

10-26. (canceled)

27. A method for treating a subject infected with the Hepatitis C virus, the method comprising administering to the subject a compound of Formula (I), wherein the compound is selected from: 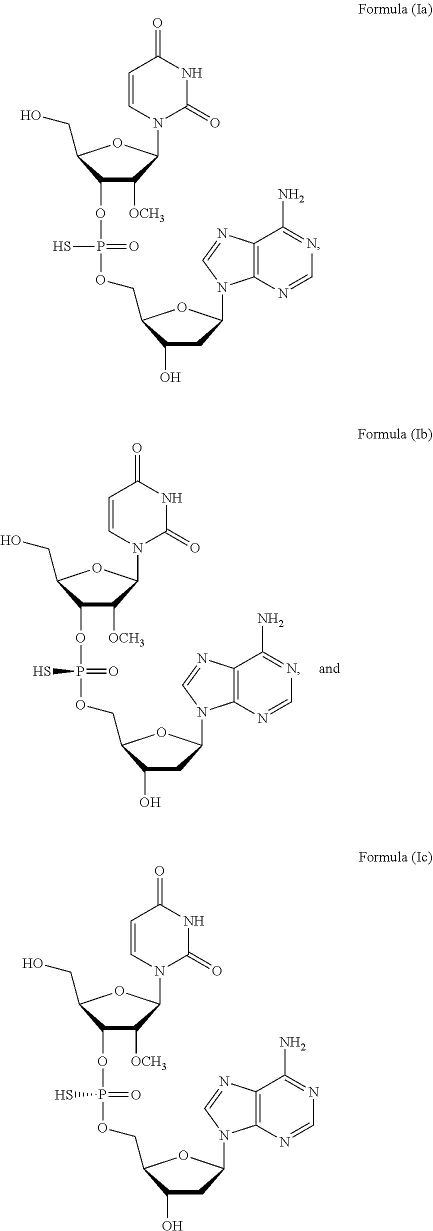
or a prodrug or pharmaceutically acceptable salt thereof in combination with sofosbuvir to thereby treat the subject.

28. The method of claim 27, wherein the prodrug of Formula (I) is a compound of Formula (II), wherein the compound is selected from: 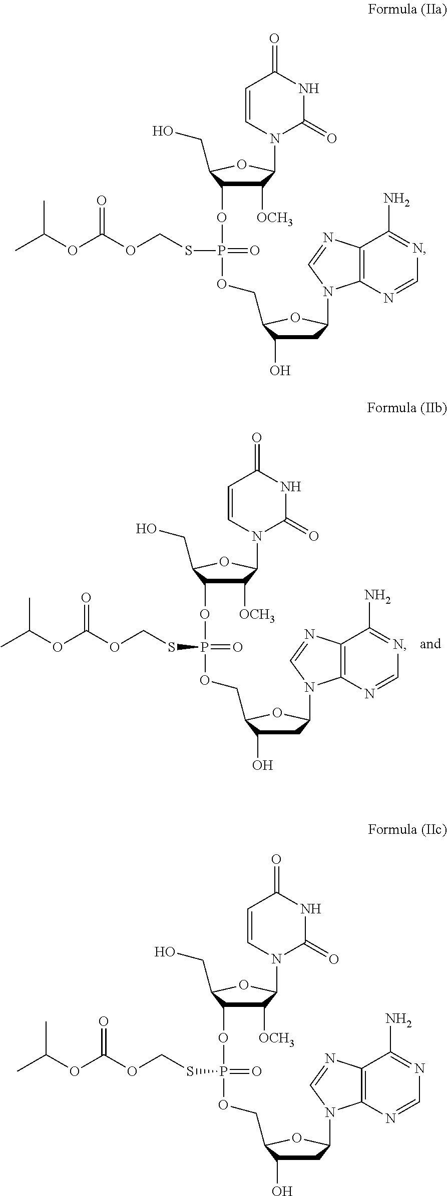
or a pharmaceutically acceptable salt thereof.

29. The method of claim 27, wherein the method comprises administering to the subject a compound of Formula (I), e.g., Formula (Ia), Formula (Ib), or Formula (Ic) or a pharmaceutically acceptable salt thereof, in combination with sofosbuvir.

30. (canceled)

31. The method of claim 28, wherein the method comprises administering to the subject a compound of Formula (II), e.g., Formula (IIa), Formula (IIb), or Formula (IIc) or a pharmaceutically acceptable salt thereof, in combination with sofosbuvir.

32-38. (canceled)

39. The method of claim 27, wherein the combination of a compound of Formula (I) and sofosbuvir is administered orally.

40-41. (canceled)

42. A kit comprising sofosbuvir and a compound of Formula (I) or Formula (II) or a pharmaceutically acceptable salt thereof.

43. (canceled)

44. A method of treating a subject infected with a drug-resistant strain of the Hepatitis C virus (HCV), the method comprising administering to the subject a compound of Formula (I), wherein the compound is selected from: 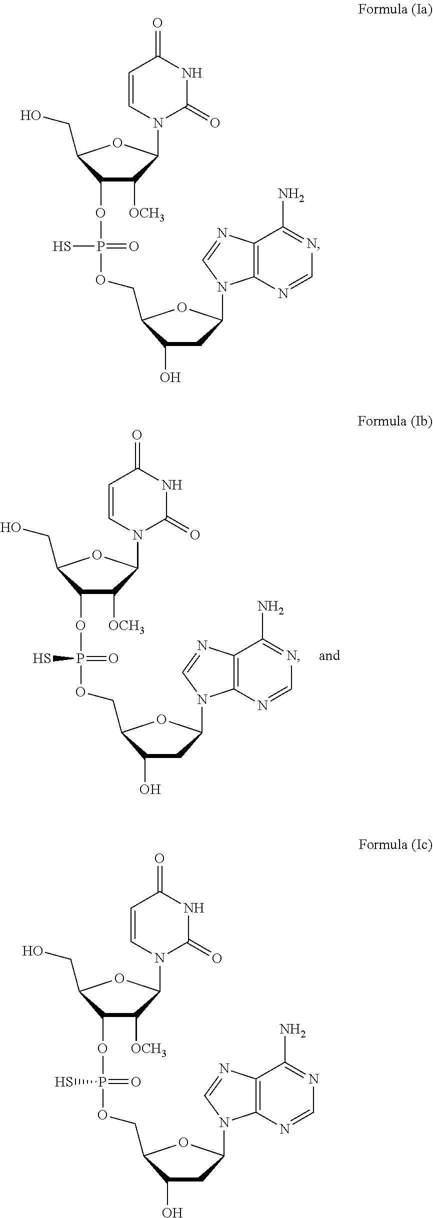
or a prodrug or pharmaceutically acceptable salt thereof to thereby treat the subject.

45. The method of claim 44, wherein the prodrug of Formula (I) is a compound of Formula (II), wherein the compound is selected from: 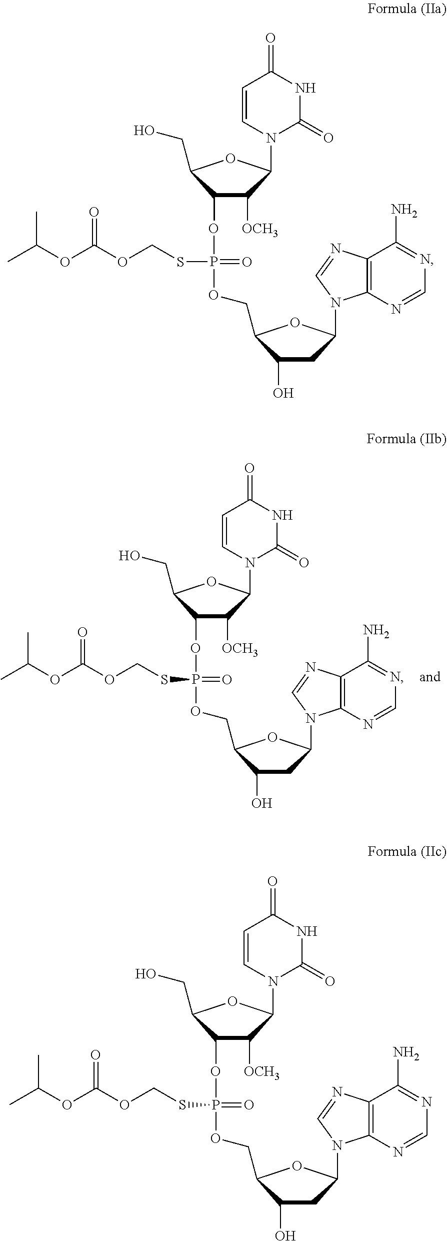
or a pharmaceutically acceptable salt thereof.

46. The method of claim 44, wherein the drug-resistant strain of HCV is resistant to an anti-HCV agent other than a compound of Formula (I) or Formula (II) or a pharmaceutically acceptable salt thereof.

47-63. (canceled)

64. A method of treating a subject infected with the Hepatitis C virus (HCV) that has previously been administered an anti-HCV agent, the method comprising administering to the subject a compound of Formula (I), wherein the compound is selected from: 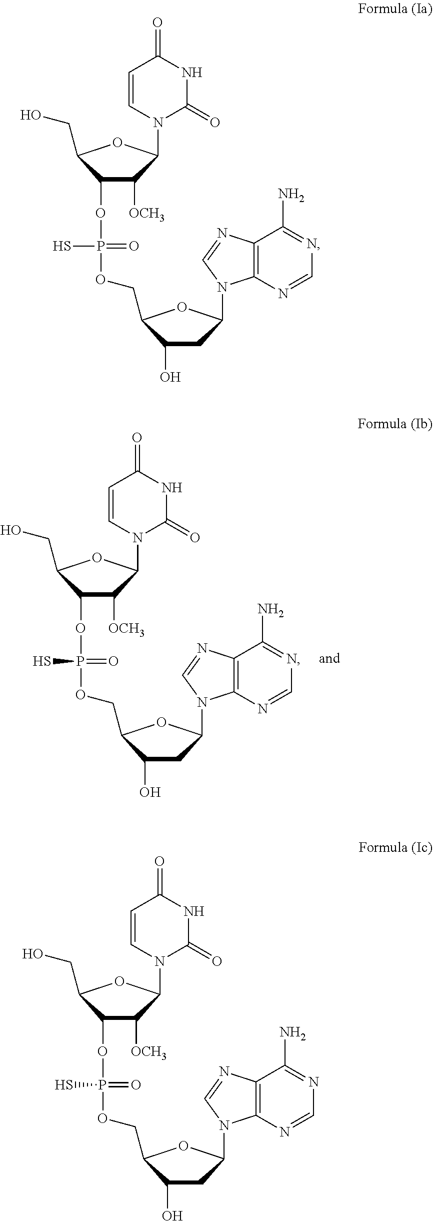
or a prodrug or pharmaceutically acceptable salt thereof to thereby treat the subject.

65. The method of claim 64, wherein the prodrug of Formula (I) is a compound of Formula (II), wherein the compound is selected from: 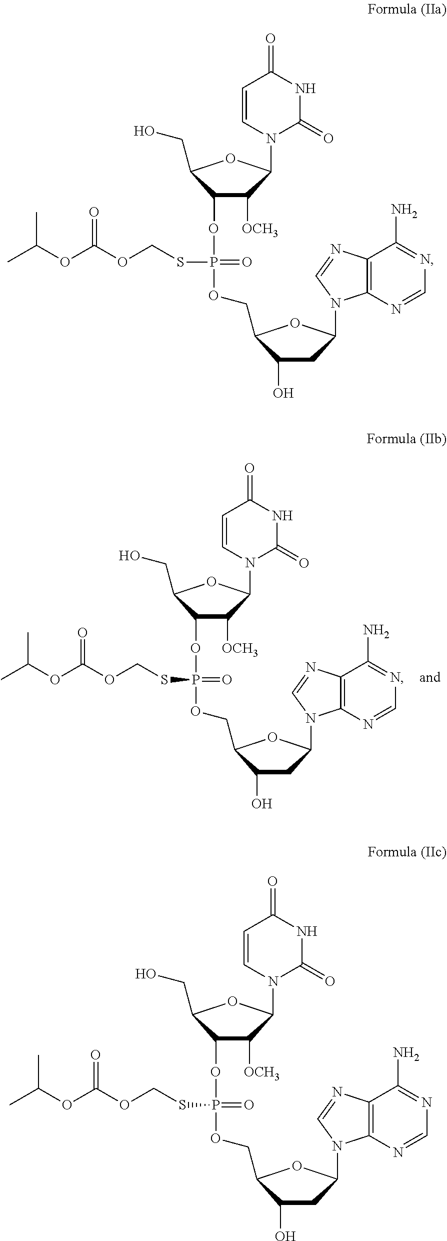
or a pharmaceutically acceptable salt thereof.

66. The method of claim 64, wherein the HCV strain is a drug-resistant HCV strain.

67. The method of claim 66, wherein the drug-resistant strain HCV strain is resistant to an anti-HCV agent other than a compound of Formula (I) or Formula (II) or a pharmaceutically acceptable salt thereof.

68-104. (canceled)

105. The method of claim 44, wherein the subject is further administered an additional agent or treatment, or pharmaceutically acceptable salt thereof.

106-107. (canceled)

108. The method of claim 44, wherein a compound of Formula (I) or Formula (II) is formulated as a pharmaceutical composition.

109. (canceled)
